SEQUENCE LISTING (162 sequences)

SEQ ID NO: 1 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
  source 1..1307: note = Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1300 aa, Francisella tularensis
  source 1..1300: note = Francisella tularensis subsp. novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 5 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 6 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 7 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 8 - protein, 1306 aa, Thiomicrospira sp.
  source 1..1306: note = Thiomicrospira sp. XS5
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 9 - protein, 1214 aa, Btyrivibrio sp.
  source 1..1214: note = Btyrivibrio sp. NC3005
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 10 - protein, 1129 aa, unidentified
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 11 - RNA, 20 nt, unidentified
  source 1..20: mol_type = genomic RNA; note = Lachnospiraceae bacterium
aatttctactaagtgtagat

SEQ ID NO: 12 - RNA, 19 nt, Acidaminococcus sp.
  source 1..19: mol_type = genomic RNA; strain = Acidaminococcus sp. BV3L6
aatttctactcttgtagat

SEQ ID NO: 13 - RNA, 19 nt, Francisella tularensis
  source 1..19: mol_type = genomic RNA; sub_species = novicida
aatttctactgttgtagat

SEQ ID NO: 14 - RNA, 19 nt, Porphyromonas macacae
  source 1..19: mol_type = genomic RNA
aatttctactattgtagat

SEQ ID NO: 15 - RNA, 20 nt, Moraxella bovoculi
  source 1..20: mol_type = genomic RNA
aatttctactgtttgtagat

SEQ ID NO: 16 - RNA, 19 nt, Thiomicrospira sp.
  source 1..19: mol_type = genomic RNA; strain = Thiomicrospira sp. XS5
aatttctactgttgtagat

SEQ ID NO: 17 - RNA, 19 nt, Btyrivibrio sp.
  source 1..19: mol_type = genomic RNA; strain = Btyrivibrio sp. NC3005
aatttctactattgtagat

SEQ ID NO: 18 - RNA, 10 nt, synthetic construct
  source 1..10: mol_type = other RNA
aaaaaaaaaa

SEQ ID NO: 19 - RNA, 91 nt, unidentified
  source 1..91: mol_type = genomic RNA
gtctagaggacagaatttttcaacgggtgtgccaatggccactttccaggtggcaaagcccgttgagcttctcaaatctgagaagtggcac

SEQ ID NO: 20 - RNA, 41 nt, synthetic construct
  source 1..41: mol_type = other RNA
taatttctactaagtgtagatcgtcgccgtccagctcgacc

SEQ ID NO: 21 - RNA, 45 nt, synthetic construct
  source 1..45: mol_type = other RNA
taatttctactaagtgtagatcaacgtcgtgactgggaaaaccct

SEQ ID NO: 22 - RNA, 41 nt, synthetic construct
  source 1..41: mol_type = other RNA
taatttctactaagtgtagataacgaaccaccagcagaaga

SEQ ID NO: 23 - RNA, 41 nt, synthetic construct
  source 1..41: mol_type = other RNA
taatttctactaagtgtagatgatcgttacgctaactatga

SEQ ID NO: 24 - RNA, 41 nt, synthetic construct
  source 1..41: mol_type = other RNA
taatttctactaagtgtagatcctgggtgttccacagctga

SEQ ID NO: 25 - RNA, 41 nt, synthetic construct
  source 1..41: mol_type = other RNA
taatttctactaagtgtagatctacattacaggctaacaaa

SEQ ID NO: 26 - RNA, 41 nt, synthetic construct
  source 1..41: mol_type = other RNA
taatttctactaagtgtagatgtacattgcaagatactaaa

SEQ ID NO: 27 - RNA, 41 nt, synthetic construct
  source 1..41: mol_type = other RNA
taatttctactaagtgtagattgaagtagatatggcagcac

SEQ ID NO: 28 - RNA, 41 nt, synthetic construct
  source 1..41: mol_type = other RNA
taatttctactaagtgtagatacaatatgtgcttctacaca

SEQ ID NO: 29 - RNA, 40 nt, synthetic construct
  source 1..40: mol_type = other RNA
taatttctactcttgtagatgatcgttacgctaactatga

SEQ ID NO: 30 - RNA, 40 nt, synthetic construct
  source 1..40: mol_type = other RNA
taatttctactgttgtagatgatcgttacgctaactatga

SEQ ID NO: 31 - RNA, 111 nt, synthetic construct
  source 1..111: mol_type = other RNA
gtctagaggacagaatttttcaacgggtgtgccaatggccactttccaggtggcaaagcccgttgagcttctcaaatctgagaagtggcacgatcgttacgctaactatga

SEQ ID NO: 32 - RNA, 116 nt, synthetic construct
  source 1..116: mol_type = other RNA
cgtcgccgtccagctcgaccgttttagagctatgctgttttggaaacaaaacagcatagcaagttaaaataaggctagtccgttatcaacttgaaaaagtggcaccgagtcggtgc

SEQ ID NO: 33 - RNA, 116 nt, synthetic construct
  source 1..116: mol_type = other RNA
aacgaaccaccagcagaagagttttagagctatgctgttttggaaacaaaacagcatagcaagttaaaataaggctagtccgttatcaacttgaaaaagtggcaccgagtcggtgc

SEQ ID NO: 34 - RNA, 116 nt, synthetic construct
  source 1..116: mol_type = other RNA
gatcgttacgctaactatgagttttagagctatgctgttttggaaacaaaacagcatagcaagttaaaataaggctagtccgttatcaacttgaaaaagtggcaccgagtcggtgc

SEQ ID NO: 35 - RNA, 135 nt, synthetic construct
  source 1..135: mol_type = other RNA
gatcgttacgctaactatgagttgtagctccctttctcatttcgcagtgcgaaagcactgcgaaatgagaaccgttgctacaataaggccgtctgaaaagatgtgccgcaacgctctgccccttaaagcttctgc

SEQ ID NO: 36 - RNA, 139 nt, synthetic construct
  source 1..139: mol_type = other RNA
gatcgttacgctaactatgagttttagtccctttttaaatttctttatggtaaaattataatctcataagaaatttaaaaagggactaaaataaagagtttgcgggactctgcggggttacaatcccctaaaaccgctt

SEQ ID NO: 37 - RNA, 55 nt, synthetic construct
  source 1..55: mol_type = other RNA
gccggggtggtgcccatcctggtcgagctggacggcgacgtaaacggccacaagc

SEQ ID NO: 38 - RNA, 55 nt, synthetic construct
  source 1..55: mol_type = other RNA
tagcattccacagacagccctcatagttagcgtaacgatctaaagttttgtcgtc

SEQ ID NO: 39 - DNA, 58 bp, synthetic construct
  source 1..58: mol_type = other DNA
agcttgtctgccatggacatgcagactatactgttattgttgtacagaccgaattccc

SEQ ID NO: 40 - DNA, 58 bp, synthetic construct
  source 1..58: mol_type = other DNA
gggaattcggtctgtacaacaataacagtatagtctgcatgtccatggcagacaagct

SEQ ID NO: 41 - DNA, 55 bp, synthetic construct
  source 1..55: mol_type = other DNA
gcttgtggccgtttacgtcgccgtccagctcgaccaggatgggcaccaccccggc

SEQ ID NO: 42 - DNA, 55 bp, synthetic construct
  source 1..55: mol_type = other DNA
gccggggtggtgcccatcctggtcgagctggacggcgacgtaaacggccacaagc

SEQ ID NO: 43 - DNA, 55 bp, synthetic construct
  source 1..55: mol_type = other DNA
gcttgtggccgtttacgtcgccgtccagctcgaggaggatgggcaccaccccggc

SEQ ID NO: 44 - DNA, 55 bp, synthetic construct
  source 1..55: mol_type = other DNA
gccggggtggtgcccatcctcctcgagctggacggcgacgtaaacggccacaagc

SEQ ID NO: 45 - DNA, 55 bp, synthetic construct
  source 1..55: mol_type = other DNA
gcttgtggccgtttacgtcgccgtccagctcctccaggatgggcaccaccccggc

SEQ ID NO: 46 - DNA, 55 bp, synthetic construct
  source 1..55: mol_type = other DNA
gccggggtggtgcccatcctggaggagctggacggcgacgtaaacggccacaagc

SEQ ID NO: 47 - DNA, 55 bp, synthetic construct
  source 1..55: mol_type = other DNA
gcttgtggccgtttacgtcgccgtccagcaggaccaggatgggcaccaccccggc

SEQ ID NO: 48 - DNA, 55 bp, synthetic construct
  source 1..55: mol_type = other DNA
gccggggtggtgcccatcctggtcctgctggacggcgacgtaaacggccacaagc

SEQ ID NO: 49 - DNA, 55 bp, synthetic construct
  source 1..55: mol_type = other DNA
gcttgtggccgtttacgtcgccgtccacgtcgaccaggatgggcaccaccccggc

SEQ ID NO: 50 - DNA, 55 bp, synthetic construct
  source 1..55: mol_type = other DNA
gccggggtggtgcccatcctggtcgacgtggacggcgacgtaaacggccacaagc

SEQ ID NO: 51 - DNA, 55 bp, synthetic construct
  source 1..55: mol_type = other DNA
gcttgtggccgtttacgtcgccgtcgtgctcgaccaggatgggcaccaccccggc

SEQ ID NO: 52 - DNA, 55 bp, synthetic construct
  source 1..55: mol_type = other DNA
gccggggtggtgcccatcctggtcgagcacgacggcgacgtaaacggccacaagc

SEQ ID NO: 53 - DNA, 55 bp, synthetic construct
  source 1..55: mol_type = other DNA
gcttgtggccgtttacgtcgccgagcagctcgaccaggatgggcaccaccccggc

SEQ ID NO: 54 - DNA, 55 bp, synthetic construct
  source 1..55: mol_type = other DNA
gccggggtggtgcccatcctggtcgagctgctcggcgacgtaaacggccacaagc

SEQ ID NO: 55 - DNA, 55 bp, synthetic construct
  source 1..55: mol_type = other DNA
gcttgtggccgtttacgtcgcgctccagctcgaccaggatgggcaccaccccggc

SEQ ID NO: 56 - DNA, 55 bp, synthetic construct
  source 1..55: mol_type = other DNA
gccggggtggtgcccatcctggtcgagctggagcgcgacgtaaacggccacaagc

SEQ ID NO: 57 - DNA, 55 bp, synthetic construct
  source 1..55: mol_type = other DNA
gcttgtggccgtttacgtccgcgtccagctcgaccaggatgggcaccaccccggc

SEQ ID NO: 58 - DNA, 55 bp, synthetic construct
  source 1..55: mol_type = other DNA
gccggggtggtgcccatcctggtcgagctggacgcggacgtaaacggccacaagc

SEQ ID NO: 59 - DNA, 55 bp, synthetic construct
  source 1..55: mol_type = other DNA
gcttgtggccgtttacgaggccgtccagctcgaccaggatgggcaccaccccggc

SEQ ID NO: 60 - DNA, 55 bp, synthetic construct
  source 1..55: mol_type = other DNA
gccggggtggtgcccatcctggtcgagctggacggcctcgtaaacggccacaagc

SEQ ID NO: 61 - DNA, 55 bp, synthetic construct
  source 1..55: mol_type = other DNA
gcttgtggccgtttagctcgccgtccagctcgaccaggatgggcaccaccccggc

SEQ ID NO: 62 - DNA, 55 bp, synthetic construct
  source 1..55: mol_type = other DNA
gccggggtggtgcccatcctggtcgagctggacggcgagctaaacggccacaagc

SEQ ID NO: 63 - DNA, 55 bp, synthetic construct
  source 1..55: mol_type = other DNA
gcttgtggccgagcacgtcgccgtccagctcgaccaggatgggcaccaccccggc

SEQ ID NO: 64 - DNA, 55 bp, synthetic construct
  source 1..55: mol_type = other DNA
gccggggtggtgcccatcctggtcgagctggacggcgacgtgctcggccacaagc

SEQ ID NO: 65 - DNA, 20 bp, synthetic construct
  source 1..20: mol_type = other DNA
cgacgtaaacggccacaagc

SEQ ID NO: 66 - DNA, 25 bp, synthetic construct
  source 1..25: mol_type = other DNA
gacggcgacgtaaacggccacaagc

SEQ ID NO: 67 - DNA, 30 bp, synthetic construct
  source 1..30: mol_type = other DNA
agctggacggcgacgtaaacggccacaagc

SEQ ID NO: 68 - DNA, 35 bp, synthetic construct
  source 1..35: mol_type = other DNA
ggtcgagctggacggcgacgtaaacggccacaagc

SEQ ID NO: 69 - DNA, 40 bp, synthetic construct
  source 1..40: mol_type = other DNA
atcctggtcgagctggacggcgacgtaaacggccacaagc

SEQ ID NO: 70 - DNA, 20 bp, synthetic construct
  source 1..20: mol_type = other DNA
gcttgtggccgtttacgtcg

SEQ ID NO: 71 - DNA, 25 bp, synthetic construct
  source 1..25: mol_type = other DNA
gcttgtggccgtttacgtcgccgtc

SEQ ID NO: 72 - DNA, 30 bp, synthetic construct
  source 1..30: mol_type = other DNA
gcttgtggccgtttacgtcgccgtccagct

SEQ ID NO: 73 - DNA, 35 bp, synthetic construct
  source 1..35: mol_type = other DNA
gcttgtggccgtttacgtcgccgtccagctcgacc

SEQ ID NO: 74 - DNA, 40 bp, synthetic construct
  source 1..40: mol_type = other DNA
gcttgtggccgtttacgtcgccgtccagctcgaccaggat

SEQ ID NO: 75 - DNA, 55 bp, synthetic construct
  source 1..55: mol_type = other DNA
gacgacaaaactttagatcgttacgctaactatgagggctgtctgtggaatgcta

SEQ ID NO: 76 - DNA, 55 bp, synthetic construct
  source 1..55: mol_type = other DNA
tagcattccacagacagccctcatagttagcgtaacgatctaaagttttgtcgtc

SEQ ID NO: 77 - DNA, 55 bp, synthetic construct
  source 1..55: mol_type = other DNA
gacgacaaaactttagatcgttacgctaactatctgggctgtctgtggaatgcta

SEQ ID NO: 78 - DNA, 55 bp, synthetic construct
  source 1..55: mol_type = other DNA
tagcattccacagacagcccagatagttagcgtaacgatctaaagttttgtcgtc

SEQ ID NO: 79 - DNA, 55 bp, synthetic construct
  source 1..55: mol_type = other DNA
gacgacaaaactttagatcgttacgctaacttagagggctgtctgtggaatgcta

SEQ ID NO: 80 - DNA, 55 bp, synthetic construct
  source 1..55: mol_type = other DNA
tagcattccacagacagccctctaagttagcgtaacgatctaaagttttgtcgtc

SEQ ID NO: 81 - DNA, 55 bp, synthetic construct
  source 1..55: mol_type = other DNA
gacgacaaaactttagatcgttacgctaagaatgagggctgtctgtggaatgcta

SEQ ID NO: 82 - DNA, 55 bp, synthetic construct
  source 1..55: mol_type = other DNA
tagcattccacagacagccctcattcttagcgtaacgatctaaagttttgtcgtc

SEQ ID NO: 83 - DNA, 55 bp, synthetic construct
  source 1..55: mol_type = other DNA
gacgacaaaactttagatcgttacgctttctatgagggctgtctgtggaatgcta

SEQ ID NO: 84 - DNA, 55 bp, synthetic construct
  source 1..55: mol_type = other DNA
tagcattccacagacagccctcatagaaagcgtaacgatctaaagttttgtcgtc

SEQ ID NO: 85 - DNA, 55 bp, synthetic construct
  source 1..55: mol_type = other DNA
gacgacaaaactttagatcgttacggaaactatgagggctgtctgtggaatgcta

SEQ ID NO: 86 - DNA, 55 bp, synthetic construct
  source 1..55: mol_type = other DNA
tagcattccacagacagccctcatagtttccgtaacgatctaaagttttgtcgtc

SEQ ID NO: 87 - DNA, 55 bp, synthetic construct
  source 1..55: mol_type = other DNA
gacgacaaaactttagatcgttagcctaactatgagggctgtctgtggaatgcta

SEQ ID NO: 88 - DNA, 55 bp, synthetic construct
  source 1..55: mol_type = other DNA
tagcattccacagacagccctcatagttaggctaacgatctaaagttttgtcgtc

SEQ ID NO: 89 - DNA, 55 bp, synthetic construct
  source 1..55: mol_type = other DNA
gacgacaaaactttagatcgtatcgctaactatgagggctgtctgtggaatgcta

SEQ ID NO: 90 - DNA, 55 bp, synthetic construct
  source 1..55: mol_type = other DNA
tagcattccacagacagccctcatagttagcgatacgatctaaagttttgtcgtc

SEQ ID NO: 91 - DNA, 55 bp, synthetic construct
  source 1..55: mol_type = other DNA
gacgacaaaactttagatccatacgctaactatgagggctgtctgtggaatgcta

SEQ ID NO: 92 - DNA, 55 bp, synthetic construct
  source 1..55: mol_type = other DNA
tagcattccacagacagccctcatagttagcgtatggatctaaagttttgtcgtc

SEQ ID NO: 93 - DNA, 55 bp, synthetic construct
  source 1..55: mol_type = other DNA
gacgacaaaactttagaaggttacgctaactatgagggctgtctgtggaatgcta

SEQ ID NO: 94 - DNA, 55 bp, synthetic construct
  source 1..55: mol_type = other DNA
tagcattccacagacagccctcatagttagcgtaaccttctaaagttttgtcgtc

SEQ ID NO: 95 - DNA, 55 bp, synthetic construct
  source 1..55: mol_type = other DNA
gacgacaaaactttacttcgttacgctaactatgagggctgtctgtggaatgcta

SEQ ID NO: 96 - DNA, 55 bp, synthetic construct
  source 1..55: mol_type = other DNA
tagcattccacagacagccctcatagttagcgtaacgaagtaaagttttgtcgtc

SEQ ID NO: 97 - DNA, 55 bp, synthetic construct
  source 1..55: mol_type = other DNA
gacgacaaaacagcagatcgttacgctaactatgagggctgtctgtggaatgcta

SEQ ID NO: 98 - DNA, 55 bp, synthetic construct
  source 1..55: mol_type = other DNA
tagcattccacagacagccctcatagttagcgtaacgatctgctgttttgtcgtc

SEQ ID NO: 99 - DNA, 55 bp, synthetic construct
  source 1..55: mol_type = other DNA
gacgacaaaactttagatcgttacgctaactatgagggcgagttgtggaatgcta

SEQ ID NO: 100 - DNA, 55 bp, synthetic construct
  source 1..55: mol_type = other DNA
tagcattccacaactcgccctcatagttagcgtaacgatctaaagttttgtcgtc

SEQ ID NO: 101 - DNA, 55 bp, synthetic construct
  source 1..55: mol_type = other DNA
gacgacaaaactttagatcgttacgctaactatgagggccaaatgtggaatgcta

SEQ ID NO: 102 - DNA, 55 bp, synthetic construct
  source 1..55: mol_type = other DNA
tagcattccacatttggccctcatagttagcgtaacgatctaaagttttgtcgtc

SEQ ID NO: 103 - DNA, 55 bp, synthetic construct
  source 1..55: mol_type = other DNA
agttgtgttagtttacctgggtgttccacagctgatagtgattgccttgaataaa

SEQ ID NO: 104 - DNA, 55 bp, synthetic construct
  source 1..55: mol_type = other DNA
tttattcaaggcaatcactatcagctgtggaacacccaggtaaactaacacaact

SEQ ID NO: 105 - DNA, 55 bp, synthetic construct
  source 1..55: mol_type = other DNA
agttgtgttagtttacctgggtgttccacagctcttagtgattgccttgaataaa

SEQ ID NO: 106 - DNA, 55 bp, synthetic construct
  source 1..55: mol_type = other DNA
tttattcaaggcaatcactaagagctgtggaacacccaggtaaactaacacaact

SEQ ID NO: 107 - DNA, 55 bp, synthetic construct
  source 1..55: mol_type = other DNA
agttgtgttagtttacctgggtgttccacaggagatagtgattgccttgaataaa

SEQ ID NO: 108 - DNA, 55 bp, synthetic construct
  source 1..55: mol_type = other DNA
tttattcaaggcaatcactatctcctgtggaacacccaggtaaactaacacaact

SEQ ID NO: 109 - DNA, 55 bp, synthetic construct
  source 1..55: mol_type = other DNA
agttgtgttagtttacctgggtgttccactcctgatagtgattgccttgaataaa

SEQ ID NO: 110 - DNA, 55 bp, synthetic construct
  source 1..55: mol_type = other DNA
tttattcaaggcaatcactatcaggagtggaacacccaggtaaactaacacaact

SEQ ID NO: 111 - DNA, 55 bp, synthetic construct
  source 1..55: mol_type = other DNA
agttgtgttagtttacctgggtgttcctgagctgatagtgattgccttgaataaa

SEQ ID NO: 112 - DNA, 55 bp, synthetic construct
  source 1..55: mol_type = other DNA
tttattcaaggcaatcactatcagctcaggaacacccaggtaaactaacacaact

SEQ ID NO: 113 - DNA, 55 bp, synthetic construct
  source 1..55: mol_type = other DNA
agttgtgttagtttacctgggtgttggacagctgatagtgattgccttgaataaa

SEQ ID NO: 114 - DNA, 55 bp, synthetic construct
  source 1..55: mol_type = other DNA
tttattcaaggcaatcactatcagctgtccaacacccaggtaaactaacacaact

SEQ ID NO: 115 - DNA, 55 bp, synthetic construct
  source 1..55: mol_type = other DNA
agttgtgttagtttacctgggtgaaccacagctgatagtgattgccttgaataaa

SEQ ID NO: 116 - DNA, 55 bp, synthetic construct
  source 1..55: mol_type = other DNA
tttattcaaggcaatcactatcagctgtggttcacccaggtaaactaacacaact

SEQ ID NO: 117 - DNA, 55 bp, synthetic construct
  source 1..55: mol_type = other DNA
agttgtgttagtttacctgggacttccacagctgatagtgattgccttgaataaa

SEQ ID NO: 118 - DNA, 55 bp, synthetic construct
  source 1..55: mol_type = other DNA
tttattcaaggcaatcactatcagctgtggaagtcccaggtaaactaacacaact

SEQ ID NO: 119 - DNA, 55 bp, synthetic construct
  source 1..55: mol_type = other DNA
agttgtgttagtttacctgcctgttccacagctgatagtgattgccttgaataaa

SEQ ID NO: 120 - DNA, 55 bp, synthetic construct
  source 1..55: mol_type = other DNA
tttattcaaggcaatcactatcagctgtggaacaggcaggtaaactaacacaact

SEQ ID NO: 121 - DNA, 55 bp, synthetic construct
  source 1..55: mol_type = other DNA
agttgtgttagtttaccacggtgttccacagctgatagtgattgccttgaataaa

SEQ ID NO: 122 - DNA, 55 bp, synthetic construct
  source 1..55: mol_type = other DNA
tttattcaaggcaatcactatcagctgtggaacaccgtggtaaactaacacaact

SEQ ID NO: 123 - DNA, 55 bp, synthetic construct
  source 1..55: mol_type = other DNA
agttgtgttagtttaggtgggtgttccacagctgatagtgattgccttgaataaa

SEQ ID NO: 124 - DNA, 55 bp, synthetic construct
  source 1..55: mol_type = other DNA
tttattcaaggcaatcactatcagctgtggaacacccacctaaactaacacaact

SEQ ID NO: 125 - DNA, 55 bp, synthetic construct
  source 1..55: mol_type = other DNA
agttgtgttagagcacctgggtgttccacagctgatagtgattgccttgaataaa

SEQ ID NO: 126 - DNA, 55 bp, synthetic construct
  source 1..55: mol_type = other DNA
tttattcaaggcaatcactatcagctgtggaacacccaggtgctctaacacaact

SEQ ID NO: 127 - DNA, 23 bp, synthetic construct
  source 1..23: mol_type = other DNA
gcaaaccacctataggggaacac

SEQ ID NO: 128 - DNA, 21 bp, synthetic construct
  source 1..21: mol_type = other DNA
cagccaactcagcttcctttc

SEQ ID NO: 129 - DNA, 23 bp, synthetic construct
  source 1..23: mol_type = other DNA
catgccgccacgtctaatgtttc

SEQ ID NO: 130 - DNA, 21 bp, synthetic construct
  source 1..21: mol_type = other DNA
ggtgaagcacgcatacctgtg

SEQ ID NO: 131 - DNA, 31 bp, synthetic construct
  source 1..31: mol_type = other DNA
ttgttggggtaaccaactatttgttactgtt

SEQ ID NO: 132 - DNA, 29 bp, synthetic construct
  source 1..29: mol_type = other DNA
cctccccatgtctgaggtactccttaaag

SEQ ID NO: 133 - DNA, 37 bp, synthetic construct
  source 1..37: mol_type = other DNA
gcataatcaattatttgttactgtggtagataccact

SEQ ID NO: 134 - DNA, 34 bp, synthetic construct
  source 1..34: mol_type = other DNA
gctatactgcttaaatttggtagcatcatattgc

SEQ ID NO: 135 - protein, 4 aa, synthetic construct
AAAA

SEQ ID NO: 136 - protein, 7 aa, synthetic construct
PKKKRKV

SEQ ID NO: 137 - protein, 16 aa, synthetic construct
KRPAATKKAGQAKKKK

SEQ ID NO: 138 - protein, 9 aa, synthetic construct
PAAKRVKLD

SEQ ID NO: 139 - protein, 11 aa, synthetic construct
RQRRNELKRSP

SEQ ID NO: 140 - protein, 38 aa, synthetic construct
NQSSNFGPMKGGNFGGRSSGPYGGGGQYFAKPRNQGGY

SEQ ID NO: 141 - protein, 42 aa, synthetic construct
RMRIZFKNKGKDTAELRRRRVEVSVELRKAKKDEQILKRRNV

SEQ ID NO: 142 - protein, 8 aa, synthetic construct
VSRKRPRP

SEQ ID NO: 143 - protein, 8 aa, synthetic construct
PPKKARED

SEQ ID NO: 144 - protein, 8 aa, synthetic construct
PQPKKKPL

SEQ ID NO: 145 - protein, 12 aa, synthetic construct
SALIKKKKKMAP

SEQ ID NO: 146 - protein, 5 aa, synthetic construct
DRLRR

SEQ ID NO: 147 - protein, 7 aa, synthetic construct
PKQKKRK

SEQ ID NO: 148 - protein, 10 aa, synthetic construct
RKLKKKIKKL

SEQ ID NO: 149 - protein, 10 aa, synthetic construct
REKKKFLKRR

SEQ ID NO: 150 - protein, 20 aa, synthetic construct
KRKGDEVDGVDEVAKKKSKK

SEQ ID NO: 151 - protein, 17 aa, synthetic construct
RKCLQAGMNLEARKTKK

SEQ ID NO: 152 - protein, 17 aa, synthetic construct
RKCLQAGMNLEARKTKK

SEQ ID NO: 153 - DNA, 18 bp, synthetic construct
  source 1..18: mol_type = other DNA
caaagagaagcctcggcc

SEQ ID NO: 154 - DNA, 34 bp, synthetic construct
  source 1..34: mol_type = other DNA
gtgttaatacaaaggtacaggaacaaagaatttg

SEQ ID NO: 155 - DNA, 42 bp, synthetic construct
  source 1..42: mol_type = other DNA
gtaatttctactaagtgtagatagcattaagtgtcaagttct

SEQ ID NO: 156 - DNA, 42 bp, synthetic construct
  source 1..42: mol_type = other DNA
gtaatttctactaagtgtagatagcattaaatgtcaagttct

SEQ ID NO: 157 - DNA, 42 bp, synthetic construct
  source 1..42: mol_type = other DNA
gtaatttctactaagtgtagatactagtcccttgtactgata

SEQ ID NO: 158 - DNA, 42 bp, synthetic construct
  source 1..42: mol_type = other DNA
gtaatttctactaagtgtagatgcattctgggattctctaga

SEQ ID NO: 159 - DNA, 28 bp, synthetic construct
  source 1..28: mol_type = other DNA
ctatctgaatgaattgatttggggcttg

SEQ ID NO: 160 - DNA, 26 bp, synthetic construct
  source 1..26: mol_type = other DNA
gcaatgtcaaaatcgccattttaagc

SEQ ID NO: 161 - DNA, 22 bp, synthetic construct
  source 1..22: mol_type = other DNA
aggcaacgtccaggatagagtg

SEQ ID NO: 162 - DNA, 28 bp, synthetic construct
  source 1..28: mol_type = other DNA
cagtaagcattttccactggtatcccag

(19) United States
(12) Patent Application Publication
(10) Pub. No.: US 2025/0354212 A1
(43) Pub. Date: Nov. 20, 2025

(54) Type V CRISPR/CAS Effector Proteins for Cleaving ssDNAs and Detecting Target DNAs

(71) Applicant: The Regents of the University of California, Oakland, CA (US)

(72) Inventors: Jennifer A. Doudna, Oakland, CA (US); Janice S. Chen, Oakland, CA (US); Lucas Benjamin Harrington, Oakland, CA (US); Enbo Ma, Oakland, CA (US)

(21) Appl. No.: 19/199,204

(22) Filed: May 5, 2025

Related U.S. Application Data
(63) Continuation of application No. 17/388,321, filed on Jul. 29, 2021, now Pat. No. 12,319,963.
Application No. 17/388,321 is a continuation of application No. 16/896,731, filed on Jun. 9, 2020, now Pat. No. 11,118,224.
Application No. 16/896,731 is a continuation of application No. 16/262,257, filed on Jan. 30, 2019, now abandoned.
Application No. 16/262,257 is a continuation of application No. 15/897,089, filed on Feb. 14, 2018, now Pat. No. 10,253,365.
(60) Provisional application No. 62/590,106, filed on Nov. 22, 2017.
(60) Provisional application No. 62/626,593, filed on Feb. 5, 2018.

(51) Int. Cl. C12Q 1/6876; C12N 9/22; C12N 15/11; C12Q 1/6823

(52) U.S. Cl. CPC C12Q 1/6876; C12N 9/22; C12N 15/11; C12Q 1/6823; C12N 2310/20

(57) ABSTRACT

Provided are compositions and methods for detecting a target DNA (double stranded or single stranded) in a sample. In some embodiments, a subject method includes: (a) contacting the sample with: (i) a type V CRISPR/Cas effector protein (e.g., a Cas12 protein such as Cas12a, Cas12b, Cas12c, Cas12d, Cas12e); (ii) a guide RNA (comprising a region that binds to the type V CRISPR/Cas effector protein, and a guide sequence that hybridizes with the target DNA); and (iii) a detector DNA that is single stranded (i.e., a “single stranded detector DNA”) and does not hybridize with the guide sequence of the guide RNA; and (b) measuring a detectable signal produced by cleavage (by the type V CRISPR/Cas effector protein) of the single stranded detector DNA. Also provided are compositions and methods for cleaving single stranded DNAs (e.g., non-target ssDNAs), e.g., inside of a cell.

### CROSS-REFERENCE

This application is a continuation of U.S. patent application Ser. No. 17/388,321, filed Jul. 29, 2021, which is a continuation of U.S. patent application Ser. No. 16/896,731, filed Jun. 9, 2020, now U.S. Pat. No. 11,118,224, which is a continuation of U.S. patent application Ser. No. 16/262,257, filed Jan. 30, 2019, which is a continuation of U.S. patent application Ser. No. 15/897,089, filed Feb. 14, 2018, now U.S. Pat. No. 10,253,365, which claims the benefit of U.S. Provisional Patent Application No. 62/590,106, filed Nov. 22, 2017, and U.S. Provisional Patent Application No. 62/626,593, filed Feb. 5, 2018, which applications are incorporated herein by reference in their entirety.

### STATEMENT REGARDING FEDERALLY SPONSORED RESEARCH

This invention was made with government support under Grant No. 1244557 awarded by the National Science Foundation. The government has certain rights in the invention.

### INCORPORATION BY REFERENCE OF SEQUENCE LISTING PROVIDED AS AN XML FILE

A Sequence Listing is provided herewith as a text file, “BERK-375CON6_SEQ_LIST” created on Aug. 7, 2025 and having a size of 161,910 bytes. The contents of the XML file are incorporated by reference herein in their entirety.

### INTRODUCTION

Bacterial adaptive immune systems employ CRISPRs (clustered regularly interspaced short palindromic repeats) and CRISPR-associated (Cas) proteins for RNA-guided nucleic acid cleavage. The CRISPR-Cas systems thereby confer adaptive immunity in bacteria and archaea via RNA-guided nucleic acid interference. To provide anti-viral immunity, processed CRISPR array transcripts (crRNAs) assemble with Cas protein-containing surveillance complexes that recognize nucleic acids bearing sequence complementarity to the virus derived segment of the crRNAs, known as the spacer.

Class 2 CRISPR-Cas systems are streamlined versions in which a single Cas protein (an effector protein, e.g., a type V Cas effector protein such as Cpf1) bound to RNA is responsible for binding to and cleavage of a targeted sequence. The programmable nature of these minimal systems has facilitated their use as a versatile technology that continues to revolutionize the field of genome manipulation.

### SUMMARY

Class 2 CRISPR-Cas systems (e.g., type V CRISPR/Cas systems such as Cas12 family systems) are characterized by effector modules that include a single effector protein. For example, in a type V CRISPR/Cas system, the effector protein—a CRISPR/Cas endonuclease (e.g., a Cas12a protein)—interacts with (binds to) a corresponding guide RNA (e.g., a Cas12a guide RNA) to form a ribonucleoprotein (RNP) complex that is targeted to a particular site in a target nucleic acid via base pairing between the guide RNA and a target sequence within the target nucleic acid molecule.

The present disclosure provides compositions and methods that take advantage of the discovery that type V CRISPR/Cas proteins (e.g., Cas 12 proteins such as Cpf1 (Cas12a) and C2c1 (Cas12b)) can promiscuously cleave non-targeted single stranded DNA (ssDNA) once activated by detection of a target DNA. Once a type V CRISPR/Cas effector protein (e.g., a Cas12 protein such as Cas12a, Cas12b, Cas12c, Cas12d, Cas12e) is activated by a guide RNA, which occurs when a sample includes a target DNA to which the guide RNA hybridizes (i.e., the sample includes the targeted DNA), the protein becomes a nuclease that promiscuously cleaves ssDNAs (i.e., non-target ssDNAs, i.e., ssDNAs to which the guide sequence of the guide RNA does not hybridize). Thus, when the targeted DNA (double or single stranded) is present in the sample (e.g., in some cases above a threshold amount), the result is cleavage of ssDNAs in the sample, which can be detected using any convenient detection method (e.g., using a labeled single stranded detector DNA).

Provided are compositions and methods for detecting a target DNA (double stranded or single stranded) in a sample. In some cases, a subject method includes: (a) contacting the sample with: (i) a type V CRISPR/Cas effector protein (e.g., a Cas12 protein such as Cas12a, Cas12b, Cas12c, Cas12d, Cas12e); (ii) a guide RNA (comprising a region that binds to the type V CRISPR/Cas effector protein, and a guide sequence that hybridizes with the target DNA); and (iii) a detector DNA that is single stranded (i.e., a “single stranded detector DNA”) and does not hybridize with the guide sequence of the guide RNA; and (b) measuring a detectable signal produced by cleavage (by the type V CRISPR/Cas effector protein) of the single stranded detector DNA. In some cases, the single stranded detector DNA includes a fluorescence-emitting dye pair (e.g., a fluorescence-emitting dye pair is a fluorescence resonance energy transfer (FRET) pair, a quencher/fluor pair). In some cases, the target DNA is a viral DNA (e.g., papovavirus, hepadnavirus, herpesvirus, adenovirus, poxvirus, parvovirus, and the like).

Also provided are compositions and methods for cleaving single stranded DNAs (ssDNAs). In some cases, such methods include contacting a population of nucleic acids, wherein said population comprises a target DNA and a plurality of non-target ssDNAs, with: (i) a type V CRISPR/Cas effector protein (e.g., a Cas12 protein such as Cas12a, Cas12b, Cas12c, Cas12d, Cas12e); and (ii) a guide RNA (comprising a region that binds to the type V CRISPR/Cas effector protein, and a guide sequence that hybridizes with the target DNA), where the type V CRISPR/Cas effector protein cleaves non-target ssDNAs of said plurality. In some cases, the contacting is inside of a cell such as a eukaryotic cell, a plant cell, a mammalian cell, etc. (e.g., in vitro, ex vivo, in vivo).

Also provided are compositions (e.g., kits) for practicing the subject methods.

### BRIEF DESCRIPTION OF THE DRAWINGS

FIGS. 1A-1E provide amino acid sequences of various Type V CRISPR/Cas effector proteins (depicted are Cas12a and Cas12b sequences).

FIG. 2 provides example guide RNA sequences (e.g., crRNA repeat sequences and an example single guide RNA sequence) and example PAM sequences.

FIGS. 3A-3B present data related to non-complementary strand cleavage.

FIGS. 4A-4B present data related to non-specific DNase activity by Cas12a.

FIGS. 5A-5B present data related to non-target strand cleavage.

FIG. 6 presents data related to showing that the RuvC nuclease is responsible for trans-cleavage of ssDNA.

FIG. 7 presents data related to rapid “shredding” of M13 phage ssDNA.

FIG. 8 presents data related to detection using an FQ-based assay.

FIGS. 9A-9B present data related to mismatches at the PAM-proximal end.

FIG. 10 presents data related to turnover kinetics.

FIGS. 11A-11B present data related to distinguishing viral serotypes using a subject method of detection.

FIG. 12 presents a schematic model for DNA cleavage by CRISPR-Cas12a.

FIGS. 13A-13C present data showing that Cas12a target recognition activates non-specific single stranded DNA cleavage. FIG. 13A. Cartoon of the Cas12a-crRNA complex targeting a dsDNA substrate with cleavage sites depicting the 5′ overhang staggered cut. FIG. 13B. Timecourse of purified LbaCas12a targeting the circular, single-stranded M13 DNA phage in vitro reveals a robust shredding pattern. FIG. 13C. Timecourse of purified SpyCas9 targeting M13 ssDNA phage.

FIGS. 14A-14C present data showing that Cas12a trans-cleavage activity requires a complementary activator. FIG. 14A. Radiolabeled target dsDNA or FIG. 14B.) non-specific ssDNA incubated with molar ratios of LbaCas12a-crRNA as indicated. Each point represents quantified % cleavage after 30-minutes at 37 C, when the reaction was at completion. FIG. 14C. Michaelis-Menten kinetics of LbCas12a trans-cleavage using a dsDNA or ssDNA activator.

FIGS. 15A-15C present data showing that specificity of trans-cleavage activation involves PAM recognition and DNA unwinding. FIG. 15A. Trans-cleavage products on a denaturing PAGE gel with the indicated activators. FIG. 15B. Observed trans-cleavage rates using a ssDNA or dsDNA activator with indicated mismatches. FIG. 15C. LbaCas12a can distinguish two closely related dsDNA HPV sequences.

FIGS. 16A-16C present data showing that non-specific ssDNA cleavage activity is conserved across Type V CRISPR systems. FIG. 16A. Phylogenetic tree highlighting indicated type V effector proteins. FIG. 16B. Cleavage gels depicting activator-dependent trans-cleavage across type V effector proteins, but not the type II effector SpyCas9. FIG. 16C. Model for PAM-dependent and PAM-independent activation of cis and trans-cleavage.

FIG. 17 presents data showing that target strand recognition is a pre-requisite for single-stranded DNA cleavage.

FIGS. 18A-18C present data showing that the RuvC nuclease is responsible for non-specific DNase activity.

FIG. 19 presents data showing that the circular, single-stranded M13 DNA phage is degraded in trans by a pre-activated LbaCas12a complex.

FIGS. 20A-20B present data showing that LbaCas12a is activated by a dsDNA plasmid for trans-cleavage.

FIG. 21 presents data showing that LbaCas12a trans-cleavage degrades complementary and non-specific ssDNA, but not ssRNA.

FIG. 22 presents data showing that Michaelis-Menten kinetics reveals robust trans-cleavage activity with a ssDNA and dsDNA activator.

FIG. 23 presents data showing that the PAM sequence and PAM-proximal mismatches in a dsDNA activator provide specificity for trans-activation.

FIG. 24 presents data showing an HPV detection assay timecourse detected using a subject method of detection (e.g., labeled detector ssDNA).

FIGS. 25A-25C present data showing that Cas12a target recognition activates non-specific single-stranded DNA cleavage.

FIGS. 26A-26D present data related to kinetics of Cas12a ssDNA trans-cleavage.

FIGS. 27A-27C present data showing specificity and conservation of trans-cleavage activation.

FIGS. 28A-28D present data showing rapid identification of HPV types 16 and 18 in human samples by DETECTR.

FIG. 29 presents a schematic model for PAM-dependent and PAM-independent activation of cis and trans-cleavage by Cas12a.

FIG. 30 presents data showing purification of Cas12 and Cas9 proteins.

FIGS. 31A-31B present data showing that LbCas12a is a DNA-activated general DNase.

FIGS. 32A-32B present data showing that target strand recognition is a pre-requisite for single-stranded DNA cleavage

FIGS. 33A-33C present data showing that the RuvC nuclease domain is responsible for activator-dependent, non-specific DNase activity.

FIGS. 34A-34C present data showing that LbCas12a trans-cleavage degrades complementary and non-specific ssDNA, but not ssRNA.

FIGS. 35A-35B present data showing that target strand cleavage by Cas12a is not required for triggering non-specific ssDNase activity.

FIGS. 36A-36E present data showing Michaelis-Menten analysis that reveals robust trans-cleavage activity with a ssDNA and dsDNA activator.

FIG. 37 presents data showing that PAM sequence and PAM-proximal mismatches in a dsDNA activator provide specificity for trans-activation.

FIG. 38 presents data showing that activator-dependent, non-specific ssDNA cleavage activity is conserved across type V CRISPR interference proteins.

FIGS. 39A-39E present data showing that Cas12a can distinguish two closely related HPV sequences.

FIGS. 40A-40B present data showing that isothermal amplification coupled with Cas12a detection yields DETECTR, which can achieve attomolar sensitivity

FIGS. 41A-41D present data showing identification of HPV types 16 and 18 in human cell lines and patient samples by DETECTR

FIGS. 42A-42B present data showing PCR and hybrid capture validation and genotyping of HPV in human clinical samples.

FIGS. 43A-43B present data showing identification of target nucleic acid by DETECTR using Cas12d and Cas12e proteins.

FIG. 44 presents data showing identification of a single nucleotide polymorphism (SNP) within the HERC2 gene responsible for brown or blue eyes using DETECTR.

FIG. 45 presents data showing identification of the X or Y chromosomes through detection of the XIST (within X chromosome) or SRY (within Y chromosome) genes from human saliva (using the DETECTR assay).

FIG. 46 presents a schematic illustrating DETECTR as a platform for rapid, point-of-care diagnostics.

### DEFINITIONS

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

The term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized. The terms “polynucleotide” and “nucleic acid” should be understood to include, as applicable to the embodiments being described, single-stranded (such as sense or antisense) and double-stranded polynucleotides.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, Phyre2, etc.), available over the world wide web at sites including ncbi(dot)nlm(dot)nili(dot)gov/BLAST, ebi(dot)ac(dot)uk/Tools/msa/tcoffee/, ebi(dot)ac(dot)uk/Tools/msa/muscle/, mafft(dot)cbrc(dot)jp/alignment/software/, sbg(dot)bio(dot)ic(dot)ac(dot)uk/˜phyre2/. See, e.g., Altschul et al. (1990), J. Mol. Bioi. 215:403-10.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., protein coding) and/or regulate translation of an encoded polypeptide.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Any given component, or combination of components can be unlabeled, or can be detectably labeled with a label moiety. In some cases, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a type V CRISPR/Cas effector protein” includes a plurality of such type V CRISPR/Cas effector proteins and reference to “the guide RNA” includes reference to one or more guide RNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As noted above, the inventors have discovered that that type V CRISPR/Cas proteins, e.g., Cas12 proteins such as Cpf1 (Cas12a) and C2c1 (Cas12b) can promiscuously cleave non-targeted single stranded DNA (ssDNA) once activated by detection of a target DNA (double or single stranded). Once a type V CRISPR/Cas effector protein (e.g., a Cas12 protein such as Cas12a, Cas12b, Cas12c, Cas12d, Cas12e) is activated by a guide RNA, which occurs when the guide RNA hybridizes to a target sequence of a target DNA (i.e., the sample includes the targeted DNA), the protein becomes a nuclease that promiscuously cleaves ssDNAs (i.e., the nuclease cleaves non-target ssDNAs, i.e., ssDNAs to which the guide sequence of the guide RNA does not hybridize). Thus, when the target DNA is present in the sample (e.g., in some cases above a threshold amount), the result is cleavage of ssDNAs in the sample, which can be detected using any convenient detection method (e.g., using a labeled single stranded detector DNA).

Provided are compositions and methods for detecting a target DNA (double stranded or single stranded) in a sample. In some cases, a detector DNA is used that is single stranded (ssDNA) and does not hybridize with the guide sequence of the guide RNA (i.e., the detector ssDNA is a non-target ssDNA). Such methods can include (a) contacting the sample with: (i) a type V CRISPR/Cas effector protein (e.g., a Cas12 protein); (ii) a guide RNA comprising: a region that binds to the type V CRISPR/Cas effector protein, and a guide sequence that hybridizes with the target DNA; and (iii) a detector DNA that is single stranded and does not hybridize with the guide sequence of the guide RNA; and (b) measuring a detectable signal produced by cleavage of the single stranded detector DNA by the type V CRISPR/Cas effector protein, thereby detecting the target DNA. As noted above, once a subject Type V CRISPR/Cas effector protein (e.g., a Cas12 protein such as Cas12a, Cas12b, Cas12c, Cas12d, Cas12e) is activated by a guide RNA, which occurs when the sample includes a target DNA to which the guide RNA hybridizes (i.e., the sample includes the targeted target DNA), the Type V CRISPR/Cas effector protein (e.g., a Cas12 protein such as Cas12a, Cas12b, Cas12c, Cas12d, Cas12e) is activated and functions as an endoribonuclease that non-specifically cleaves ssDNAs (including non-target ssDNAs) present in the sample. Thus, when the targeted target DNA is present in the sample (e.g., in some cases above a threshold amount), the result is cleavage of ssDNA (including non-target ssDNA) in the sample, which can be detected using any convenient detection method (e.g., using a labeled detector ssDNA).

Also provided are compositions and methods for cleaving single stranded DNAs (ssDNAs) (e.g., non-target ssDNAs). Such methods can include contacting a population of nucleic acids, wherein said population comprises a target DNA and a plurality of non-target ssDNAs, with: (i) a type V CRISPR/Cas effector protein; and (ii) a guide RNA comprising: a region that binds to the type V CRISPR/Cas effector protein, and a guide sequence that hybridizes with the target DNA, wherein the type V CRISPR/Cas effector protein cleaves non-target ssDNAs of said plurality. Such a method can be used, e.g., to cleave foreign ssDNAs (e.g., viral DNAs) in a cell.

The contacting step of a subject method can be carried out in a composition comprising divalent metal ions. The contacting step can be carried out in an acellular environment, e.g., outside of a cell. The contacting step can be carried out inside a cell. The contacting step can be carried out in a cell in vitro. The contacting step can be carried out in a cell ex vivo. The contacting step can be carried out in a cell in vivo.

The guide RNA can be provided as RNA or as a nucleic acid encoding the guide RNA (e.g., a DNA such as a recombinant expression vector). The Type V CRISPR/Cas effector protein (e.g., a Cas12 protein such as Cas12a, Cas12b, Cas12c, Cas12d, Cas12e) can be provided as a protein or as a nucleic acid encoding the protein (e.g., an mRNA, a DNA such as a recombinant expression vector). In some cases, two or more (e.g., 3 or more, 4 or more, 5 or more, or 6 or more) guide RNAs can be provided by (e.g., using a precursor guide RNA array, which can be cleaved by the Type V CRISPR/Cas effector protein into individual (“mature”) guide RNAs).

In some cases (e.g., when contacting with a guide RNA and a Type V CRISPR/Cas effector protein (e.g., a Cas12 protein such as Cas12a, Cas12b, Cas12c, Cas12d, Cas12e)), the sample is contacted for 2 hours or less (e.g., 1.5 hours or less, 1 hour or less, 40 minutes or less, 30 minutes or less, 20 minutes or less, 10 minutes or less, or 5 minutes or less, or 1 minute or less) prior to the measuring step. For example, in some cases the sample is contacted for 40 minutes or less prior to the measuring step. In some cases the sample is contacted for 20 minutes or less prior to the measuring step. In some cases the sample is contacted for 10 minutes or less prior to the measuring step. In some cases the sample is contacted for 5 minutes or less prior to the measuring step. In some cases the sample is contacted for 1 minute or less prior to the measuring step. In some cases the sample is contacted for from 50 seconds to 60 seconds prior to the measuring step. In some cases the sample is contacted for from 40 seconds to 50 seconds prior to the measuring step. In some cases the sample is contacted for from 30 seconds to 40 seconds prior to the measuring step. In some cases the sample is contacted for from 20 seconds to 30 seconds prior to the measuring step. In some cases the sample is contacted for from 10 seconds to 20 seconds prior to the measuring step.

A method of the present disclosure for detecting a target DNA (single-stranded or double-stranded) in a sample can detect a target DNA with a high degree of sensitivity. In some cases, a method of the present disclosure can be used to detect a target DNA present in a sample comprising a plurality of DNAs (including the target DNA and a plurality of non-target DNAs), where the target DNA is present at one or more copies per 10⁷ non-target DNAs (e.g., one or more copies per 10⁶ non-target DNAs, one or more copies per 10⁵ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs). In some cases, a method of the present disclosure can be used to detect a target DNA present in a sample comprising a plurality of DNAs (including the target DNA and a plurality of non-target DNAs), where the target DNA is present at one or more copies per 10¹⁸ non-target DNAs (e.g., one or more copies per 10¹⁵ non-target DNAs, one or more copies per 10¹² non-target DNAs, one or more copies per 10⁹ non-target DNAs, one or more copies per 10⁶ non-target DNAs, one or more copies per 10⁵ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs).

In some cases, a method of the present disclosure can detect a target DNA present in a sample, where the target DNA is present at from one copy per 10⁷ non-target DNAs to one copy per 10 non-target DNAs (e.g., from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 10 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 10 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, a method of the present disclosure can detect a target DNA present in a sample, where the target DNA is present at from one copy per 10¹⁸ non-target DNAs to one copy per 10 non-target DNAs (e.g., from 1 copy per 10¹⁸ non-target DNAs to 1 copy per 10² non-target DNAs, from 1 copy per 10¹⁵ non-target DNAs to 1 copy per 10² non-target DNAs, from 1 copy per 10¹² non-target DNAs to 1 copy per 10² non-target DNAs, from 1 copy per 10⁹ non-target DNAs to 1 copy per 10² non-target DNAs, from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 10 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 10 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, a method of the present disclosure can detect a target DNA present in a sample, where the target DNA is present at from one copy per 10⁷ non-target DNAs to one copy per 100 non-target DNAs (e.g., from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 100 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 100 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10² non-target DNAs to 1 copy per 10⁴ non-target DNAs).

In some cases, the threshold of detection, for a subject method of detecting a target DNA in a sample, is 10 nM or less. The term “threshold of detection” is used herein to describe the minimal amount of target DNA that must be present in a sample in order for detection to occur. Thus, as an illustrative example, when a threshold of detection is 10 nM, then a signal can be detected when a target DNA is present in the sample at a concentration of 10 nM or more. In some cases, a method of the present disclosure has a threshold of detection of 5 nM or less. In some cases, a method of the present disclosure has a threshold of detection of 1 nM or less. In some cases, a method of the present disclosure has a threshold of detection of 0.5 nM or less. In some cases, a method of the present disclosure has a threshold of detection of 0.1 nM or less. In some cases, a method of the present disclosure has a threshold of detection of 0.05 nM or less. In some cases, a method of the present disclosure has a threshold of detection of 0.01 nM or less. In some cases, a method of the present disclosure has a threshold of detection of 0.005 nM or less. In some cases, a method of the present disclosure has a threshold of detection of 0.001 nM or less. In some cases, a method of the present disclosure has a threshold of detection of 0.0005 nM or less. In some cases, a method of the present disclosure has a threshold of detection of 0.0001 nM or less. In some cases, a method of the present disclosure has a threshold of detection of 0.00005 nM or less. In some cases, a method of the present disclosure has a threshold of detection of 0.00001 nM or less. In some cases, a method of the present disclosure has a threshold of detection of 10 pM or less. In some cases, a method of the present disclosure has a threshold of detection of 1 pM or less. In some cases, a method of the present disclosure has a threshold of detection of 500 fM or less. In some cases, a method of the present disclosure has a threshold of detection of 250 fM or less. In some cases, a method of the present disclosure has a threshold of detection of 100 fM or less. In some cases, a method of the present disclosure has a threshold of detection of 50 fM or less. In some cases, a method of the present disclosure has a threshold of detection of 500 aM (attomolar) or less. In some cases, a method of the present disclosure has a threshold of detection of 250 aM or less. In some cases, a method of the present disclosure has a threshold of detection of 100 aM or less. In some cases, a method of the present disclosure has a threshold of detection of 50 aM or less. In some cases, a method of the present disclosure has a threshold of detection of 10 aM or less. In some cases, a method of the present disclosure has a threshold of detection of 1 aM or less.

In some cases, the threshold of detection (for detecting the target DNA in a subject method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target DNA at which the target DNA can be detected). In some cases, a method of the present disclosure has a threshold of detection in a range of from 800 fM to 100 pM. In some cases, a method of the present disclosure has a threshold of detection in a range of from 1 pM to 10 pM. In some cases, a method of the present disclosure has a threshold of detection in a range of from 10 fM to 500 fM, e.g., from 10 fM to 50 fM, from 50 fM to 100 fM, from 100 fM to 250 fM, or from 250 fM to 500 fM.

In some cases, the minimum concentration at which a target DNA can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target DNA can be detected in a sample is in a range of from 800 fM to 100 pM. In some cases, the minimum concentration at which a target DNA can be detected in a sample is in a range of from 1 pM to 10 pM.

In some cases, the threshold of detection (for detecting the target DNA in a subject method), is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target DNA at which the target DNA can be detected). In some cases, a method of the present disclosure has a threshold of detection in a range of from 1 aM to 800 aM. In some cases, a method of the present disclosure has a threshold of detection in a range of from 50 aM to 1 pM. In some cases, a method of the present disclosure has a threshold of detection in a range of from 50 aM to 500 fM.

In some cases, the minimum concentration at which a target DNA can be detected in a sample is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target DNA can be detected in a sample is in a range of from 1 aM to 500 pM. In some cases, the minimum concentration at which a target DNA can be detected in a sample is in a range of from 100 aM to 500 pM.

In some cases, a subject composition or method exhibits an attomolar (aM) sensitivity of detection. In some cases, a subject composition or method exhibits a femtomolar (fM) sensitivity of detection. In some cases, a subject composition or method exhibits a picomolar (pM) sensitivity of detection. In some cases, a subject composition or method exhibits a nanomolar (nM) sensitivity of detection.

### Target DNA

A target DNA can be single stranded (ssDNA) or double stranded (dsDNA). When the target DNA is single stranded, there is no preference or requirement for a PAM sequence in the target DNA. However, when the target DNA is dsDNA, a PAM is usually present adjacent to the target sequence of the target DNA (e.g., see discussion of the PAM elsewhere herein). The source of the target DNA can be the same as the source of the sample, e.g., as described below.

The source of the target DNA can be any source. In some cases the target DNA is a viral DNA (e.g., a genomic DNA of a DNA virus). As such, subject method can be for detecting the presence of a viral DNA amongst a population of nucleic acids (e.g., in a sample). A subject method can also be used for the cleavage of non-target ssDNAs in the present of a target DNA. For example, if a method takes place in a cell, a subject method can be used to promiscuously cleave non-target ssDNAs in the cell (ssDNAs that do not hybridize with the guide sequence of the guide RNA) when a particular target DNA is present in the cell (e.g., when the cell is infected with a virus and viral target DNA is detected).

Examples of possible target DNAs include, but are not limited to, viral DNAs such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. In some cases, the target DNA is parasite DNA. In some cases, the target DNA is bacterial DNA, e.g., DNA of a pathogenic bacterium.

### Samples

A subject sample includes nucleic acid (e.g., a plurality of nucleic acids). The term “plurality” is used herein to mean two or more. Thus, in some cases a sample includes two or more (e.g., 3 or more, 5 or more, 10 or more, 20 or more, 50 or more, 100 or more, 500 or more, 1,000 or more, or 5,000 or more) nucleic acids (e.g., DNAs). A subject method can be used as a very sensitive way to detect a target DNA present in a sample (e.g., in a complex mixture of nucleic acids such as DNAs). In some cases the sample includes 5 or more DNAs (e.g., 10 or more, 20 or more, 50 or more, 100 or more, 500 or more, 1,000 or more, or 5,000 or more DNAs) that differ from one another in sequence. In some cases, the sample includes 10 or more, 20 or more, 50 or more, 100 or more, 500 or more, 10³ or more, 5×10³ or more, 10⁴ or more, 5×10⁴ or more, 10⁵ or more, 5×10⁵ or more, 10⁶ or more 5×10⁶ or more, or 10⁷ or more, DNAs. In some cases, the sample comprises from 10 to 20, from 20 to 50, from 50 to 100, from 100 to 500, from 500 to 10³, from 10³ to 5×10³, from 5×10³ to 10⁴, from 10⁴ to 5×10⁴, from 5×10⁴ to 10⁵, from 10⁵ to 5×10⁵, from 5×10⁵ to 10⁶, from 10⁶ to 5×10⁶, or from 5×10⁶ to 10⁷, or more than 10⁷, DNAs. In some cases, the sample comprises from 5 to 10⁷ DNAs (e.g., that differ from one another in sequence)(e.g., from 5 to 10⁶, from 5 to 10⁵, from 5 to 50,000, from 5 to 30,000, from 10 to 10⁶, from 10 to 10⁵, from 10 to 50,000, from 10 to 30,000, from 20 to 10⁶, from 20 to 10⁵, from 20 to 50,000, or from 20 to 30,000 DNAs). In some cases the sample includes 20 or more DNAs that differ from one another in sequence. In some cases, the sample includes DNAs from a cell lysate (e.g., a eukaryotic cell lysate, a mammalian cell lysate, a human cell lysate, a prokaryotic cell lysate, a plant cell lysate, and the like). For example, in some cases the sample includes DNA from a cell such as a eukaryotic cell, e.g., a mammalian cell such as a human cell.

The term “sample” is used herein to mean any sample that includes DNA (e.g., in order to determine whether a target DNA is present among a population of DNAs). The sample can be derived from any source, e.g., the sample can be a synthetic combination of purified DNAs; the sample can be a cell lysate, an DNA-enriched cell lysate, or DNAs isolated and/or purified from a cell lysate. The sample can be from a patient (e.g., for the purpose of diagnosis). The sample can be from permeabilized cells. The sample can be from crosslinked cells. The sample can be in tissue sections. The sample can be from tissues prepared by crosslinking followed by delipidation and adjustment to make a uniform refractive index. Examples of tissue preparation by crosslinking followed by delipidation and adjustment to make a uniform refractive index have been described in, for example, Shah et al., Development (2016) 143, 2862-2867 doi:10.1242/dev.138560.

A “sample” can include a target DNA and a plurality of non-target DNAs. In some cases, the target DNA is present in the sample at one copy per 10 non-target DNAs, one copy per 20 non-target DNAs, one copy per 25 non-target DNAs, one copy per 50 non-target DNAs, one copy per 100 non-target DNAs, one copy per 500 non-target DNAs, one copy per 10³ non-target DNAs, one copy per 5×10³ non-target DNAs, one copy per 10⁴ non-target DNAs, one copy per 5×10⁴ non-target DNAs, one copy per 10⁵ non-target DNAs, one copy per 5×10⁵ non-target DNAs, one copy per 10⁶ non-target DNAs, or less than one copy per 10⁶ non-target DNAs. In some cases, the target DNA is present in the sample at from one copy per 10 non-target DNAs to 1 copy per 20 non-target DNAs, from 1 copy per 20 non-target DNAs to 1 copy per 50 non-target DNAs, from 1 copy per 50 non-target DNAs to 1 copy per 100 non-target DNAs, from 1 copy per 100 non-target DNAs to 1 copy per 500 non-target DNAs, from 1 copy per 500 non-target DNAs to 1 copy per 10³ non-target DNAs, from 1 copy per 10³ non-target DNAs to 1 copy per 5×10³ non-target DNAs, from 1 copy per 5×10³ non-target DNAs to 1 copy per 10⁴ non-target DNAs, from 1 copy per 10⁴ non-target DNAs to 1 copy per 10⁵ non-target DNAs, from 1 copy per 10⁵ non-target DNAs to 1 copy per 10⁶ non-target DNAs, or from 1 copy per 10⁶ non-target DNAs to 1 copy per 10⁷ non-target DNAs.

Suitable samples include but are not limited to saliva, blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., DNAs. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNAs that is obtained from such cells (e.g., a cell lysate or other cell extract comprising DNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell, tissue, or organ; a cell, tissue, or organ from an invertebrate animal (e.g. fruit fly, cnidarian, echinoderm, nematode, an insect, an arachnid, etc.); a cell, tissue, fluid, or organ from a vertebrate animal (e.g., fish, amphibian, reptile, bird, mammal); a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms, e.g., Bacteria (e.g., Eubacteria); Archaebacteria; Protista; Fungi; Plantae; and Animalia. Suitable sample sources include plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable sample sources include include members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable sample sources include include members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable sample sources include include members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plants include any monocotyledon and any dicotyledon.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some cases, the source of the sample is a (or is suspected of being a diseased cell, fluid, tissue, or organ. In some cases, the source of the sample is a normal (non-diseased) cell, fluid, tissue, or organ. In some cases, the source of the sample is a (or is suspected of being a pathogen-infected cell, tissue, or organ. For example, the source of a sample can be an individual who may or may not be infected—and the sample could be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some cases, the sample is a cell-free liquid sample. In some cases, the sample is a liquid sample that can comprise cells. Pathogens include viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNAviruses [e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

### Measuring a Detectable Signal

In some cases, a subject method includes a step of measuring (e.g., measuring a detectable signal produced by Type V CRISPR/Cas effector protein (e.g., a Cas12 protein such as Cas12a, Cas12b, Cas12c, Cas12d, Cas12e)-mediated ssDNA cleavage). Because a Type V CRISPR/Cas effector protein (e.g., a Cas12 protein such as Cas12a, Cas12b, Cas12c, Cas12d, Cas12e) cleaves non-targeted ssDNA once activated, which occurs when a guide RNA hybridizes with a target DNA in the presence of a Type V CRISPR/Cas effector protein (e.g., a Cas12 protein such as Cas12a, Cas12b, Cas12c, Cas12d, Cas12e), a detectable signal can be any signal that is produced when ssDNA is cleaved. For example, in some cases the step of measuring can include one or more of: gold nanoparticle based detection (e.g., see Xu et al., Angew Chem Int Ed Engl. 2007; 46(19):3468-70; and Xia et al., Proc Natl Acad Sci USA. 2010 Jun. 15; 107(24):10837-41), fluorescence polarization, colloid phase transition/dispersion (e.g., Baksh et al., Nature. 2004 Jan. 8; 427(6970):139-41), electrochemical detection, semiconductor-based sensing (e.g., Rothberg et al., Nature. 2011 Jul. 20; 475(7356):348-52; e.g., one could use a phosphatase to generate a pH change after ssDNA cleavage reactions, by opening 2′-3′ cyclic phosphates, and by releasing inorganic phosphate into solution), and detection of a labeled detector ssDNA (see elsewhere herein for more details). The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

The measuring can in some cases be quantitative, e.g., in the sense that the amount of signal detected can be used to determine the amount of target DNA present in the sample. The measuring can in some cases be qualitative, e.g., in the sense that the presence or absence of detectable signal can indicate the presence or absence of targeted DNA (e.g., virus, SNP, etc.). In some cases, a detectable signal will not be present (e.g., above a given threshold level) unless the targeted DNA(s) (e.g., virus, SNP, etc.) is present above a particular threshold concentration. In some cases, the threshold of detection can be titrated by modifying the amount of Type V CRISPR/Cas effector protein (e.g., a Cas12 protein such as Cas12a, Cas12b, Cas12c, Cas12d, Cas12e), guide RNA, sample volume, and/or detector ssDNA (if one is used). As such, for example, as would be understood by one of ordinary skill in the art, a number of controls can be used if desired in order to set up one or more reactions, each set up to detect a different threshold level of target DNA, and thus such a series of reactions could be used to determine the amount of target DNA present in a sample (e.g., one could use such a series of reactions to determine that a target DNA is present in the sample ‘at a concentration of at least X’). Non-limiting examples of applications of/uses for the compositions and methods of the disclosure include those depicted in FIG. 46. The figure depicts embodiments in which nucleic acids of the sample are amplified (denoted as “RPA” in FIG. 46 prior to contact with a Cas12 protein, but the same applications/uses (e.g., SNP detection, cancer screening, detection of bacterial infection, detection of antibiotic resistance, detection of viral infection, and the like) can apply to embodiments in which no amplification step is included. The compositions and methods of this disclosure can be used to detect any DNA target. For example, any virus that integrates nucleic acid material into the genome can be detected because a subject sample can include cellular genomic DNA—and the guide RNA can be designed to detect integrated nucleotide sequence.

In some cases, a method of the present disclosure can be used to determine the amount of a target DNA in a sample (e.g., a sample comprising the target DNA and a plurality of non-target DNAs). Determining the amount of a target DNA in a sample can comprise comparing the amount of detectable signal generated from a test sample to the amount of detectable signal generated from a reference sample. Determining the amount of a target DNA in a sample can comprise: measuring the detectable signal to generate a test measurement; measuring a detectable signal produced by a reference sample to generate a reference measurement; and comparing the test measurement to the reference measurement to determine an amount of target DNA present in the sample.

For example, in some cases, a method of the present disclosure for determining the amount of a target DNA in a sample comprises: a) contacting the sample (e.g., a sample comprising the target DNA and a plurality of non-target DNAs) with: (i) a guide RNA that hybridizes with the target DNA, (ii) a Type V CRISPR/Cas effector protein (e.g., a Cas12 protein such as Cas12a, Cas12b, Cas12c, Cas12d, Cas12e) that cleaves RNAs present in the sample, and (iii) a detector ssDNA; b) measuring a detectable signal produced by Type V CRISPR/Cas effector protein (e.g., a Cas12 protein such as Cas12a, Cas12b, Cas12c, Cas12d, Cas12e)-mediated ssDNA cleavage (e.g., cleavage of the detector ssDNA), generating a test measurement; c) measuring a detectable signal produced by a reference sample to generate a reference measurement; and d) comparing the test measurement to the reference measurement to determine an amount of target DNA present in the sample.

As another example, in some cases, a method of the present disclosure for determining the amount of a target DNA in a sample comprises: a) contacting the sample (e.g., a sample comprising the target DNA and a plurality of non-target DNAs) with: i) a precursor guide RNA array comprising two or more guide RNAs each of which has a different guide sequence; (ii) a Type V CRISPR/Cas effector protein (e.g., a Cas12 protein such as Cas12a, Cas12b, Cas12c, Cas12d, Cas12e) that cleaves the precursor guide RNA array into individual guide RNAs, and also cleaves RNAs of the sample; and (iii) a detector ssDNA; b) measuring a detectable signal produced by Type V CRISPR/Cas effector protein (e.g., a Cas12 protein such as Cas12a, Cas12b, Cas12c, Cas12d, Cas12e)-mediated ssDNA cleavage (e.g., cleavage of the detector ssDNA), generating a test measurement; c) measuring a detectable signal produced by each of two or more reference samples to generate two or more reference measurements; and d) comparing the test measurement to the reference measurements to determine an amount of target DNA present in the sample.

### Amplification of Nucleic Acids in the Sample

In some embodiments, sensitivity of a subject composition and/or method (e.g., for detecting the presence of a target DNA, such as viral DNA or a SNP, in cellular genomic DNA) can be increased by coupling detection with nucleic acid amplification. In some cases, the nucleic acids in a sample are amplified prior to contact with a type V CRISPR/Cas effector protein (e.g., a Cas12 protein) that cleaved ssDNA (e.g., amplification of nucleic acids in the sample can begin prior to contact with a type V CRISPR/Cas effector protein). In some cases, the nucleic acids in a sample are amplified simultaneous with contact with a type V CRISPR/Cas effector protein (e.g., a Cas12 protein). For example, in some cases a subject method includes amplifying nucleic acids of a sample (e.g., by contacting the sample with amplification components) prior to contacting the amplified sample with a type V CRISPR/Cas effector protein (e.g., a Cas12 protein). In some cases a subject method includes contacting a sample with amplification components at the same time (simultaneous with) that the sample is contacted with a type V CRISPR/Cas effector protein (e.g., a Cas12 protein). If all components are added simultaneously (amplification components and detection components such as a type V CRISPR/Cas effector protein, e.g., a Cas12 protein, a guide RNA, and a detector DNA), it is possible that the trans-cleavage activity of the type V CRISPR/Cas effector protein (e.g., a Cas12 protein), will begin to degrade the nucleic acids of the sample at the same time the nucleic acids are undergoing amplification. However, even if this is the case, amplifying and detecting simultaneously can still increase sensitivity compared to performing the method without amplification.

In some cases specific sequences (e.g., sequences of a virus, sequences that include a SNP of interest) are amplified from the sample, e.g., using primers. As such, a sequence to which the guide RNA will hybridize can be amplified in order to increase sensitivity of a subject detection method—this could achieve biased amplification of a desired sequence in order to increase the number of copies of the sequence of interest present in the sample relative to other sequences present in the sample. As one illustrative example, if a subject method is being used to determine whether a given sample includes a particular virus (or a particular SNP), a desired region of viral sequence (or non-viral genomic sequence) can be amplified, and the region amplified will include the sequence that would hybridize to the guide RNA if the viral sequence (or SNP) were in fact present in the sample.

As noted, in some cases the nucleic acids are amplified (e.g., by contact with amplification components) prior to contacting the amplified nucleic acids with a type V CRISPR/Cas effector protein (e.g., a Cas12 protein). In some cases, amplification occurs for 10 seconds or more, (e.g., 30 seconds or more, 45 seconds or more, 1 minute or more, 2 minutes or more, 3 minutes or more, 4 minutes or more, 5 minutes or more, 7.5 minutes or more, 10 minutes or more, etc.) prior to contact with an active type V CRISPR/Cas effector protein (e.g., a Cas12 protein). In some cases, amplification occurs for 2 minutes or more (e.g., 3 minutes or more, 4 minutes or more, 5 minutes or more, 7.5 minutes or more, 10 minutes or more, etc.) prior to contact with an active type V CRISPR/Cas effector protein (e.g., a Cas12 protein). In some cases, amplification occurs for a period of time in a range of from 10 seconds to 60 minutes (e.g., 10 seconds to 40 minutes, 10 seconds to 30 minutes, 10 seconds to 20 minutes, 10 seconds to 15 minutes, 10 seconds to 10 minutes, 10 seconds to 5 minutes, 30 seconds to 40 minutes, 30 seconds to 30 minutes, 30 seconds to 20 minutes, 30 seconds to 15 minutes, 30 seconds to 10 minutes, 30 seconds to 5 minutes, 1 minute to 40 minutes, 1 minute to 30 minutes, 1 minute to 20 minutes, 1 minute to 15 minutes, 1 minute to 10 minutes, 1 minute to 5 minutes, 2 minutes to 40 minutes, 2 minutes to 30 minutes, 2 minutes to 20 minutes, 2 minutes to 15 minutes, 2 minutes to 10 minutes, 2 minutes to 5 minutes, 5 minutes to 40 minutes, 5 minutes to 30 minutes, 5 minutes to 20 minutes, 5 minutes to 15 minutes, or 5 minutes to 10 minutes). In some cases, amplification occurs for a period of time in a range of from 5 minutes to 15 minutes. In some cases, amplification occurs for a period of time in a range of from 7 minutes to 12 minutes.

In some cases, a sample is contacted with amplification components at the same time as contact with a type V CRISPR/Cas effector protein (e.g., a Cas12 protein). In some such cases, the type V CRISPR/Cas effector protein in inactive at the time of contact and is activated once nucleic acids in the sample have been amplified.

Various amplification methods and components will be known to one of ordinary skill in the art and any convenient method can be used (see, e.g., Zanoli and Spoto, Biosensors (Basel). 2013 March; 3(1): 18-43; Gill and Ghaemi, Nucleosides, Nucleotides, and Nucleic Acids, 2008, 27: 224-243; Craw and Balachandrana, Lab Chip, 2012, 12, 2469-2486; which are herein incorporated by reference in their entirety). Nucleic acid amplification can comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some cases the amplification is isothermal amplification. The term “isothermal amplification” indicates a method of nucleic acid (e.g., DNA) amplification (e.g., using enzymatic chain reaction) that can use a single temperature incubation thereby obviating the need for a thermal cycler. Isothermal amplification is a form of nucleic acid amplification which does not rely on the thermal denaturation of the target nucleic acid during the amplification reaction and hence may not require multiple rapid changes in temperature. Isothermal nucleic acid amplification methods can therefore be carried out inside or outside of a laboratory environment. By combining with a reverse transcription step, these amplification methods can be used to isothermally amplify RNA.

Examples of isothermal amplification methods include but are not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

In some cases, the amplification is recombinase polymerase amplification (RPA) (see, e.g., U.S. Pat. Nos. 8,030,000; 8,426,134; 8,945,845; 9,309,502; and 9,663,820, which are hereby incorporated by reference in their entirety). Recombinase polymerase amplification (RPA) uses two opposing primers (much like PCR) and employs three enzymes—a recombinase, a single-stranded DNA-binding protein (SSB) and a strand-displacing polymerase. The recombinase pairs oligonucleotide primers with homologous sequence in duplex DNA, SSB binds to displaced strands of DNA to prevent the primers from being displaced, and the strand displacing polymerase begins DNA synthesis where the primer has bound to the target DNA. Adding a reverse transcriptase enzyme to an RPA reaction can facilitate detection RNA as well as DNA, without the need for a separate step to produce cDNA. One example of components for an RPA reaction is as follows (see, e.g., U.S. Pat. Nos. 8,030,000; 8,426,134; 8,945,845; 9,309,502; 9,663,820): 50 mM Tris pH 8.4, 80 mM Potassium actetate, 10 mM Magnesium acetate, 2 mM DTT, 5% PEG compound (Carbowax-20M), 3 mM ATP, 30 mM Phosphocreatine, 100 ng/μl creatine kinase, 420 ng/μl gp32, 140 ng/μl UvsX, 35 ng/μl UvsY, 2000M dNTPs, 300 nM each oligonucleotide, 35 ng/μl Bsu polymerase, and a nucleic acid-containing sample).

In a transcription mediated amplification (TMA), an RNA polymerase is used to make RNA from a promoter engineered in the primer region, and then a reverse transcriptase synthesizes cDNA from the primer. A third enzyme, e.g., Rnase H can then be used to degrade the RNA target from cDNA without the heat-denatured step. This amplification technique is similar to Self-Sustained Sequence Replication (3SR) and Nucleic Acid Sequence Based Amplification (NASBA), but varies in the enzymes employed. For another example, helicase-dependent amplification (HDA) utilizes a thermostable helicase (Tte-UvrD) rather than heat to unwind dsDNA to create single-strands that are then available for hybridization and extension of primers by polymerase. For yet another example, a loop mediated amplification (LAMP) employs a thermostable polymerase with strand displacement capabilities and a set of four or more specific designed primers. Each primer is designed to have hairpin ends that, once displaced, snap into a hairpin to facilitate self-priming and further polymerase extension. In a LAMP reaction, though the reaction proceeds under isothermal conditions, an initial heat denaturation step is required for double-stranded targets. In addition, amplification yields a ladder pattern of various length products. For yet another example, a strand displacement amplification (SDA) combines the ability of a restriction endonuclease to nick the unmodified strand of its target DNA and an exonuclease-deficient DNA polymerase to extend the 3′ end at the nick and displace the downstream DNA strand.

### Detector DNA

In some cases, a subject method includes contacting a sample (e.g., a sample comprising a target DNA and a plurality of non-target ssDNAs) with: i) a Type V CRISPR/Cas effector protein (e.g., a Cas12 protein such as Cas12a, Cas12b, Cas12c, Cas12d, Cas12e); ii) a guide RNA (or precursor guide RNA array); and iii) a detector DNA that is single stranded and does not hybridize with the guide sequence of the guide RNA. For example, in some cases, a subject method includes contacting a sample with a labeled single stranded detector DNA (detector ssDNA) that includes a fluorescence-emitting dye pair; the Type V CRISPR/Cas effector protein (e.g., a Cas12 protein such as Cas12a, Cas12b, Cas12c, Cas12d, Cas12e) cleaves the labeled detector ssDNA after it is activated (by binding to the guide RNA in the context of the guide RNA hybridizing to a target DNA); and the detectable signal that is measured is produced by the fluorescence-emitting dye pair. For example, in some cases, a subject method includes contacting a sample with a labeled detector ssDNA comprising a fluorescence resonance energy transfer (FRET) pair or a quencher/fluor pair, or both. In some cases, a subject method includes contacting a sample with a labeled detector ssDNA comprising a FRET pair. In some cases, a subject method includes contacting a sample with a labeled detector ssDNA comprising a fluor/quencher pair.

Fluorescence-emitting dye pairs comprise a FRET pair or a quencher/fluor pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

In some cases (e.g., when the detector ssDNA includes a FRET pair) the labeled detector ssDNA produces an amount of detectable signal prior to being cleaved, and the amount of detectable signal that is measured is reduced when the labeled detector ssDNA is cleaved. In some cases, the labeled detector ssDNA produces a first detectable signal prior to being cleaved (e.g., from a FRET pair) and a second detectable signal when the labeled detector ssDNA is cleaved (e.g., from a quencher/fluor pair). As such, in some cases, the labeled detector ssDNA comprises a FRET pair and a quencher/fluor pair.

In some cases, the labeled detector ssDNA comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Förster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”.

The donor-acceptor pair (a FRET donor moiety and a FRET acceptor moiety) is referred to herein as a “FRET pair” or a “signal FRET pair.” Thus, in some cases, a subject labeled detector ssDNA includes two signal partners (a signal pair), when one signal partner is a FRET donor moiety and the other signal partner is a FRET acceptor moiety. A subject labeled detector ssDNA that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated (e.g., after cleavage of the RNA molecule by a Type V CRISPR/Cas effector protein (e.g., a Cas12 protein such as Cas12a, Cas12b, Cas12c, Cas12d, Cas12e)).

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table 1. See also: Bajar et al. Sensors (Basel). 2016 Sep. 14; 16(9); and Abraham et al. PLoS One. 2015 Aug. 3; 10(8):e0134436.

TABLE 1
Examples of FRET pairs (donor and acceptor FRET moieties)

| Donor | Acceptor |
|---|---|
| Tryptophan | Dansyl |
| IAEDANS (1) | DDPM (2) |
| BFP | DsRFP |
| Dansyl | Fluorescein isothiocyanate (FITC) |
| Dansyl | Octadecylrhodamine |
| Cyan fluorescent protein (CFP) | Green fluorescent protein (GFP) |
| CF (3) | Texas Red |
| Fluorescein | Tetramethylrhodamine |
| Cy3 | Cy5 |
| GFP | Yellow fluorescent protein (YFP) |
| BODIPY FL (4) | BODIPY FL (4) |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

(1) 5-(2-iodoacetylaminoethyl)aminonaphthalene-1-sulfonic acid
(2) N-(4-dimethylamino-3,5-dinitrophenyl)maleimide
(3) carboxyfluorescein succinimidyl ester
(4) 4,4-difluoro-4-bora-3a,4a-diaza-s-indacene

In some cases, a detectable signal is produced when the labeled detector ssDNA is cleaved (e.g., in some cases, the labeled detector ssDNA comprises a quencher/fluor pair). One signal partner of a signal quenching pair produces a detectable signal and the other signal partner is a quencher moiety that quenches the detectable signal of the first signal partner (i.e., the quencher moiety quenches the signal of the signal moiety such that the signal from the signal moiety is reduced (quenched) when the signal partners are in proximity to one another, e.g., when the signal partners of the signal pair are in close proximity).

For example, in some cases, an amount of detectable signal increases when the labeled detector ssDNA is cleaved. For example, in some cases, the signal exhibited by one signal partner (a signal moiety) is quenched by the other signal partner (a quencher signal moiety), e.g., when both are present on the same ssDNA molecule prior to cleavage by a Type V CRISPR/Cas effector protein (e.g., a Cas12 protein such as Cas12a, Cas12b, Cas12c, Cas12d, Cas12e). Such a signal pair is referred to herein as a “quencher/fluor pair”, “quenching pair”, or “signal quenching pair.” For example, in some cases, one signal partner (e.g., the first signal partner) is a signal moiety that produces a detectable signal that is quenched by the second signal partner (e.g., a quencher moiety). The signal partners of such a quencher/fluor pair will thus produce a detectable signal when the partners are separated (e.g., after cleavage of the detector ssDNA by a Type V CRISPR/Cas effector protein (e.g., a Cas12 protein such as Cas12a, Cas12b, Cas12c, Cas12d, Cas12e)), but the signal will be quenched when the partners are in close proximity (e.g., prior to cleavage of the detector ssDNA by a Type V CRISPR/Cas effector protein (e.g., a Cas12 protein such as Cas12a, Cas12b, Cas12c, Cas12d, Cas12e)).

A quencher moiety can quench a signal from the signal moiety (e.g., prior to cleave of the detector ssDNA by a Type V CRISPR/Cas effector protein (e.g., a Cas12 protein such as Cas12a, Cas12b, Cas12c, Cas12d, Cas12e)) to various degrees. In some cases, a quencher moiety quenches the signal from the signal moiety where the signal detected in the presence of the quencher moiety (when the signal partners are in proximity to one another) is 95% or less of the signal detected in the absence of the quencher moiety (when the signal partners are separated). For example, in some cases, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. In some cases, no signal (e.g., above background) is detected in the presence of the quencher moiety.

In some cases, the signal detected in the absence of the quencher moiety (when the signal partners are separated) is at least 1.2 fold greater (e.g., at least 1.3 fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater) than the signal detected in the presence of the quencher moiety (when the signal partners are in proximity to one another).

In some cases, the signal moiety is a fluorescent label. In some such cases, the quencher moiety quenches the signal (the light signal) from the fluorescent label (e.g., by absorbing energy in the emission spectra of the label). Thus, when the quencher moiety is not in proximity with the signal moiety, the emission (the signal) from the fluorescent label is detectable because the signal is not absorbed by the quencher moiety. Any convenient donor acceptor pair (signal moiety/quencher moiety pair) can be used and many suitable pairs are known in the art.

In some cases the quencher moiety absorbs energy from the signal moiety (also referred to herein as a “detectable label”) and then emits a signal (e.g., light at a different wavelength). Thus, in some cases, the quencher moiety is itself a signal moiety (e.g., a signal moiety can be 6-carboxyfluorescein while the quencher moiety can be 6-carboxy-tetramethylrhodamine), and in some such cases, the pair could also be a FRET pair. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety.

Examples of fluorescent labels include, but are not limited to: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, Pacific Orange, a quantum dot, and a tethered fluorescent protein.

Examples of ATTO dyes include, but are not limited to: ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740.

Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some cases, a quencher moiety is selected from: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

Examples of an ATTO quencher include, but are not limited to: ATTO 540Q, ATTO 580Q, and ATTO 612Q. Examples of a Black Hole Quencher® (BHQ®) include, but are not limited to: BHQ-0 (493 nm), BHQ-1 (534 nm), BHQ-2 (579 nm) and BHQ-3 (672 nm).

For examples of some detectable labels (e.g., fluorescent dyes) and/or quencher moieties, see, e.g., Bao et al., Annu Rev Biomed Eng. 2009; 11:25-47; as well as U.S. Pat. Nos. 8,822,673 and 8,586,718; U.S. patent publications 20140378330, 20140349295, 20140194611, 20130323851, 20130224871, 20110223677, 20110190486, 20110172420, 20060179585 and 20030003486; and international patent applications: WO200142505 and WO200186001, all of which are hereby incorporated by reference in their entirety.

In some cases, cleavage of a labeled detector ssDNA can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA can be detected by a color-shift. Such a shift can be expressed as a loss of an amount of signal of one color (wavelength), a gain in the amount of another color, a change in the ration of one color to another, and the like.

### Type V CRISPR/Cas Effector Proteins

Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbiol. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017 June; 37:67-78: “Diversity, classification and evolution of CRISPR-Cas systems.”

As such in some cases, a subject type V CRISPR/Cas effector protein is a Cas12 protein (e.g., Cas12a, Cas12b, Cas12c). In some cases, a subject type V CRISPR/Cas effector protein is a Cas12 protein such as Cas12a, Cas12b, Cas12c, Cas12d, Cas12e, Cas12d, or Cas12e. In some cases, a subject type V CRISPR/Cas effector protein is a Cas12a protein. In some cases, a subject type V CRISPR/Cas effector protein is a Cas12b protein. In some cases, a subject type V CRISPR/Cas effector protein is a Cas12c protein. In some cases, a subject type V CRISPR/Cas effector protein is a Cas12d protein. In some cases, a subject type V CRISPR/Cas effector protein is a Cas12e protein. In some cases, a subject type V CRISPR/Cas effector protein is protein selected from: Cas12 (e.g., Cas12a, Cas12b, Cas12c, Cas12d, Cas12e), C2c4, C2c8, C2c5, C2c10, and C2c9. In some cases, a subject type V CRISPR/Cas effector protein is protein selected from: C2c4, C2c8, C2c5, C2c10, and C2c9. In some cases, a subject type V CRISPR/Cas effector protein is protein selected from: C2c4, C2c8, and C2c5. In some cases, a subject type V CRISPR/Cas effector protein is protein selected from: C2c10 and C2c9.

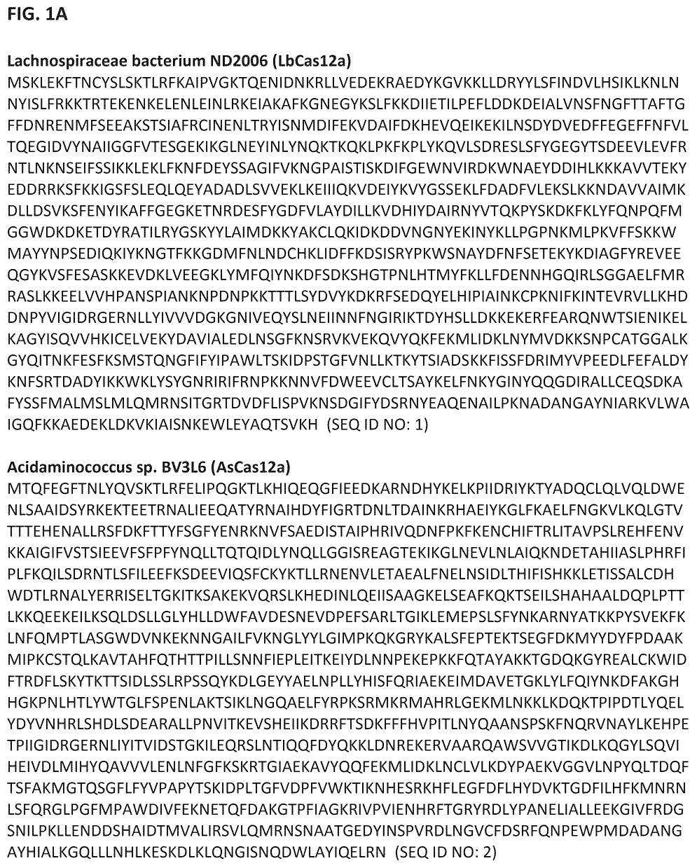

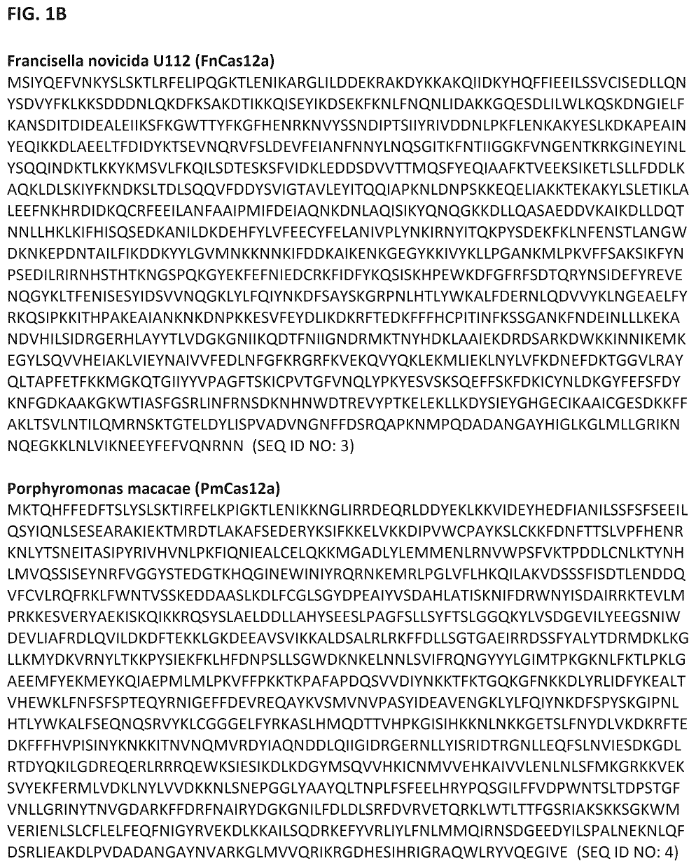

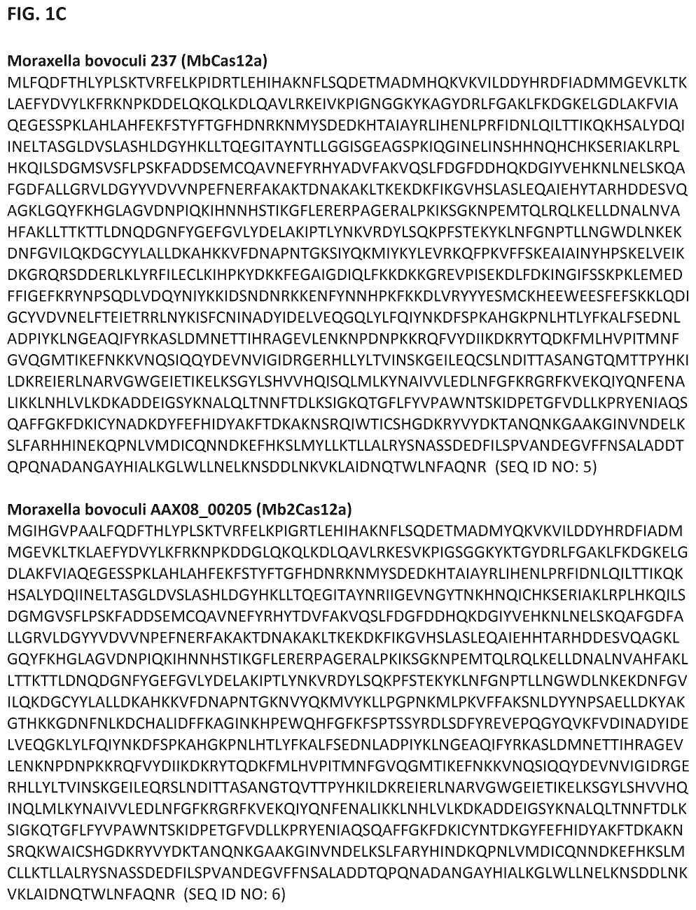

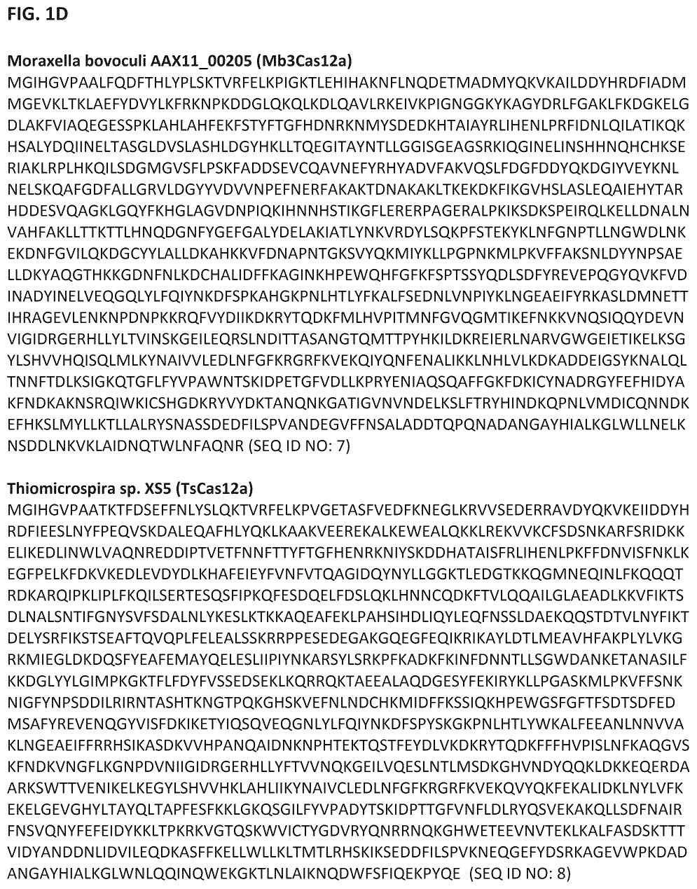

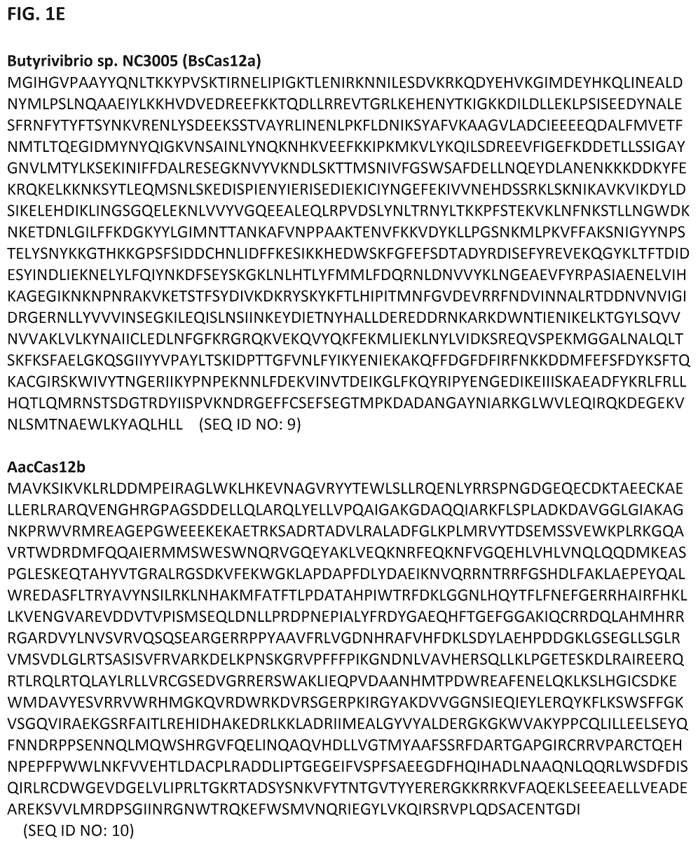

In some cases, the subject type V CRISPR/Cas effector protein is a naturally-occurring protein (e.g., naturally occurs in prokaryotic cells). In other cases, the Type V CRISPR/Cas effector protein is not a naturally-occurring polypeptide (e.g., the effector protein is a variant protein, a chimeric protein, includes a fusion partner, and the like). Examples of naturally occurring Type V CRISPR/Cas effector proteins include, but are not limited to, those depicted in FIGS. 1A-1E. Any Type V CRISPR/Cas effector protein can be suitable for the compositions (e.g., nucleic acids, kits, etc.) and methods of the present disclosure (e.g., as long as the Type V CRISPR/Cas effector protein forms a complex with a guide RNA and exhibits ssDNA cleavage activity of non-target ssDNAs once it is activated (by hybridization of and associated guide RNA to its target DNA).

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein depicted in FIGS. 1A-1E). For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein depicted in FIGS. 1A-1E). In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein depicted in FIGS. 1A-1E). In some cases a type V CRISPR/Cas effector protein comprises an amino acid sequence having 90% or more sequence identity (e.g., 95% or more, 97% or more, 98% or more, 99% or more, or 100% sequence identity) with a Cas12 protein (e.g., Cas12a, Cas12b, Cas12c) (e.g., a Cas12 protein depicted in FIGS. 1A-1E). In some cases a type V CRISPR/Cas effector protein comprises a Cas12 amino acid sequence (e.g., Cas12a, Cas12b, Cas12c) depicted in FIGS. 1A-1E.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12a protein (e.g., a Cas12a protein depicted in FIGS. 1A-1E). For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12a protein (e.g., a Cas12a protein depicted in FIGS. 1A-1E). In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12a protein (e.g., a Cas12a protein depicted in FIGS. 1A-1E). In some cases a type V CRISPR/Cas effector protein comprises an amino acid sequence having 90% or more sequence identity (e.g., 95% or more, 97% or more, 98% or more, 99% or more, or 100% sequence identity) with a Cas12a protein (e.g., a Cas12a protein depicted in FIGS. 1A-1E). In some cases a type V CRISPR/Cas effector protein comprises a Cas12a amino acid sequence depicted in FIGS. 1A-1E.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Lachnospiraceae bacterium ND2006 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Acidaminococcus sp BV3L6 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Francisella novicida U112 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Porphyromonas macacae Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Moraxella bovoculi 237 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Moraxella bovoculi AAX08_00205 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Moraxella bovoculi AAX11_00205 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Thiomicrospira sp. XS5 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Butyrivibrio sp. NC3005 Cas12a protein amino acid sequence depicted in FIGS. 1A-1E. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the AACCas12b amino acid sequence depicted in FIGS. 1A-1E.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12b protein (e.g., a Cas12b protein depicted in FIGS. 1A-1E). For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12b protein (e.g., a Cas12b protein depicted in FIGS. 1A-1E). In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12b protein (e.g., a Cas12b protein depicted in FIGS. 1A-1E). In some cases a type V CRISPR/Cas effector protein comprises an amino acid sequence having 90% or more sequence identity (e.g., 95% or more, 97% or more, 98% or more, 99% or more, or 100% sequence identity) with a Cas12b protein (e.g., a Cas12b protein depicted in FIGS. 1A-1E). In some cases a type V CRISPR/Cas effector protein comprises a Cas12b amino acid sequence depicted in FIGS. 1A-1E.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12, C2c4, C2c8, C2c5, C2c10, or C2c9 protein. For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12, C2c4, C2c8, C2c5, C2c10, or C2c9 protein. In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12, C2c4, C2c8, C2c5, C2c10, or C2c9 protein. In some cases a type V CRISPR/Cas effector protein comprises an amino acid sequence having 90% or more sequence identity (e.g., 95% or more, 97% or more, 98% or more, 99% or more, or 100% sequence identity) with a Cas12, C2c4, C2c8, C2c5, C2c10, or C2c9 protein. In some cases a type V CRISPR/Cas effector protein comprises a Cas12, C2c4, C2c8, C2c5, C2c10, or C2c9 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12, C2c4, C2c8, or C2c5 protein. For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12, C2c4, C2c8, or C2c5 protein. In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12, C2c4, C2c8, or C2c5 protein. In some cases a type V CRISPR/Cas effector protein comprises an amino acid sequence having 90% or more sequence identity (e.g., 95% or more, 97% or more, 98% or more, 99% or more, or 100% sequence identity) with a Cas12, C2c4, C2c8, or C2c5 protein. In some cases a type V CRISPR/Cas effector protein comprises a Cas12, C2c4, C2c8, or C2c5 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a C2c4, C2c8, or C2c5 protein. For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a C2c4, C2c8, or C2c5 protein. In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a C2c4, C2c8, or C2c5 protein. In some cases a type V CRISPR/Cas effector protein comprises an amino acid sequence having 90% or more sequence identity (e.g., 95% or more, 97% or more, 98% or more, 99% or more, or 100% sequence identity) with a C2c4, C2c8, or C2c5 protein. In some cases a type V CRISPR/Cas effector protein comprises a C2c4, C2c8, or C2c5 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12, C2c10, or C2c9 protein. For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12, C2c10, or C2c9 protein. In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12, C2c10, or C2c9 protein. In some cases a type V CRISPR/Cas effector protein comprises an amino acid sequence having 90% or more sequence identity (e.g., 95% or more, 97% or more, 98% or more, 99% or more, or 100% sequence identity) with a Cas12, C2c10, or C2c9 protein. In some cases a type V CRISPR/Cas effector protein comprises a Cas12, C2c10, or C2c9 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a C2c10 or C2c9 protein. For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a C2c10 or C2c9 protein. In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a C2c10 or C2c9 protein. In some cases a type V CRISPR/Cas effector protein comprises an amino acid sequence having 90% or more sequence identity (e.g., 95% or more, 97% or more, 98% or more, 99% or more, or 100% sequence identity) with a C2c10 or C2c9 protein. In some cases a type V CRISPR/Cas effector protein comprises a C2c10 or C2c9 amino acid sequence.

In some cases, a subject type V CRISPR/Cas effector protein (e.g., a Cas12 protein such as Cas12a, Cas12b, Cas12c, Cas12d, Cas12e) is fused to (conjugated to) a heterologous polypeptide. In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some cases, a type V CRISPR/Cas effector protein (e.g., a Cas12 protein) does not include a NLS so that the protein is not targeted to the nucleus (which can be advantageous, e.g., when it desirable to cleave non-target ssDNAs in the cytosol). In some cases, the heterologous polypeptide can provide a tag (i.e., the heterologous polypeptide is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like).

In some cases a type V CRISPR/Cas effector protein (e.g., a Cas12 protein such as Cas12a, Cas12b, Cas12c, Cas12d, Cas12e) includes (is fused to) a nuclear localization signal (NLS) (e.g., in some cases 2 or more, 3 or more, 4 or more, or 5 or more NLSs). Thus, in some cases, a type V CRISPR/Cas effector protein includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases a type V CRISPR/Cas effector protein (e.g., a Cas12 protein such as Cas12a, Cas12b, Cas12c, Cas12d, Cas12e) includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases a type V CRISPR/Cas effector protein includes (is fused to) between 2 and 5 NLSs (e.g., 2-4, or 2-3 NLSs). Non-limiting examples of NLSs include an NLS sequence derived from: the NLS of the SV40 virus large T-antigen, having the amino acid sequence PKKKRKV (SEQ ID NO: 136); the NLS from nucleoplasmin (e.g., the nucleoplasmin bipartite NLS with the sequence KRPAATKKAGQAKKKK (SEQ ID NO: 137)); the c-myc NLS having the amino acid sequence PAAKRVKLD (SEQ ID NO: 138) or RQRRNELKRSP (SEQ ID NO: 139); the hRNPA1 M9 NLS having the sequence NQSSNFGPMKGGNFGGRSSGPYGGGGQYFAKPRNQGGY (SEQ ID NO: 140); the sequence RMRIZFKNKGKDTAELRRRRVEVSVELRKAKKDEQILKRRNV (SEQ ID NO: 141) of the IBB domain from importin-alpha; the sequences VSRKRPRP (SEQ ID NO: 142) and PPKKARED (SEQ ID NO: 143) of the myoma T protein; the sequence PQPKKKPL (SEQ ID NO: 144) of human p53; the sequence SALIKKKKKMAP (SEQ ID NO: 145) of mouse c-abl IV; the sequences DRLRR (SEQ ID NO: 146) and PKQKKRK (SEQ ID NO: 147) of the influenza virus NS1; the sequence RKLKKKIKKL (SEQ ID NO: 148) of the Hepatitis virus delta antigen; the sequence REKKKFLKRR (SEQ ID NO: 149) of the mouse Mx1 protein; the sequence KRKGDEVDGVDEVAKKKSKK (SEQ ID NO: 150) of the human poly(ADP-ribose) polymerase; and the sequence RKCLQAGMNLEARKTKK (SEQ ID NO: 151) of the steroid hormone receptors (human) glucocorticoid. In general, NLS (or multiple NLSs) are of sufficient strength to drive accumulation of the protein in a detectable amount in the nucleus of a eukaryotic cell. Detection of accumulation in the nucleus may be performed by any suitable technique.

### Protospacer Adjacent Motif (PAM)

A Type V CRISPR/Cas effector protein binds to target DNA at a target sequence defined by the region of complementarity between the DNA-targeting RNA and the target DNA. As is the case for many CRISPR/Cas endonucleases, site-specific binding (and/or cleavage) of a double stranded target DNA occurs at locations determined by both (i) base-pairing complementarity between the guide RNA and the target DNA; and (ii) a short motif [referred to as the protospacer adjacent motif (PAM)] in the target DNA.

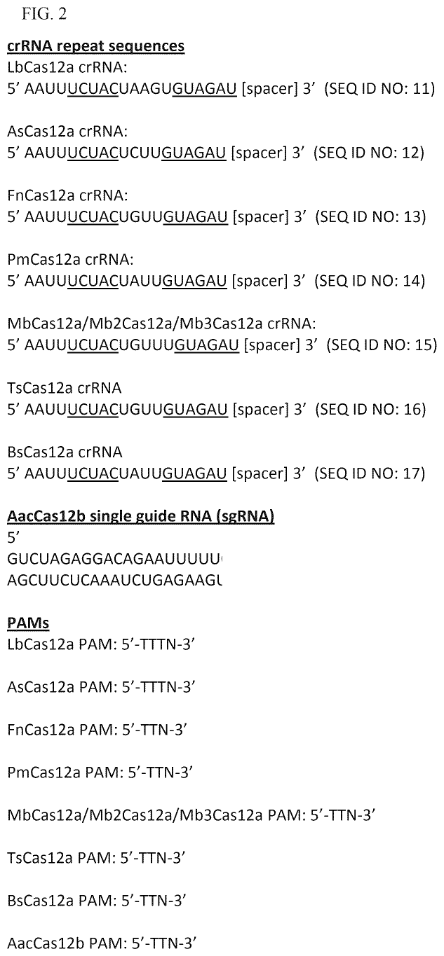

In some cases, the PAM for a Type V CRISPR/Cas effector protein is immediately 5′ of the target sequence (e.g., of the non-complementary strand of the target DNA—the complementary strand hybridizes to the guide sequence of the guide RNA while the non-complementary strand does not directly hybridize with the guide RNA and is the reverse complement of the non-complementary strand). In some cases (e.g., when Cas12a or Cas12b as described herein is used), the PAM sequence is 5′-TTN-3′. In some cases, the PAM sequence is 5′-TTTN-3.′ (e.g., see FIG. 2).

In some cases, different Type V CRISPR/Cas effector proteins (i.e., Type V CRISPR/Cas effector proteins from various species) may be advantageous to use in the various provided methods in order to capitalize on a desired feature (e.g., specific enzymatic characteristics of different Type V CRISPR/Cas effector proteins). Type V CRISPR/Cas effector proteins from different species may require different PAM sequences in the target DNA. Thus, for a particular Type V CRISPR/Cas effector protein of choice, the PAM sequence requirement may be different than the 5′-TTN-3′ or 5′-TTTN-3′ sequence described above. Various methods (including in silico and/or wet lab methods) for identification of the appropriate PAM sequence are known in the art and are routine, and any convenient method can be used.

### Guide RNA

A nucleic acid molecule (e.g., a natural crRNA) that binds to a type V CRISPR/Cas effector protein (e.g., a Cas12 protein such as Cas12a, Cas12b, Cas12c, Cas12d, Cas12e), forming a ribonucleoprotein complex (RNP), and targets the complex to a specific target sequence within a target DNA is referred to herein as a “guide RNA.” It is to be understood that in some cases, a hybrid DNA/RNA can be made such that a guide RNA includes DNA bases in addition to RNA bases—but the term “guide RNA” is still used herein to encompass such hybrid molecules. A subject guide RNA includes a guide sequence (also referred to as a “spacer”)(that hybridizes to target sequence of a target DNA) and a constant region (e.g., a region that is adjacent to the guide sequence and binds to the type V CRISPR/Cas effector protein). A “constant region” can also be referred to herein as a “protein-binding segment.” In some cases, e.g., for Cas12a, the constant region is 5′ of the guide sequence.

### Guide Sequence

The guide sequence has complementarity with (hybridizes to) a target sequence of the target DNA. In some cases, the guide sequence is 15-28 nucleotides (nt) in length (e.g., 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 nt in length). In some cases, the guide sequence is 18-24 nucleotides (nt) in length. In some cases, the guide sequence is at least 15 nt long (e.g., at least 16, 18, 20, or 22 nt long). In some cases, the guide sequence is at least 17 nt long. In some cases, the guide sequence is at least 18 nt long. In some cases, the guide sequence is at least 20 nt long.

In some cases, the guide sequence has 80% or more (e.g., 85% or more, 90% or more, 95% or more, or 100% complementarity) with the target sequence of the target DNA. In some cases, the guide sequence is 100% complementary to the target sequence of the target DNA. In some cases, the target DNA includes at least 15 nucleotides (nt) of complementarity with the guide sequence of the guide RNA.

### Constant Region

Examples of constant regions for guide RNAs that can be used with a type V CRISPR/Cas effector protein (e.g., a Cas12 protein such as Cas12a, Cas12b, Cas12c, Cas12d, Cas12e) are presented in FIG. 2.

In some cases, a subject guide RNA includes a nucleotide sequence having 70% or more identity (e.g., 80% or more, 85% or more, 90% or more, 95% or more, 98% or more, 99% or more, or 100% identity) with any one of the crRNA repeat sequences set forth in FIG. 2. In some cases, a subject guide RNA includes a nucleotide sequence having 90% or more identity (e.g., 95% or more, 98% or more, 99% or more, or 100% identity) with any one of the crRNA repeat sequences set forth in FIG. 2. In some cases, a subject guide RNA includes a crRNA nucleotide sequence set forth in FIG. 2.

In some cases, the guide RNA includes a double stranded RNA duplex (dsRNA duplex). In some cases, a guide RNA includes a dsRNA duplex with a length of from 2 to 12 bp (e.g., from 2 to 10 bp, 2 to 8 bp, 2 to 6 bp, 2 to 5 bp, 2 to 4 bp, 3 to 12 bp, 3 to 10 bp, 3 to 8 bp, 3 to 6 bp, 3 to 5 bp, 3 to 4 bp, 4 to 12 bp, 4 to 10 bp, 4 to 8 bp, 4 to 6 bp, or 4 to 5 bp). In some cases, a guide RNA includes a dsRNA duplex that is 2 or more bp in length (e.g., 3 or more, 4 or more, 5 or more, 6 or more, or 7 or more bp in length). In some cases, a guide RNA includes a dsRNA duplex that is longer than the dsRNA duplex of a corresponding wild type guide RNA. In some cases, a guide RNA includes a dsRNA duplex that is shorter than the dsRNA duplex of a corresponding wild type guide RNA.

In some cases, the constant region of a guide RNA is 15 or more nucleotides (nt) in length (e.g., 18 or more, 20 or more, 21 or more, 22 or more, 23 or more, 24 or more, 25 or more, 26 or more, 27 or more, 28 or more, 29 or more, 30 or more, 31 or more nt, 32 or more, 33 or more, 34 or more, or 35 or more nt in length). In some cases, the constant region of a guide RNA is 18 or more nt in length.

In some cases, the constant region of a guide RNA has a length in a range of from 12 to 100 nt (e.g., from 12 to 90, 12 to 80, 12 to 70, 12 to 60, 12 to 50, 12 to 40, 15 to 100, 15 to 90, 15 to 80, 15 to 70, 15 to 60, 15 to 50, 15 to 40, 20 to 100, 20 to 90, 20 to 80, 20 to 70, 20 to 60, 20 to 50, 20 to 40, 25 to 100, 25 to 90, 25 to 80, 25 to 70, 25 to 60, 25 to 50, 25 to 40, 28 to 100, 28 to 90, 28 to 80, 28 to 70, 28 to 60, 28 to 50, 28 to 40, 29 to 100, 29 to 90, 29 to 80, 29 to 70, 29 to 60, 29 to 50, or 29 to 40 nt). In some cases, the constant region of a guide RNA has a length in a range of from 28 to 100 nt. In some cases, the region of a guide RNA that is 5′ of the guide sequence has a length in a range of from 28 to 40 nt.

In some cases, the constant region of a guide RNA is truncated relative to (shorter than) the corresponding region of a corresponding wild type guide RNA. In some cases, the constant region of a guide RNA is extended relative to (longer than) the corresponding region of a corresponding wild type guide RNA. In some cases, a subject guide RNA is 30 or more nucleotides (nt) in length (e.g., 34 or more, 40 or more, 45 or more, 50 or more, 55 or more, 60 or more, 65 or more, 70 or more, or 80 or more nt in length). In some cases, the guide RNA is 35 or more nt in length.

### Precursor Guide RNA Array

A Type V CRISPR/Cas effector protein (e.g., a Cas12 protein such as Cas12a, Cas12b, Cas12c, Cas12d, Cas12e) can cleave a precursor guide RNA into a mature guide RNA, e.g., by endoribonucleolytic cleavage of the precursor. A Type V CRISPR/Cas effector protein (e.g., a Cas12 protein such as Cas12a, Cas12b, Cas12c, Cas12d, Cas12e) can cleave a precursor guide RNA array (that includes more than one guide RNA arrayed in tandem) into two or more individual guide RNAs. Thus, in some cases a precursor guide RNA array comprises two or more (e.g., 3 or more, 4 or more, 5 or more, 2, 3, 4, or 5) guide RNAs (e.g., arrayed in tandem as precursor molecules). In other words, in some cases, two or more guide RNAs can be present on an array (a precursor guide RNA array). A Type V CRISPR/Cas effector protein (e.g., a Cas12 protein such as Cas12a, Cas12b, Cas12c, Cas12d, Cas12e) can cleave the precursor guide RNA array into individual guide RNAs

In some cases a subject guide RNA array includes 2 or more guide RNAs (e.g., 3 or more, 4 or more, 5 or more, 6 or more, or 7 or more, guide RNAs). The guide RNAs of a given array can target (i.e., can include guide sequences that hybridize to) different target sites of the same target DNA (e.g., which can increase sensitivity of detection) and/or can target different target DNA molecules (e.g., single nucleotide polymorphisms (SNPs), different strains of a particular virus, etc.), and such could be used for example to detect multiple strains of a virus. In some cases, each guide RNA of a precursor guide RNA array has a different guide sequence. In some cases, two or more guide RNAs of a precursor guide RNA array have the same guide sequence.

In some cases, the precursor guide RNA array comprises two or more guide RNAs that target different target sites within the same target DNA molecule. For example, such a scenario can in some cases increase sensitivity of detection by activating Type V CRISPR/Cas effector protein (e.g., a Cas12 protein such as Cas12a, Cas12b, Cas12c, Cas12d, Cas12e) when either one hybridizes to the target DNA molecule. As such, in some cases as subject composition (e.g., kit) or method includes two or more guide RNAs (in the context of a precursor guide RNA array, or not in the context of a precursor guide RNA array, e.g., the guide RNAs can be mature guide RNAs).

In some cases, the precursor guide RNA array comprises two or more guide RNAs that target different target DNA molecules. For example, such a scenario can result in a positive signal when any one of a family of potential target DNAs is present. Such an array could be used for targeting a family of transcripts, e.g., based on variation such as single nucleotide polymorphisms (SNPs) (e.g., for diagnostic purposes). Such could also be useful for detecting whether any one of a number of different strains of virus is present. Such could also be useful for detecting whether any one of a number of different species, strains, isolates, or variants of a bacterium is present (e.g., different species, strains, isolates, or variants of Mycobacterium, different species, strains, isolates, or variants of Neisseria, different species, strains, isolates, or variants of Staphylococcus aureus; different species, strains, isolates, or variants of E. coli; etc.). As such, in some cases as subject composition (e.g., kit) or method includes two or more guide RNAs (in the context of a precursor guide RNA array, or not in the context of a precursor guide RNA array, e.g., the guide RNAs can be mature guide RNAs).

### Nucleic Acid Modifications

In some cases, a labeled detector ssDNA (and/or a guide RNA) comprises one or more modifications, e.g., a base modification, a backbone modification, a sugar modification, etc., to provide the nucleic acid with a new or enhanced feature (e.g., improved stability). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Modified Backbones and Modified Internucleoside Linkages

Examples of suitable modifications include modified nucleic acid backbones and non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a labeled detector ssDNA (and/or a guide RNA) comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some cases, a labeled detector ssDNA (and/or a guide RNA) comprises a 6-membered morpholino ring in place of a ribose ring. In some cases, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A labeled detector ssDNA (and/or a guide RNA) can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Modified Sugar Moieties

A labeled detector ssDNA (and/or a guide RNA) can also include one or more substituted sugar moieties. Suitable polynucleotides comprise a sugar substituent group selected from: OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A labeled detector ssDNA (and/or a guide RNA) may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b)(1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

Introducing Components into a Target Cell

A guide RNA (or a nucleic acid comprising a nucleotide sequence encoding same) and/or a type V CRISPR/Cas effector protein can be introduced into a host cell by any of a variety of well-known methods. As a non-limiting example, a guide RNA and/or type V CRISPR/Cas effector protein can be combined with a lipid. As another non-limiting example, a guide RNA and/or type V CRISPR/Cas effector protein can be combined with a particle, or formulated into a particle.

Methods of introducing a nucleic acid and/or protein into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like.

A guide RNA can be introduced, e.g., as a DNA molecule encoding the guide RNA, or can be provided directly as an RNA molecule (or a hybrid molecule when applicable). In some cases, a type V CRISPR/Cas effector protein is provided as a nucleic acid (e.g., an mRNA, a DNA, a plasmid, an expression vector, a viral vector, etc.) that encodes the protein. In some cases, the type V CRISPR/Cas effector protein is provided directly as a protein (e.g., without an associated guide RNA or with an associate guide RNA, i.e., as a ribonucleoprotein complex—RNP). Like a guide RNA, a type V CRISPR/Cas effector protein can be introduced into a cell (provided to the cell) by any convenient method; such methods are known to those of ordinary skill in the art. As an illustrative example, a type V CRISPR/Cas effector protein can be injected directly into a cell (e.g., with or without a guide RNA or nucleic acid encoding a guide RNA). As another example, a preformed complex of a type V CRISPR/Cas effector protein and a guide RNA (an RNP) can be introduced into a cell (e.g., eukaryotic cell) (e.g., via injection, via nucleofection; via a protein transduction domain (PTD) conjugated to one or more components, e.g., conjugated to the type V CRISPR/Cas effector protein, conjugated to a guide RNA; etc.).

In some cases, a nucleic acid (e.g., a guide RNA; a nucleic acid comprising a nucleotide sequence encoding a type V CRISPR/Cas effector protein; etc.) and/or a polypeptide (e.g., a type V CRISPR/Cas effector protein) is delivered to a cell (e.g., a target host cell) in a particle, or associated with a particle. The terms “particle” and “nanoparticle” can be used interchangeably, as appropriate.

This can be achieved, e.g., using particles or lipid envelopes, e.g., a ribonucleoprotein (RNP) complex can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5).

A type V CRISPR/Cas effector protein (or an mRNA comprising a nucleotide sequence encoding the protein) and/or guide RNA (or a nucleic acid such as one or more expression vectors encoding the guide RNA) may be delivered simultaneously using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used. Doses of about 5 mg/kg can be used, with single or multiple doses, depending on various factors, e.g., the target tissue.

Lipidoid compounds (e.g., as described in US patent publication 20110293703) are also useful in the administration of polynucleotides, and can be used. In one aspect, aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used, sugar-based particles may be used, for example GalNAc, as described with reference to WO2014118272 (incorporated herein by reference) and Nair, J K et al., 2014, Journal of the American Chemical Society 136 (49), 16958-16961). In some cases, lipid nanoparticles (LNPs) are used. Spherical Nucleic Acid (SNA™) constructs and other nanoparticles (particularly gold nanoparticles) can be used to a target cell. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192. Semi-solid and soft nanoparticles are also suitable for delivery. An exosome can be used for delivery. Exosomes are endogenous nano-vesicles that transport RNAs and proteins, and which can deliver RNA to the brain and other target organs. Supercharged proteins can be used for delivery to a cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo. Cell Penetrating Peptides (CPPs) can be used for delivery. CPPs typically have an amino acid composition that either contains a high relative abundance of positively charged amino acids such as lysine or arginine or has sequences that contain an alternating pattern of polar/charged amino acids and non-polar, hydrophobic amino acids.

### Target Cells of Interest

Suitable target cells (which can comprise target nucleic acids such as genomic DNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

Because the guide RNA provides specificity by hybridizing to target nucleic acid, a mitotic and/or post-mitotic cell of interest in the disclosed methods may include a cell of any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell of an invertebrate animal (e.g. fruit fly, cnidarian, echinoderm, nematode, etc.), a cell of a vertebrate animal (e.g., fish, amphibian, reptile, bird, mammal), a cell of a mammal, a cell of a rodent, a cell of a human, etc.).

Plant cells include cells of a monocotyledon, and cells of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

### Kits

The present disclosure provides a kit for detecting a target DNA, e.g., in a sample comprising a plurality of DNAs. In some cases, the kit comprises: (a) a labeled detector ssDNA (e.g., a labeled detector ssDNA comprising a fluorescence-emitting dye pair, i.e., a FRET pair and/or a quencher/fluor pair); and (b) one or more of: (i) a guide RNA, and/or a nucleic acid encoding said guide RNA; (ii); a precursor guide RNA array comprising two or more guide RNAs (e.g., each of which has a different guide sequence), and/or a nucleic acid encoding the precursor guide RNA array; and (iii) a Type V CRISPR/Cas effector protein (e.g., a Cas12 protein such as Cas12a, Cas12b, Cas12c, Cas12d, Cas12e), and/or a nucleic acid encoding said Type V CRISPR/Cas effector protein. In some cases a nucleic acid encoding a precursor guide RNA array includes sequence insertion sites for the insertion of guide sequences by a user.

In some cases, a subject kit comprises: (a) a labeled detector ssDNA comprising a fluorescence-emitting dye pair, i.e., a FRET pair and/or a quencher/fluor pair; and (b) one or more of: (i) a guide RNA, and/or a nucleic acid encoding said guide RNA; (ii); a precursor guide RNA array comprising two or more guide RNAs (e.g., each of which has a different guide sequence), and/or a nucleic acid encoding the precursor guide RNA array; and (iii) a Type V CRISPR/Cas effector protein (e.g., a Cas12 protein such as Cas12a, Cas12b, Cas12c, Cas12d, Cas12e), and/or a nucleic acid encoding said Type V CRISPR/Cas effector protein.

### Positive Controls

A kit of the present disclosure (e.g., one that comprises a labeled detector ssDNA and a type V CRISPR/Cas effector protein) can also include a positive control target DNA. In some cases, the kit also includes a positive control guide RNA that comprises a nucleotide sequence that hybridizes to the control target DNA. In some cases, the positive control target DNA is provided in various amounts, in separate containers. In some cases, the positive control target DNA is provided in various known concentrations, in separate containers, along with control non-target DNAs.

### Nucleic Acids

While the RNAs of the disclosure (e.g., guide RNAs and precursor guide RNA arrays) can be synthesized using any convenient method (e.g., chemical synthesis, in vitro using an RNA polymerase enzyme, e.g., T7 polymerase, T3 polymerase, SP6 polymerase, etc.), nucleic acids encoding guide RNAs and/or precursor guide RNA arrays are also envisioned. Additionally, while Type V CRISPR/Cas effector proteins (e.g., a Cas12 protein such as Cas12a, Cas12b, Cas12c, Cas12d, Cas12e) of the disclosure can be provided (e.g., as part of a kit) in protein form, nucleic acids (such as mRNA and/or DNA) encoding the Type V CRISPR/Cas effector protein (e.g., a Cas12 protein such as Cas12a, Cas12b, Cas12c, Cas12d, Cas12e)(s) can also be provided.

For example, in some cases, a kit of the present disclosure comprises a nucleic acid (e.g., a DNA, e.g., a recombinant expression vector) that comprises a nucleotide sequence encoding a guide RNA. In some cases, the nucleotide sequence encodes a guide RNA without a guide sequence. For example, in some cases, the nucleic acid comprises a nucleotide sequence encoding a constant region of a guide RNA (a guide RNA without a guide sequence), and comprises an insertion site for a nucleic acid encoding a guide sequence. In some cases, a kit of the present disclosure comprises a nucleic acid (e.g., an mRNA, a DNA, e.g., a recombinant expression vector) that comprises a nucleotide sequence encoding a Type V CRISPR/Cas effector protein (e.g., a Cas12 protein such as Cas12a, Cas12b, Cas12c, Cas12d, Cas12e).

In some cases, a kit of the present disclosure comprises a nucleic acid (e.g., a DNA, e.g., a recombinant expression vector) that comprises a nucleotide sequence encoding a precursor guide RNA array (e.g., in some cases where each guide RNA of the array has a different guide sequence). In some cases, one or more of the encoded guide RNAs of the array does not have a guide sequence, e.g., the nucleic acid can include insertion site(s) for the guide sequence(s) of one or more of the guide RNAs of the array. In some cases, a subject guide RNA can include a handle from a precursor crRNA but does not necessarily have to include multiple guide sequences.

In some cases, the guide RNA-encoding nucleotide sequence (and/or the precursor guide RNA array-encoding nucleotide sequence) is operably linked to a promoter, e.g., a promoter that is functional in a prokaryotic cell, a promoter that is functional in a eukaryotic cell, a promoter that is functional in a mammalian cell, a promoter that is functional in a human cell, and the like. In some cases, a nucleotide sequence encoding a Type V CRISPR/Cas effector protein (e.g., a Cas12 protein such as Cas12a, Cas12b, Cas12c, Cas12d, Cas12e) is operably linked to a promoter, e.g., a promoter that is functional in a prokaryotic cell, a promoter that is functional in a eukaryotic cell, a promoter that is functional in a mammalian cell, a promoter that is functional in a human cell, a cell type-specific promoter, a regulatable promoter, a tissue-specific promoter, and the like.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-45 (SET A) and 1-54 (SET B) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### Set A

1. A method of detecting a target DNA in a sample, the method comprising:
(a) contacting the sample with:
   (i) a type V CRISPR/Cas effector protein;
   (ii) a guide RNA comprising: a region that binds to the type V CRISPR/Cas effector protein, and a guide sequence that hybridizes with the target DNA; and
   (iii) a detector DNA that is single stranded and does not hybridize with the guide sequence of the guide RNA; and
(b) measuring a detectable signal produced by cleavage of the single stranded detector DNA by the type V CRISPR/Cas effector protein, thereby detecting the target DNA.

2. The method of 1, comprising contacting the sample with a precursor guide RNA array, wherein the type V CRISPR/Cas effector protein cleaves the precursor guide RNA array to produce said guide RNA and at least one additional guide RNA.

3. The method of 1 or 2, wherein the target DNA is single stranded.

4. The method of 1 or 2, wherein the target DNA is double stranded.

5. The method of any one of 1-4, wherein the target DNA is viral DNA.

6. The method of any one of 1-4, wherein the target DNA is papovavirus, hepdnavirus, herpesvirus, adenovirus, poxvirus, or parvovirus DNA.

7. The method of any one of 1-4, wherein the type V CRISPR/Cas effector protein is a Cas12 protein.

8. The method of any one of 1-6, wherein the type V CRISPR/Cas effector protein is a Cas12a (Cpf1) or Cas12b (C2c1) protein.

9. The method according to any one of 1-8, wherein the sample comprises DNA molecules from a cell lysate.

10. The method according to any one of 1-9, wherein the sample comprises cells.

11. The method according to any one of 1-10, wherein said contacting is carried out inside of a cell in vitro, ex vivo, or in vivo.

12. The method according to 11, wherein the cell is a eukaryotic cell.

13. The method according to any one of 1-12, wherein the target DNA can be detected at a concentration as low as 200 fM.

14. The method according to any one of 1-13, comprising determining an amount of the target DNA present in the sample.

15. The method according to 14, wherein said determining comprises:
measuring the detectable signal to generate a test measurement;
measuring a detectable signal produced by a reference sample or cell to generate a reference measurement; and
comparing the test measurement to the reference measurement to determine an amount of target DNA present in the sample.

16. The method according to any one of 1-15, wherein measuring a detectable signal comprises one or more of: gold nanoparticle based detection, fluorescence polarization, colloid phase transition/dispersion, electrochemical detection, and semiconductor-based sensing.

17. The method according to any one of 1-16, wherein the single stranded detector DNA comprises a fluorescence-emitting dye pair.

18. The method according to 17, wherein the fluorescence-emitting dye pair produces an amount of detectable signal prior to cleavage of the single stranded detector DNA, and the amount of detectable signal is reduced after cleavage of the single stranded detector DNA.

19. The method according to 17, wherein the single stranded detector DNA produces a first detectable signal prior to being cleaved and a second detectable signal after cleavage of the single stranded detector DNA.

20. The method according to any one of 17-19, wherein the fluorescence-emitting dye pair is a fluorescence resonance energy transfer (FRET) pair.

21. The method according to 17, wherein an amount of detectable signal increases after cleavage of the single stranded detector DNA.

22. The method according to 17 or 21, wherein the fluorescence-emitting dye pair is a quencher/fluor pair.

23. The method according to any one of 17-22, wherein the single stranded detector DNA comprises two or more fluorescence-emitting dye pairs.

24. The method according to 23, wherein said two or more fluorescence-emitting dye pairs include a fluorescence resonance energy transfer (FRET) pair and a quencher/fluor pair.

25. The method according to any one of 1-24, wherein the single stranded detector DNA comprises a modified nucleobase, a modified sugar moiety, and/or a modified nucleic acid linkage.

26. A kit for detecting a target DNA in a sample, the kit comprising:
(a) a guide RNA, or a nucleic acid encoding the guide RNA, or a precursor guide RNA array comprising the guide RNA, or a nucleic acid encoding the precursor guide RNA array; wherein the guide RNA comprises: a region that binds to a type V CRISPR/Cas effector protein, and a guide sequence that is complementary to a target DNA; and
(b) a labeled detector DNA that is single stranded and does not hybridize with the guide sequence of the guide RNA.

27. The kit of 26, further comprising a type V CRISPR/Cas effector protein.

28. The kit of 27, wherein the type V CRISPR/Cas effector protein is a Cas12 protein.

29. The kit of 27, wherein the type V CRISPR/Cas effector protein is a Cas12a (Cpf1) or Cas12b (C2c1) protein.

30. The kit of any one of 26-29, wherein the single stranded detector DNA comprises a fluorescence-emitting dye pair.

31. The kit of 30, wherein the fluorescence-emitting dye pair is a FRET pair.

32. The kit of 30, wherein the fluorescence-emitting dye pair is a quencher/fluor pair.

33. The kit of any one of 30-32, wherein the single stranded detector DNA comprises two or more fluorescence-emitting dye pairs.

34. The kit of 33, wherein said two or more fluorescence-emitting dye pairs include a first fluorescence-emitting dye pair that produces a first detectable signal and a second fluorescence-emitting dye pair that produces a second detectable signal.

35. A method of cleaving single stranded DNAs (ssDNAs), the method comprising:
contacting a population of nucleic acids, wherein said population comprises a target DNA and a plurality of non-target ssDNAs, with:
(i) a type V CRISPR/Cas effector protein; and
(ii) a guide RNA comprising: a region that binds to the type V CRISPR/Cas effector protein, and a guide sequence that hybridizes with the target DNA,
wherein the type V CRISPR/Cas effector protein cleaves non-target ssDNAs of said plurality.

36. The method of 35, comprising contacting the sample with a precursor guide RNA array, wherein the type V CRISPR/Cas effector protein cleaves the precursor guide RNA array to produce said guide RNA and at least one additional guide RNA.

37. The method of 35 or 36, wherein said contacting is inside of a cell in vitro, ex vivo, or in vivo.

38. The method of 37, wherein the cell is a eukaryotic cell.

39. The method of 38, wherein the eukaryotic cell is a plant cell.

40. The method of any one of 37-39, wherein the non-target ssDNAs are foreign to the cell.

41. The method of 40, wherein the non-target ssDNAs are viral DNAs.

42. The method of any one of 35-41, wherein the target DNA is single stranded.

43. The method of any one of 35-41, wherein the target DNA is double stranded.

44. The method of any one of 35-43, wherein the target DNA is viral DNA.

45. The method of any one of 35-43, wherein the target DNA is papovavirus, hepdnavirus, herpesvirus, adenovirus, poxvirus, or parvovirus DNA.

### Set B

1. A method of detecting a target DNA in a sample, the method comprising:
(a) contacting the sample with:
   (i) a type V CRISPR/Cas effector protein;
   (ii) a guide RNA comprising: a region that binds to the type V CRISPR/Cas effector protein, and a guide sequence that hybridizes with the target DNA; and
   (iii) a detector DNA that is single stranded and does not hybridize with the guide sequence of the guide RNA; and
(b) measuring a detectable signal produced by cleavage of the single stranded detector DNA by the type V CRISPR/Cas effector protein, thereby detecting the target DNA.

2. The method of 1, comprising contacting the sample with a precursor guide RNA array, wherein the type V CRISPR/Cas effector protein cleaves the precursor guide RNA array to produce said guide RNA and at least one additional guide RNA.

3. The method of 1 or 2, wherein the target DNA is single stranded.

4. The method of 1 or 2, wherein the target DNA is double stranded.

5. The method of any one of 1-4, wherein the target DNA is viral DNA.

6. The method of any one of 1-4, wherein the target DNA is papovavirus, hepdnavirus, herpesvirus, adenovirus, poxvirus, or parvovirus DNA.

7. The method of any one of 1-4, wherein the type V CRISPR/Cas effector protein is a Cas12 protein.

8. The method of any one of 1-6, wherein the type V CRISPR/Cas effector protein is a Cas12a (Cpf1) or Cas12b (C2c1) protein.

9. The method of any one of 1-6, wherein the type V CRISPR/Cas effector protein is a Cas12d (CasY) or Cas12e (CasX) protein.

10. The method according to any one of 1-9, wherein the sample comprises DNA molecules from a cell lysate.

11. The method according to any one of 1-10, wherein the sample comprises cells.

12. The method according to any one of 1-11, wherein said contacting is carried out inside of a cell in vitro, ex vivo, or in vivo.

13. The method according to 12, wherein the cell is a eukaryotic cell.

14. The method according to any one of 1-13, wherein the target DNA can be detected at a concentration as low as 200 fM.

15. The method according to any one of 1-14, comprising determining an amount of the target DNA present in the sample.

16. The method according to 15, wherein said determining comprises:
measuring the detectable signal to generate a test measurement;
measuring a detectable signal produced by a reference sample or cell to generate a reference measurement; and
comparing the test measurement to the reference measurement to determine an amount of target DNA present in the sample.

17. The method according to any one of 1-16, wherein measuring a detectable signal comprises one or more of: gold nanoparticle based detection, fluorescence polarization, colloid phase transition/dispersion, electrochemical detection, and semiconductor-based sensing.

18. The method according to any one of 1-17, wherein the single stranded detector DNA comprises a fluorescence-emitting dye pair.

19. The method according to 18, wherein the fluorescence-emitting dye pair produces an amount of detectable signal prior to cleavage of the single stranded detector DNA, and the amount of detectable signal is reduced after cleavage of the single stranded detector DNA.

20. The method according to 18, wherein the single stranded detector DNA produces a first detectable signal prior to being cleaved and a second detectable signal after cleavage of the single stranded detector DNA.

21. The method according to any one of 18-20, wherein the fluorescence-emitting dye pair is a fluorescence resonance energy transfer (FRET) pair.

22. The method according to 18, wherein an amount of detectable signal increases after cleavage of the single stranded detector DNA.

23. The method according to 18 or 22, wherein the fluorescence-emitting dye pair is a quencher/fluor pair.

24. The method according to any one of 18-23, wherein the single stranded detector DNA comprises two or more fluorescence-emitting dye pairs.

25. The method according to 24, wherein said two or more fluorescence-emitting dye pairs include a fluorescence resonance energy transfer (FRET) pair and a quencher/fluor pair.

26. The method according to any one of 1-25, wherein the single stranded detector DNA comprises a modified nucleobase, a modified sugar moiety, and/or a modified nucleic acid linkage.

27. The method according to any one of 1-26, wherein the method comprises amplifying nucleic acids in the sample.

28. The method according to 27, wherein said amplifying comprises isothermal amplification.

29. The method according to 28, wherein the isothermal amplification comprises recombinase polymerase amplification (RPA).

30. The method according to any one of 27-29, wherein said amplifying begins prior to the contacting of step (a).

31. The method according to any one of 27-29, wherein said amplifying begins together with the contacting of step (a).

32. A kit for detecting a target DNA in a sample, the kit comprising:
(a) a guide RNA, or a nucleic acid encoding the guide RNA, or a precursor guide RNA array comprising the guide RNA, or a nucleic acid encoding the precursor guide RNA array; wherein the guide RNA comprises: a region that binds to a type V CRISPR/Cas effector protein, and a guide sequence that is complementary to a target DNA; and
(b) a labeled detector DNA that is single stranded and does not hybridize with the guide sequence of the guide RNA.

33. The kit of 32, further comprising a type V CRISPR/Cas effector protein.

34. The kit of 33, wherein the type V CRISPR/Cas effector protein is a Cas12 protein.

35. The kit of 33, wherein the type V CRISPR/Cas effector protein is a Cas12a (Cpf1) or Cas12b (C2c1) protein.

36. The kit of 33, wherein the type V CRISPR/Cas effector protein is a Cas12d (CasY) or Cas12e (CasX) protein.

37. The kit of any one of 32-36, wherein the single stranded detector DNA comprises a fluorescence-emitting dye pair.

38. The kit of 37, wherein the fluorescence-emitting dye pair is a FRET pair.

39. The kit of 37, wherein the fluorescence-emitting dye pair is a quencher/fluor pair.

40. The kit of any one of 37-39, wherein the single stranded detector DNA comprises two or more fluorescence-emitting dye pairs.

41. The kit of 40, wherein said two or more fluorescence-emitting dye pairs include a first fluorescence-emitting dye pair that produces a first detectable signal and a second fluorescence-emitting dye pair that produces a second detectable signal.

42. The kit of any one of 32-41, further comprising nucleic acid amplification components.

43. The kit of 42, wherein the nucleic acid amplification components are components for recombinase polymerase amplification (RPA).

44. A method of cleaving single stranded DNAs (ssDNAs), the method comprising:
contacting a population of nucleic acids, wherein said population comprises a target DNA and a plurality of non-target ssDNAs, with:
(i) a type V CRISPR/Cas effector protein; and
(ii) a guide RNA comprising: a region that binds to the type V CRISPR/Cas effector protein, and a guide sequence that hybridizes with the target DNA,
wherein the type V CRISPR/Cas effector protein cleaves non-target ssDNAs of said plurality.

45. The method of 44, comprising contacting the sample with a precursor guide RNA array, wherein the type V CRISPR/Cas effector protein cleaves the precursor guide RNA array to produce said guide RNA and at least one additional guide RNA.

46. The method of 44 or 45, wherein said contacting is inside of a cell in vitro, ex vivo, or in vivo.

47. The method of 46, wherein the cell is a eukaryotic cell.

48. The method of 47, wherein the eukaryotic cell is a plant cell.

49. The method of any one of 46-48, wherein the non-target ssDNAs are foreign to the cell.

50. The method of 49, wherein the non-target ssDNAs are viral DNAs.

51. The method of any one of 44-50, wherein the target DNA is single stranded.

52. The method of any one of 44-50, wherein the target DNA is double stranded.

53. The method of any one of 44-52, wherein the target DNA is viral DNA.

54. The method of any one of 44-52, wherein the target DNA is papovavirus, hepdnavirus, herpesvirus, adenovirus, poxvirus, or parvovirus DNA.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

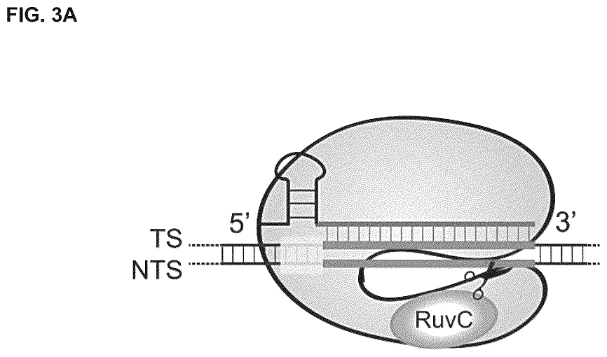

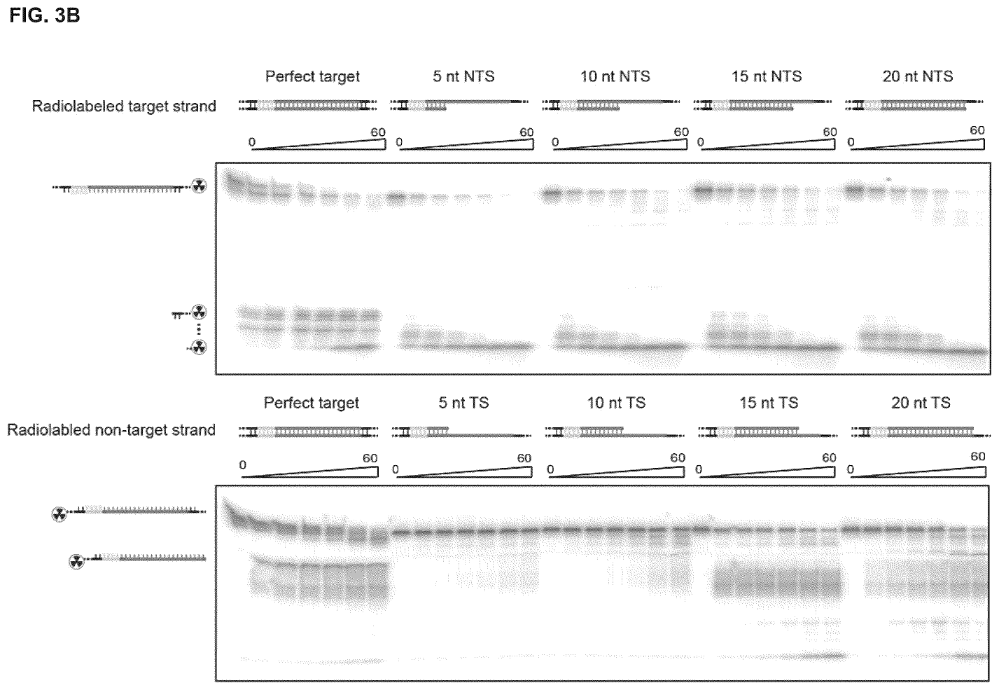

FIGS. 3A-3B. Non-complementary strand cleavage is dictated by complementary strand recognition. The length of the non-target strand (NTS) (top gel) or target strand (TS) (bottom gel) was altered to determine the substrate requirements for Cas12 cleavage. LbCas12a-crRNA complexes are in large excess over radiolabeled substrates, and cleavage products are resolved by denaturing polyacrylamide gel electrophoresis (PAGE). The TS was trimmed to single nucleotides regardless of the length of the NTS, whereas the NTS was cleaved only when at least 15 nt of complementary TS is present.

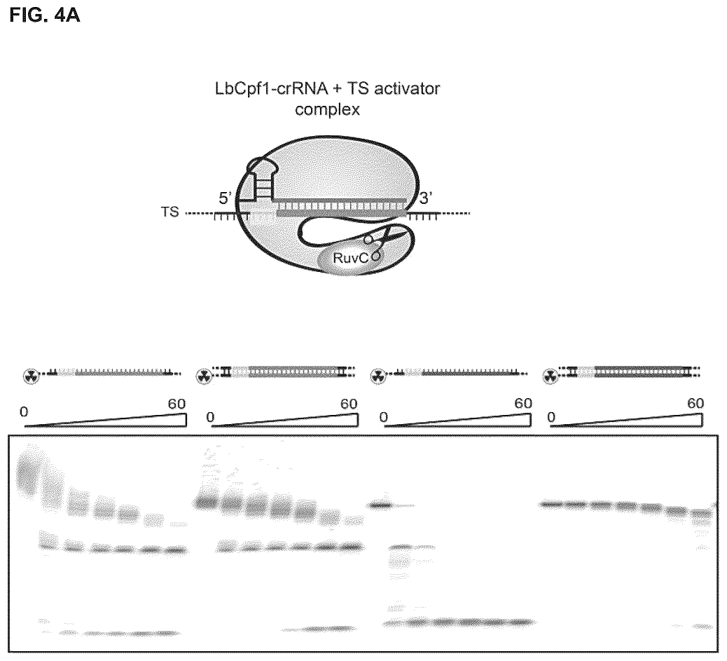

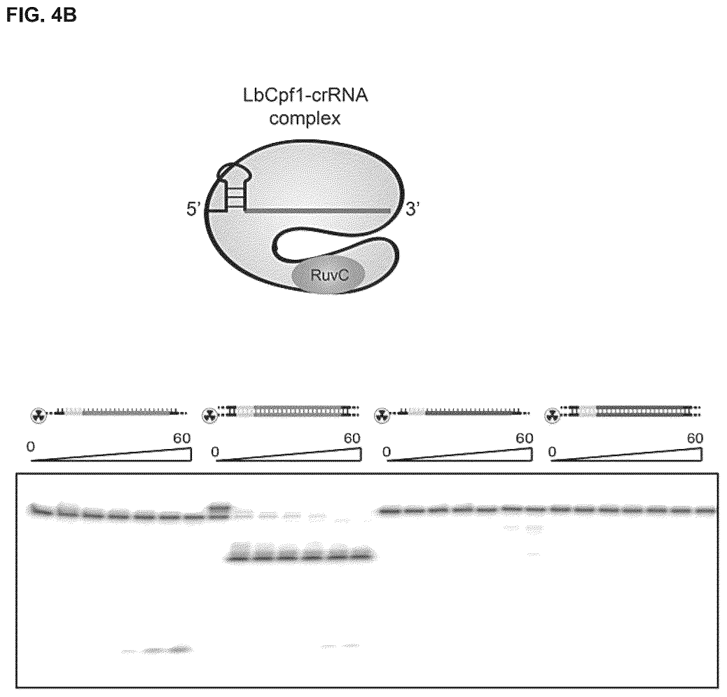

FIGS. 4A-4B. Complementary strand binding unleashes non-specific DNase activity by Cas12a. It was tested whether a non-complementary, random ssDNA is prone to degradation upon Cas12a activation by a complementary target strand. LbCas12a-crRNA complexes are in large excess over radiolabeled substrates, and cleavage products are resolved by denaturing PAGE. The random ssDNA radiolabeled target (blue) was degraded only when LbCas12a was pre-complexed with an “activator” complementary target strand. The random dsDNA radiolabeled target (blue) was protected from cleavage.

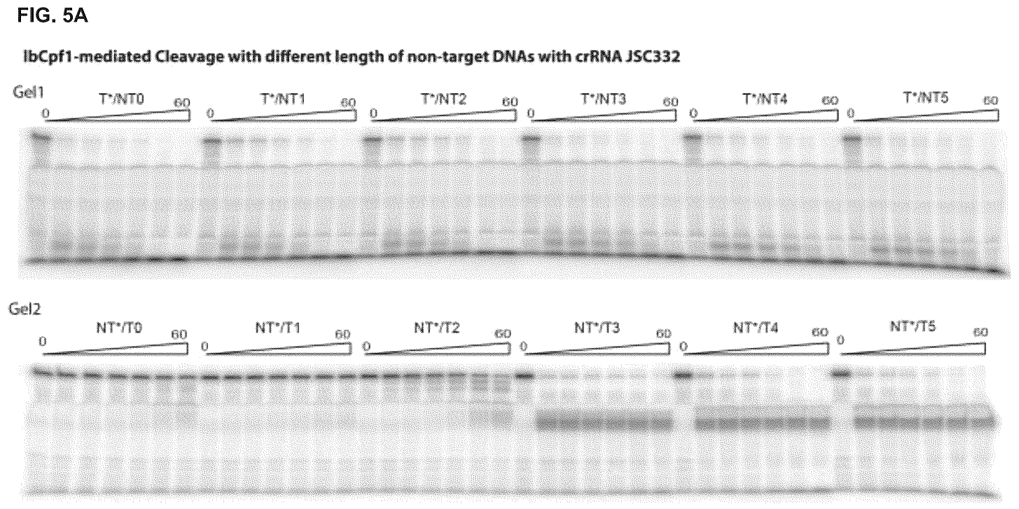

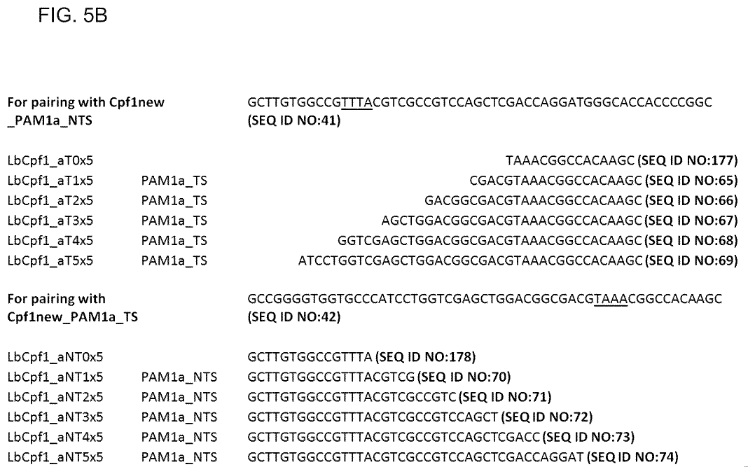

FIGS. 5A-5B. The non-target strand was cleaved only in the presence of a target strand with at least 15 nt of complementarity. LbCpf1 cleaved target strand DNA regardless of the length of the non-target strand. LbCpf1 cleaved non-target strand DNA only when the target strand had at least 15 nt of complementarity.

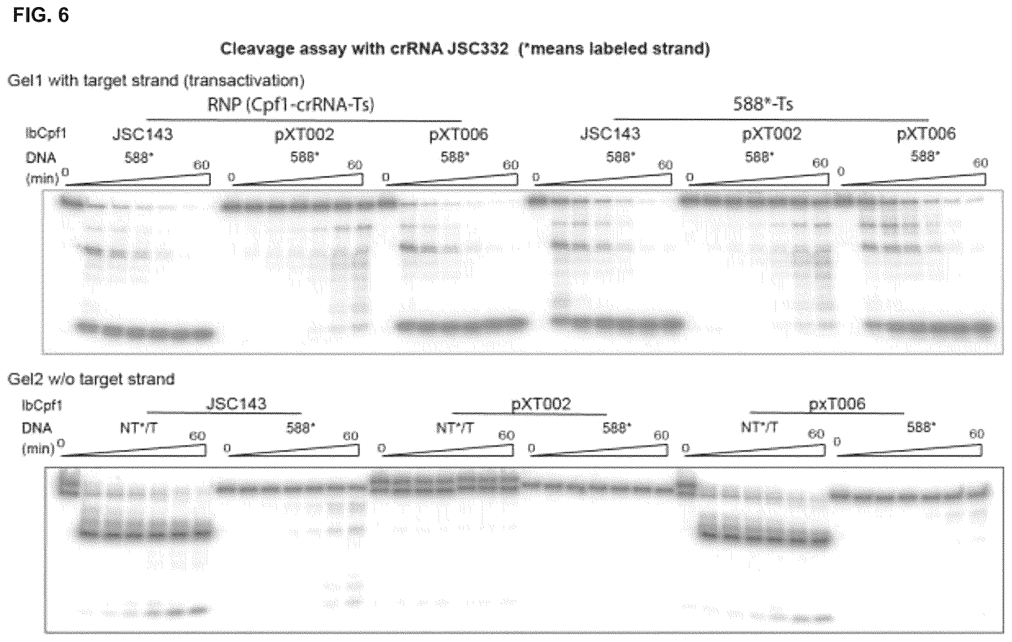

FIG. 6. The RuvC nuclease is responsible for trans-cleavage of ssDNA. In the presence of the activator target strand, non-specific trans-cleavage was not observed with a catalytically-inactive RuvC nuclease (pXT002). Trans-cleavage was still observed with a RNA-processing dead mutant (pXT006).

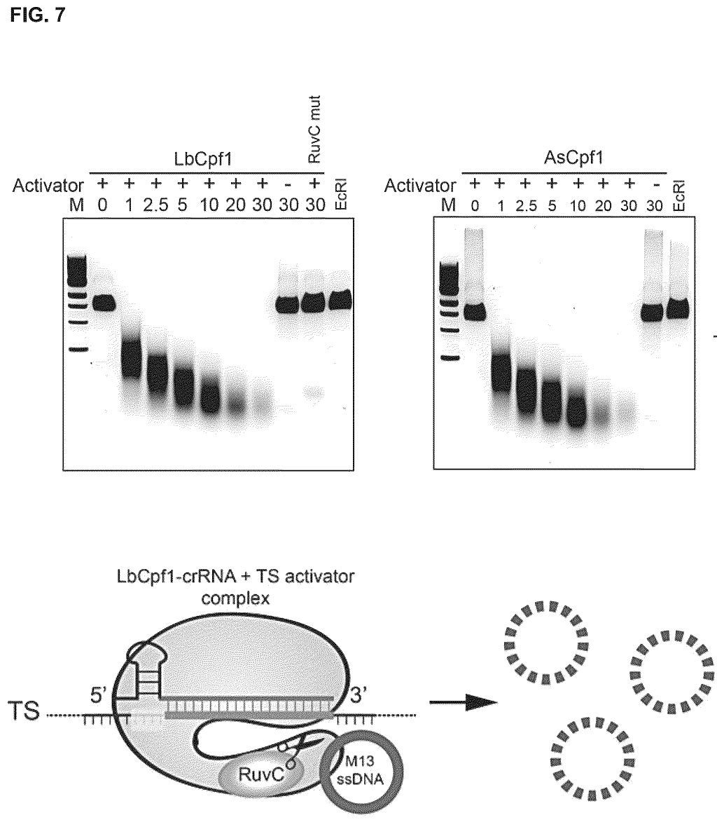

FIG. 7. Targeting by two homologs of Cas12a results in rapid “shredding” of M13 phage ssDNA. It was tested whether free 5′ or 3′ ends were required for trans-cleavage by using M13 phage circular ssDNA as a trans substrate. LbCas12a-crRNA and AsCas12a-crRNA was pre-complexed with a ssDNA activator (with no sequence complementarity to M13 phage) and incubated with M13 ssDNA at 37 C; products were resolved on a 1.5% agarose gel and visualized with SyberGold. Rapid shredding was observed at the earliest time point (1 min), and was both activator- and RuvC-dependent. The same trend with AsCas12a was observed suggesting that this activity is likely conserved across Cas12a homologs.

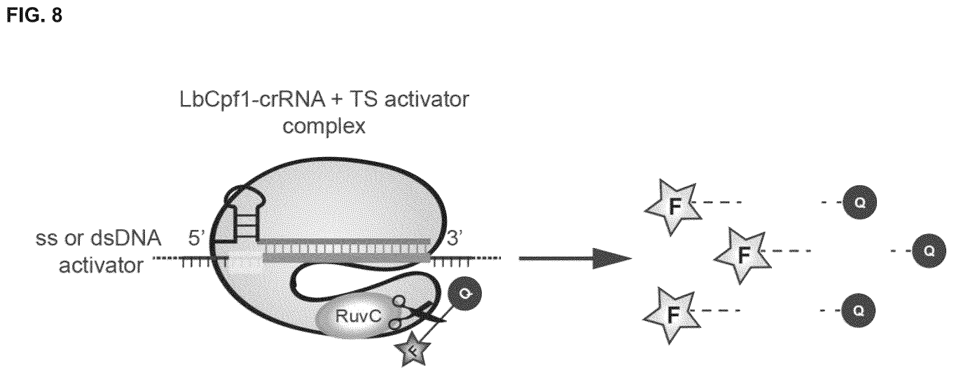

FIG. 8. Trans-cleavage by Cas12a can be detected using an FQ-based assay. To improve the throughput of measuring trans-cleavage, an FQ-based assay using a DNase Alert substrate (IDT) was adapted as a probe for trans-activation. A fluorescence signal was released upon cleavage of the substrate, which contained a DNA linker and neighboring quencher.

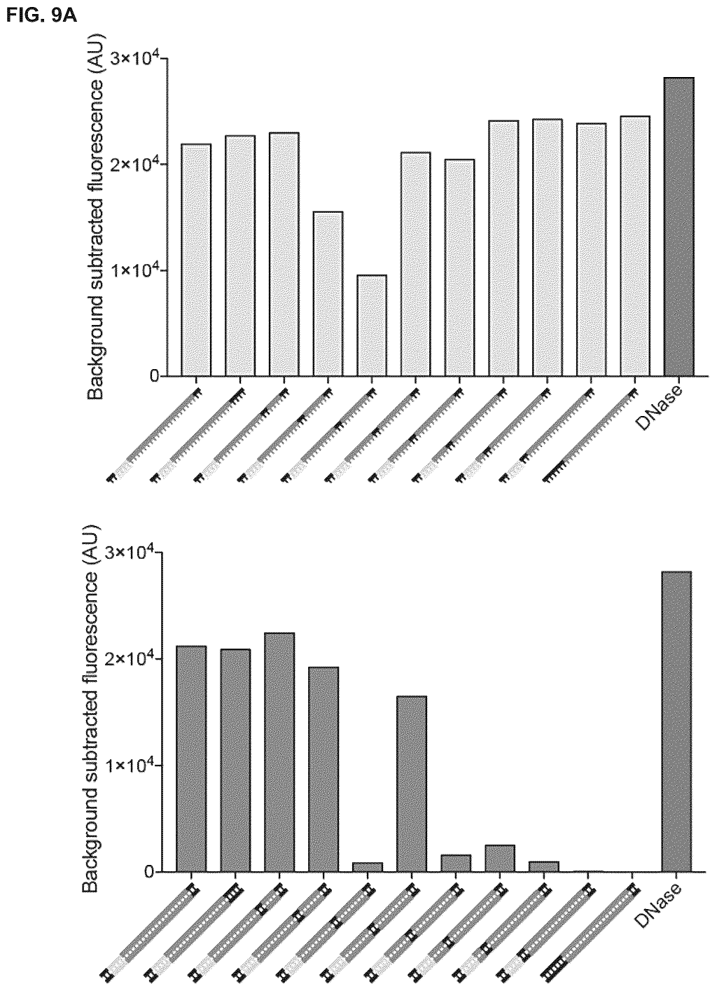

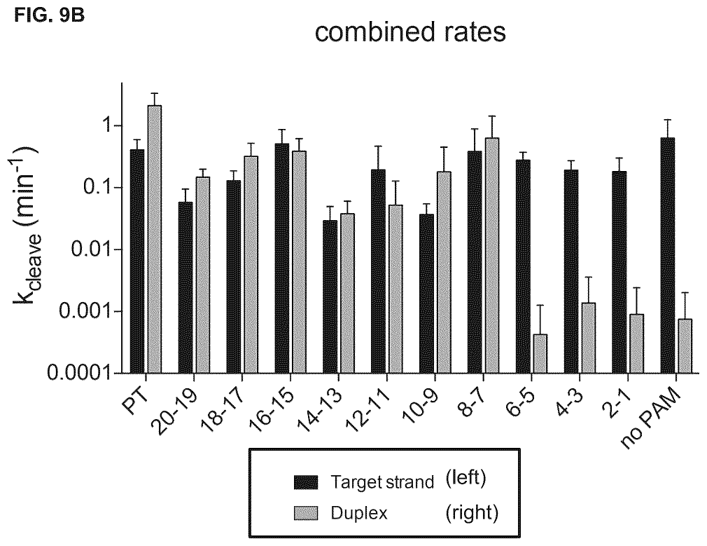

FIGS. 9A-9B. Trans-activation is sensitive to mismatches at the PAM-proximal end with duplexed substrates, but not with single-stranded substrates. Using the FQ-based assay, the mismatch tolerance for activating trans-cleavage was tested. LbCas12a-crRNA was pre-complexed with either ssDNA or dsDNA containing 2 bp mismatches from the PAM-distal to PAM-proximal end, or a mutated PAM. The top panel shows background-subtracted max fluorescence after 1 h incubation at 37 C for ssDNA (left) or dsDNA (right). The bottom panel shows observed cleavage rates after 1 h incubation at 37 C. Mismatches appeared highly tolerated in the case of ssDNA substrates, but PAM-proximal mismatches were poorly tolerated in the case of dsDNA substrates likely due to inadequate RNA strand invasion. Notably, trans-cleavage was PAM-independent when Cas12a is activated by ssDNA.

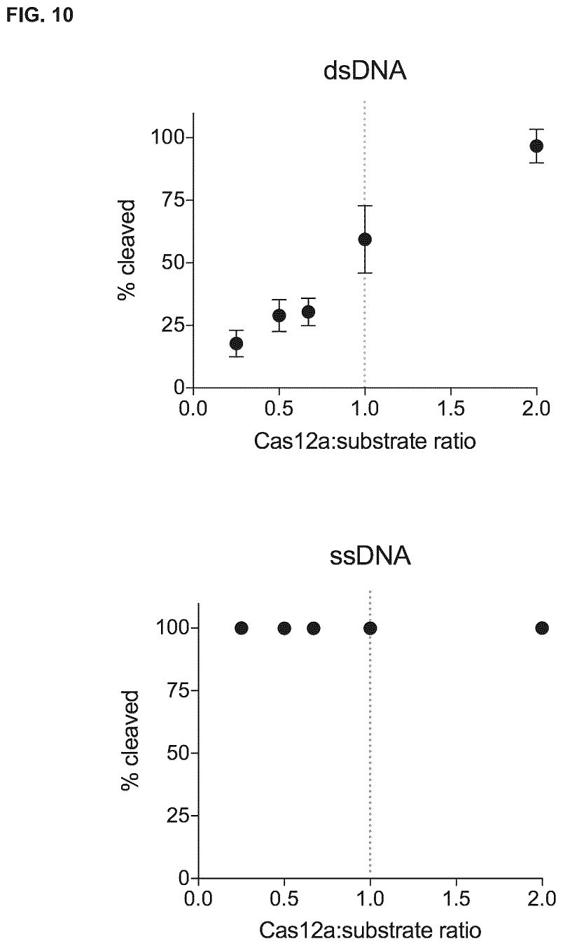

FIG. 10. dsDNA target (cis) cleavage follows single-turnover kinetics, whereas ssDNA (trans) cleavage is multiple-turnover. Turnover kinetics were assayed for cis and trans cleavage by incubating radiolabeled dsDNA (left) or random ssDNA (right) with respective LbCas12a-crRNA ratios. Each point represents quantified % cleavage after a 30 minute incubation with LbCas12a at 37 C (via denaturing PAGE). For the ssDNA kinetics, LbCas12a-crRNA was pre-complexed with ssDNA activator before addition of radiolabeled random ssDNA. The data show that cis cleavage by LbCas12a is single-turnover, whereas trans cleavage is multiple turnover.

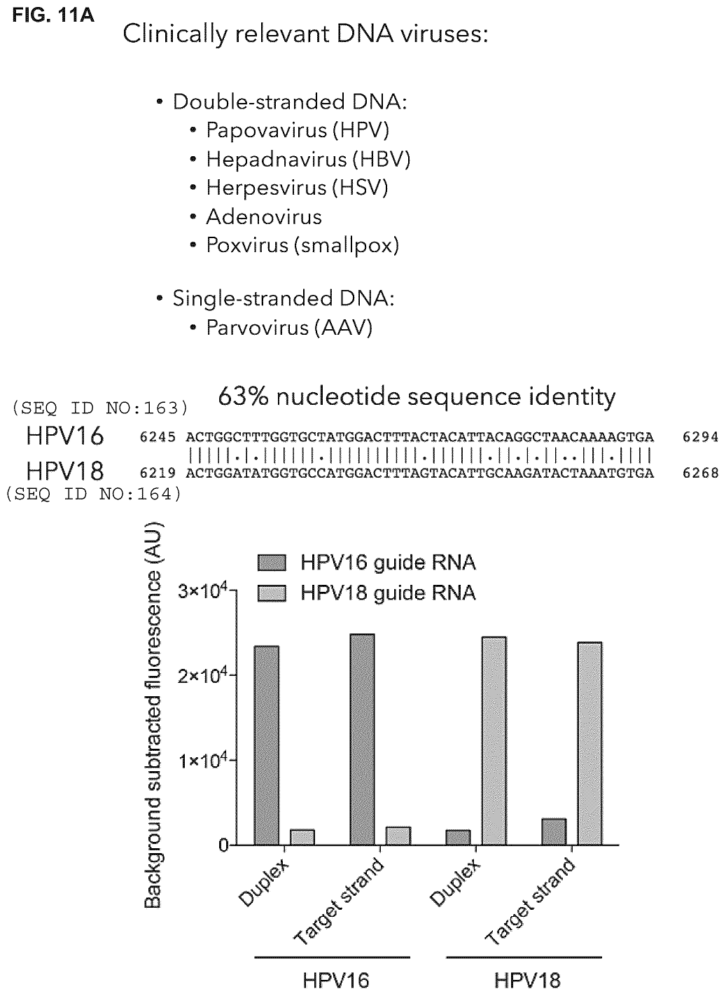

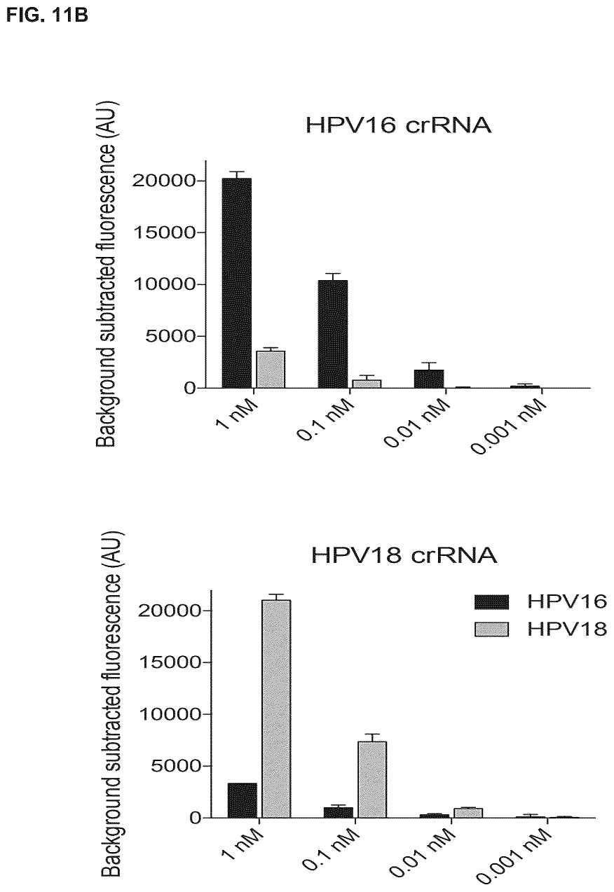

FIGS. 11A-11B. Trans-ssDNA cleavage by Cas12a can be harnessed as a simple diagnostic to distinguish viral serotypes such as HPV and other clinically relevant DNA viruses. Cas12a can detect targets as low as picomolar concentrations. To demonstrate that LbCas12a trans-activity can be harnessed as a simple diagnostic, the FQ-based assay using DNase Alert substrate was used to test whether one could distinguish two closely-related HPV sequences (HPV16 and HPV18) that are considered high-risk strains for cervical cancer. LbCas12 is pre-incubated with a crRNA targeting a HPV16 and HPV18 sequence adjacent to a TTTA PAM; the two sequences differ by 6 nucleotides. 500 bp fragments of HPV16 and HPV18 were cloned into a plasmid backbone (˜6 kb total) as a proxy for the full HPV genome (˜8 kb), and incubated with LbCas12a-crRNA for 30 min (top) or 1 h (bottom) at 37 C. HPV serotypes were easily distinguished and the method could detect down to ˜10 pM of target. This method could in principle be extended to detect any DNA virus, and examples of clinically-relevant DNA viruses are listed herein.

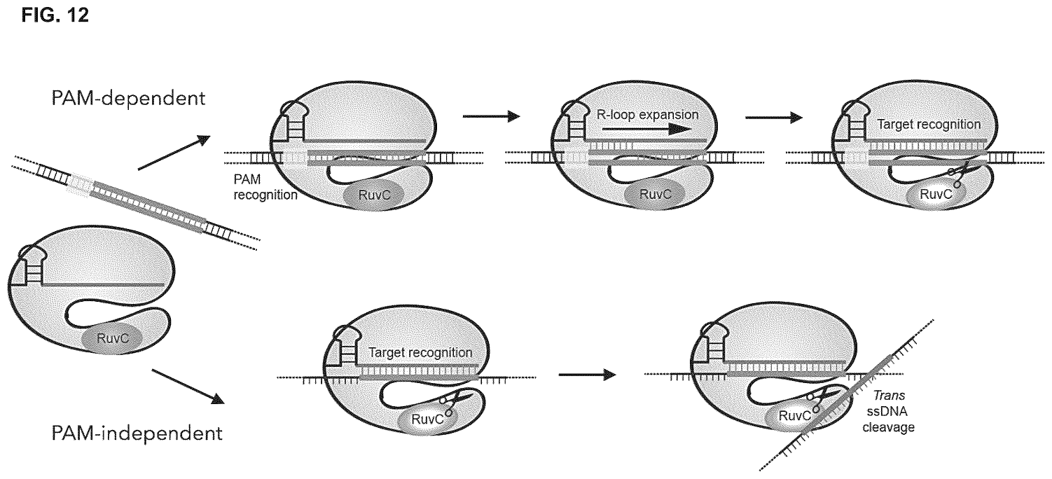

FIG. 12. A unifying model for DNA cleavage by CRISPR-Cas12a Cas12a-crRNA complex binds to a substrate in PAM-dependent (dsDNA) or PAM-independent (ssDNA) manner. When the dsDNA PAM is recognized, the duplex is interrogated by RNA strand invasion and recognition of the complementary target strand activates the RuvC nuclease to cleave both the unwound TS and NTS. Binding of the complementary ssDNA also triggers the RuvC nuclease to degrade any ssDNAs.

### Example 2

CRISPR-Cas12a (Cpf1) belongs to a family of RNA-guided DNA targeting enzymes that bind and cut DNA as components of bacterial adaptive immune systems. Like CRISPR-Cas9, Cas12a and related enzymes are also powerful genome editing tools based on their ability to induce genetic changes in cells at sites of double-stranded DNA cuts. In the course of investigating the DNA substrate selectivity of Cas12a, the inventors were surprised to find that RNA-guided DNA binding unleashes robust, non-specific single-stranded DNA (ssDNA) cleavage activity sufficient to completely degrade both linear and circular ssDNA molecules. This activity, catalyzed by the same active site responsible for site-specific dsDNA cutting, shredded ssDNA irrespective of sequence requirements and with rapid multiple-turnover cleavage kinetics. Activation of ssDNA cutting required faithful recognition of a DNA target sequence matching the guide sequence of the guide RNA with specificity sufficient to distinguish between closely related viral serotypes. The data provided herein show that Cas12a-catalyzed ssDNA degradation, not observed for CRISPR-Cas9 enzymes, is a fundamental property of other Cas12-family proteins, revealing a fascinating and surprising parallel with the RNA-triggered general RNase activity of the type VI CRISPR-Cas13 enzymes.

### Results

CRISPR-Cas adaptive immunity in bacteria and archaea uses RNA-guided nucleases to identify and cut foreign nucleic acids. The CRISPR-Cas9 family of enzymes has been widely deployed for gene editing applications in eukaryotes based on the precision of double-stranded DNA (dsDNA) cleavage induced by two catalytic domains, RuvC and HNH, at sequences complementary to a guide RNA sequence. A second family of enzymes harnessed for gene editing, CRISPR-Cas12a (formerly known as Cpf1), uses a single catalytic domain (RuvC) for guide RNA-directed dsDNA cleavage (FIG. 13A). Distinct from Cas9, Cas12a enzymes also process individual guide RNAs from a longer precursor transcript and generate dsDNA breaks with staggered 5′ and 3′ ends, features that have attracted interest in Cas12a for gene editing applications. Despite its adoption as a genome-editing tool, the substrate specificity and DNA cleavage mechanism of Cas12a are yet to be fully elucidated.

While the DNA substrate requirements for Cas12a activation were being investigated, Lachnospiraceae bacterium ND2006 Cas12a (LbaCas12a) was tested for guide RNA-directed single-stranded DNA (ssDNA) cleavage, a capability of various CRISPR-Cas9 orthologs. Purified LbaCas12a or SpyCas9 proteins were assembled with guide RNAs that have base pairing complementarity to circular, single-stranded M13 DNA phage. Although SpyCas9 catalyzed site-specific M13 cleavage, generating linear phage molecules as expected, LbaCas12a surprisingly induced rapid and complete degradation of M13 by a cleavage mechanism that could not be explained by sequence-specific DNA cutting (FIG. 13B). This robust ssDNA degradation was not observed in experiments using an LbaCas12a protein containing inactivating mutations in the RuvC catalytic domain. These results suggested that LbaCas12a possesses a unique ssDNA shredding activity that requires the same active site used for RNA-directed dsDNA cutting.

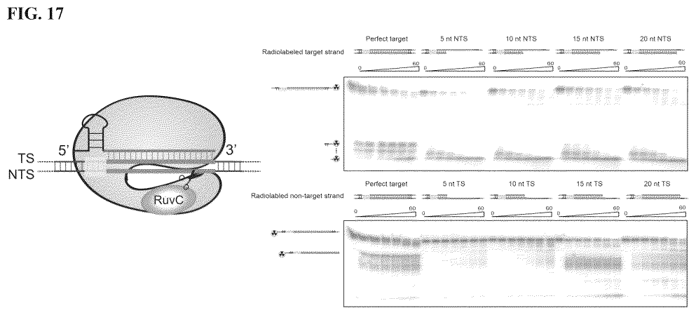

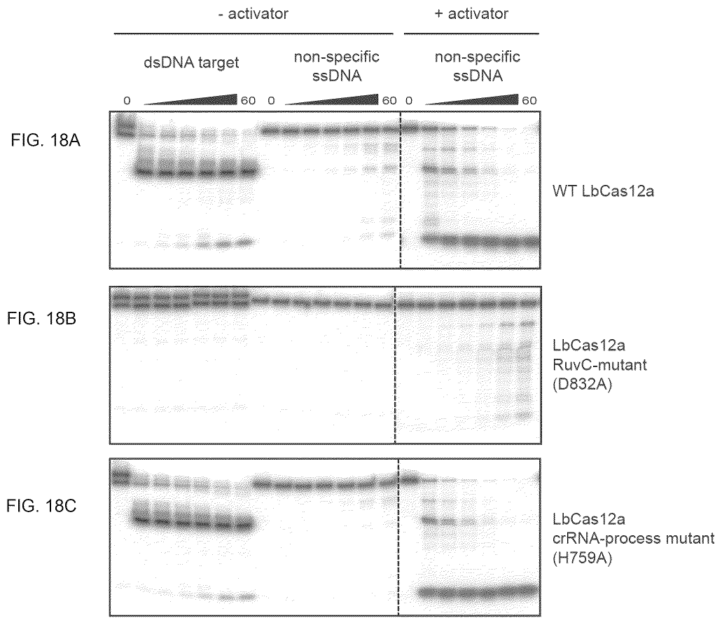

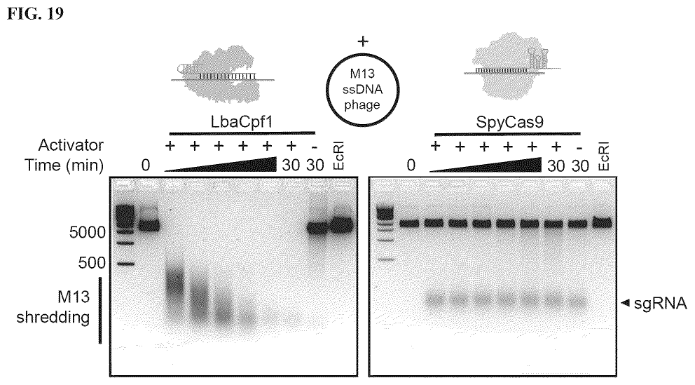

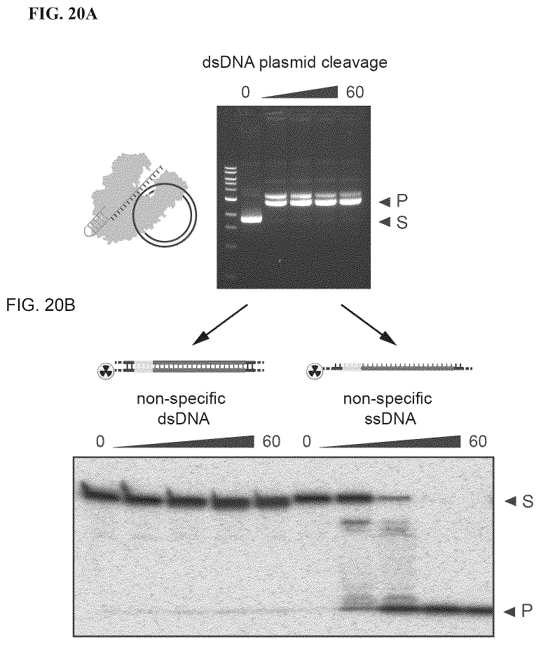

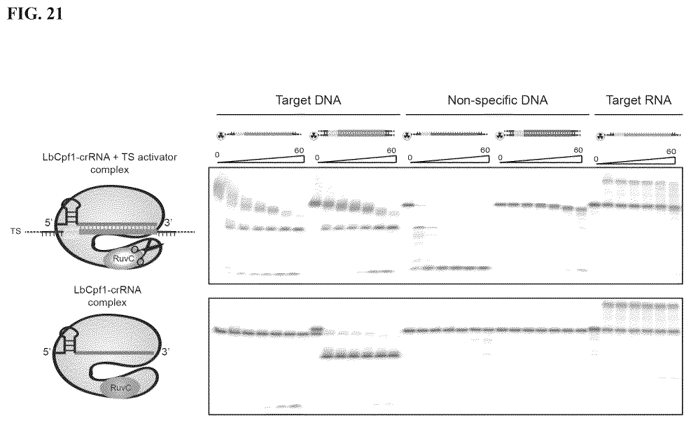

The non-target strand (NTS) was cleaved only when the target strand (TS) contained at least 15 nt of complementarity with the guide RNA (FIG. 17). These results suggested that TS recognition is a prerequisite for ssDNA cutting, raising the possibility that LbaCas12a possesses non-specific ssDNase activity. To test the idea that a TS-activated LbaCas12a could cut any ssDNA, LbaCas12a was pre-complexed with a crRNA and complementary ssDNA or dsDNA activator, and introduced an unrelated radiolabeled ssDNA, dsDNA or ssRNA in trans. Remarkably, both ssDNA and dsDNA activators triggered LbaCas12a to completely degrade the ssDNA trans-substrate to its 5′ end label in a RuvC-dependent manner (FIGS. 18A-18C, FIG. 19, FIGS. 20A-20B), whereas the dsDNA and ssRNA trans-substrates remained protected from the activated complex (FIG. 21). Together, these findings revealed that Cas12a DNA binding unleashes robust, non-specific ssDNase trans-activity by the RuvC nuclease.

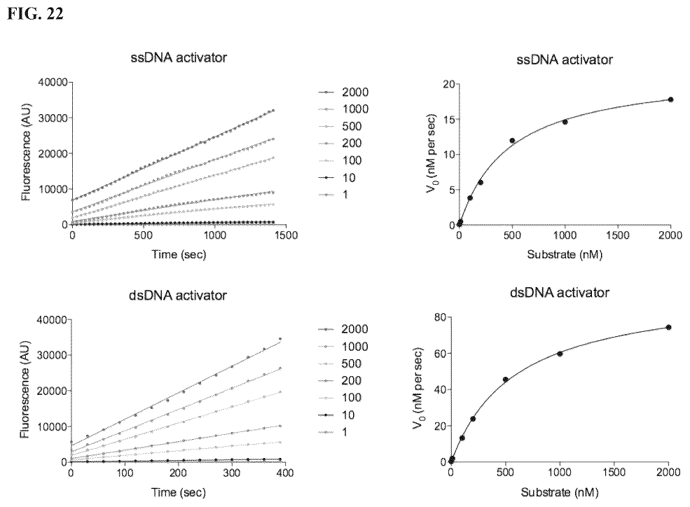

The rapid degradation of a trans substrate suggested that the kinetics of non-specific ssDNA trans-cleavage may be fundamentally different from cis-cleavage, in which LbaCas12a targets a complementary dsDNA substrate. To investigate how a single RuvC nuclease cuts by two different mechanisms, substrate turnover was observed by titrating molar ratios of either LbaCas12a-crRNA or LbaCas12a-crRNA-ssDNA activator complexes against a dsDNA target (cis) or non-specific ssDNA (trans) substrate, respectively. The fraction of cleaved target dsDNA was proportional to the molar ratio of LbaCas12a-crRNA to DNA, demonstrating that cis-cleavage is single-turnover (FIG. 14A). In contrast, the fraction of cleaved non-specific ssDNA was saturated at sub-equimolar ratios, revealing that trans-cleavage follows multiple turnover kinetics (FIG. 14B). To further examine the Michaelis-Menten kinetics of trans-cleavage, a real-time, fluorophore quencher (FQ)-labeled DNA reporter assay was adapted to measure non-specific DNase activity under conditions where LbaCas12a-crRNA is stably bound to a ssDNA or dsDNA activator. LbaCas12a pre-complexed with a ssDNA activator revealed a highly robust activity that yielded a catalytic efficiency (k_{cat}/Kₘ) of 5.1×10⁸ s⁻¹ M⁻¹. When pre-complexed with a dsDNA activator, the catalytic efficiency was nearly an order of magnitude faster and approached the rate of diffusion with a k_{cat}/Kₘ measurement of 1.7×10⁹ s⁻¹ M⁻¹ (FIG. 14C, FIG. 22). These differences in catalytic efficiencies suggest a potential role for the NTS of the dsDNA activator to stabilize the Cas12a complex in an optimal conformation for cutting a trans-ssDNA substrate.

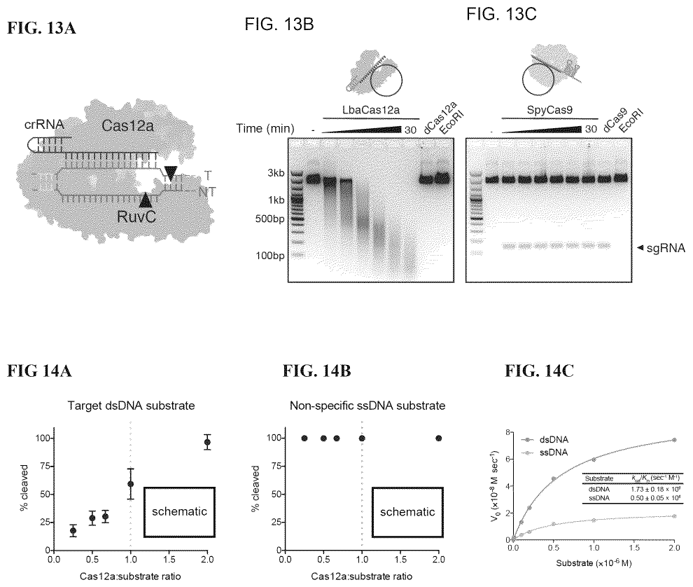

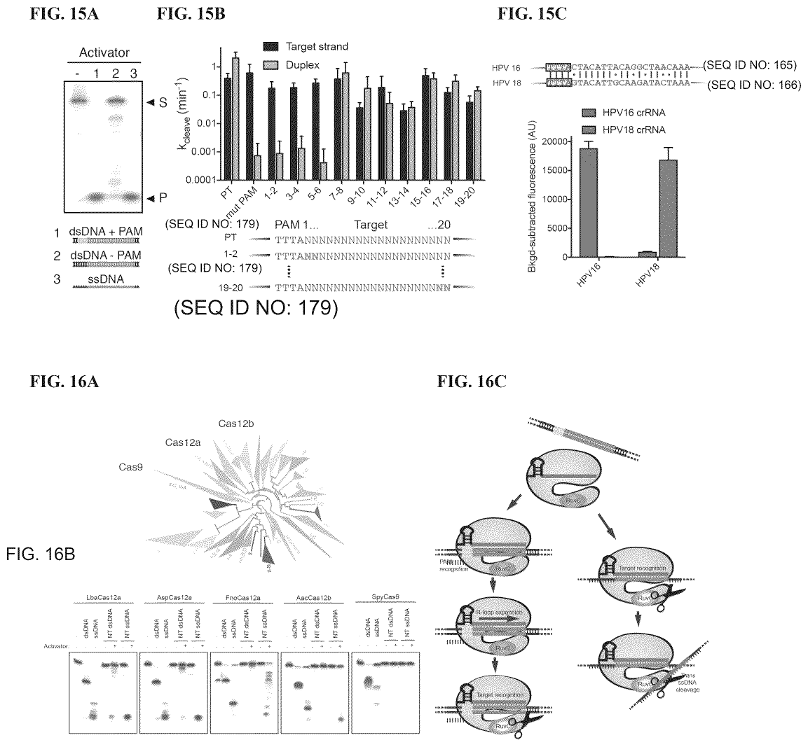

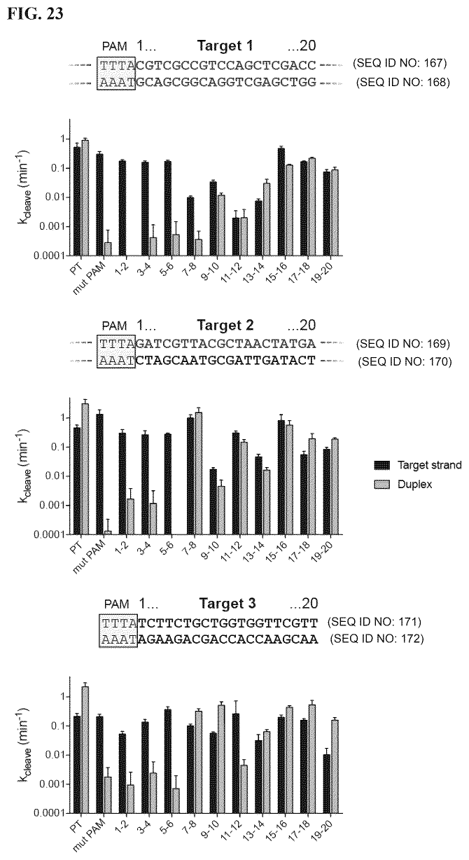

The substrate specificity of a ssDNA versus dsDNA activator for trans-cleavage was next considered. First, experiments were performed to confirm that PAM recognition is critical for activation by a complementary dsDNA but not for a matching ssDNA, consistent with the requirements for target binding (FIG. 15A). To test whether mismatches along the activator sequence could impact the rate of trans-cleavage, two base-pair (bp) mismatches were introduced across the target sequence in either a ssDNA or dsDNA activator. Using the FQ-based assay, LbaCas12a was pre-loaded with the crRNA and activator before addition of the ssDNA reporter, and the real-time increase in fluorescence signal was measured as a proxy for the observed trans-cleavage rate. Whereas mismatches across the ssDNA activator sequence were generally well tolerated, mismatches in the PAM or “seed region” of the dsDNA activator were poorly tolerated (FIG. 15B, FIG. 23). These trends using a dsDNA activator suggest that PAM recognition and unidirectional DNA unwinding provide additional regulation for trans-cleavage. However, extensive base complementary between the crRNA and target strand is the only requirement for activating trans-cleavage.

Because LbaCas12a demonstrated higher specificity using dsDNA activators for trans-cleavage, the FQ-based assay was used to test whether LbaCas12a could be readily programmed to distinguish between two closely-related dsDNA viruses. As a proof-of-principle, the Human papillomavirus (HPV) serotypes 16 (HPV16) and 18 (HPV18) were selected, which account for approximately 70% of all cases of cervical cancer following persistent HPV infection. LbaCas12a was first pre-complexed with a crRNA targeting an HPV16 or HPV18 sequence adjacent to a TTTA PAM that differ by only 6 nucleotides (FIG. 15C). As a proxy for the full HPV genome (˜8 kb), 500 bp fragments of HPV16 and HPV18 were cloned into a ˜5 kb plasmid, and incubated the HPV-containing plasmid with LbaCas12a-crRNA. Robust activation of trans-cleavage was observed only when LbaCas12a was in the presence of at least ˜10 pM of its cognate HPV target (FIG. 15D, FIG. 24), suggesting that the native specificity of dsDNA recognition and trans-cleavage activation by LbaCas12a could in principle be extended to detect any dsDNA virus.

It was then tested whether this trans-cleavage activity might be conserved among the Cas12a family, and even more broadly across evolutionarily distinct type V effector proteins. Two lines of evidence hinted at this possibility: first, target-bound crystal structures of Cas12b (previously known as C2c1) suggested that its RuvC catalytic pocket accommodates both the TS and the NTS for cleavage, similar to the cis-cleavage mechanism proposed for Cas12a. Second, despite low sequence and structural similarity between these subtypes, a unifying structural feature among all Cas12 proteins is the RuvC nuclease domain near the C-terminal end of the polypeptide. Therefore, two additional Cas12a orthologs from Acidaminococcus sp. (AspCas12a) and Francisella novicida (FnoCas12a) were selected, as well as a Cas12b protein from Alicyclobacillus acidoterrestris (AacCas12b) to test for cis- and trans-cleavage (FIG. 16A). Despite varying efficiencies, all of the homologs evaluated demonstrated non-specific ssDNase activity when pre-complexed with a complementary ssDNA activator (FIG. 16B), suggesting that trans-cleavage is a fundamental property of Cas12-family proteins. These experiments further underscore the functional convergence of trans-cleavage between the DNA-targeting type V and RNA-targeting type VI effector proteins.

The data herein suggest a new mechanism for target interference by the Cas12 protein family and a new model is proposed herein in which the Cas12-guide RNA complex binds to a DNA substrate in a PAM-dependent (dsDNA) or PAM-independent (ssDNA) manner (FIG. 16C). Following PAM recognition for a dsDNA substrate, RNA strand invasion and target recognition activates the RuvC nuclease to cleave the unwound TS and trim back the NTS, thereby generating the staggered dsDNA break and robustly activating ssDNA trans-cleavage. Binding of a complementary ssDNA bypasses PAM recognition and RNA strand invasion, but is sufficient to trigger the RuvC nuclease to degrade any ssDNAs.

### Example 3: CRISPR-Cas12a Target Binding Unleashes Indiscriminate Single-Stranded DNase Activity

The data presented here show that RNA-guided DNA binding unleashes robust, indiscriminate single-stranded DNA (ssDNA) cleavage activity in Cas12 proteins (e.g., Cas12a) sufficient to completely degrade both linear and circular ssDNA molecules. The data show that target-activated non-specific ssDNase activity, catalyzed by the same active site responsible for site-specific dsDNA cutting, is a fundamental property of type V CRISPR-Cas12 enzymes. Activation of ssDNA cutting requires faithful recognition of a DNA target sequence matching the guide sequence of the guide RNA with specificity capable of distinguishing closely related DNA sequences. Target-dependent Cas12 ssDNase activation was combined with isothermal amplification to create a method termed DNA Endonuclease Targeted CRISPR Trans Reporter (DETECTR), which achieved attomolar sensitivity for nucleic acid detection. DETECTR is demonstrated here to facilitate rapid and specific detection of DNA (e.g., HPV) in human patient samples, thereby providing a simple platform for nucleic acid-based, point-of-care diagnostics.

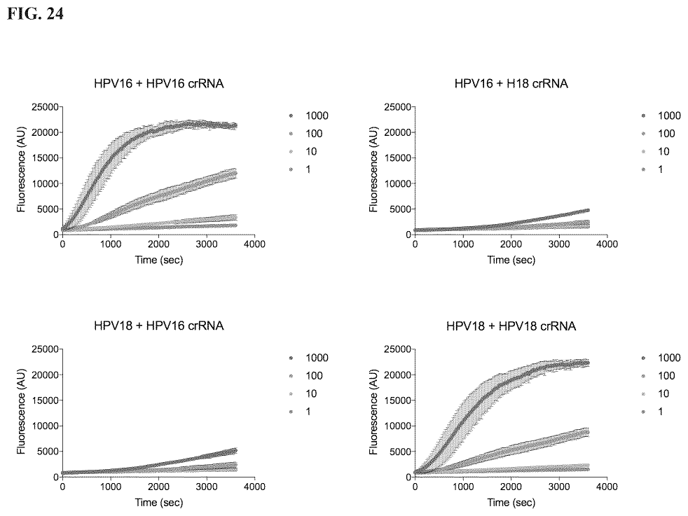

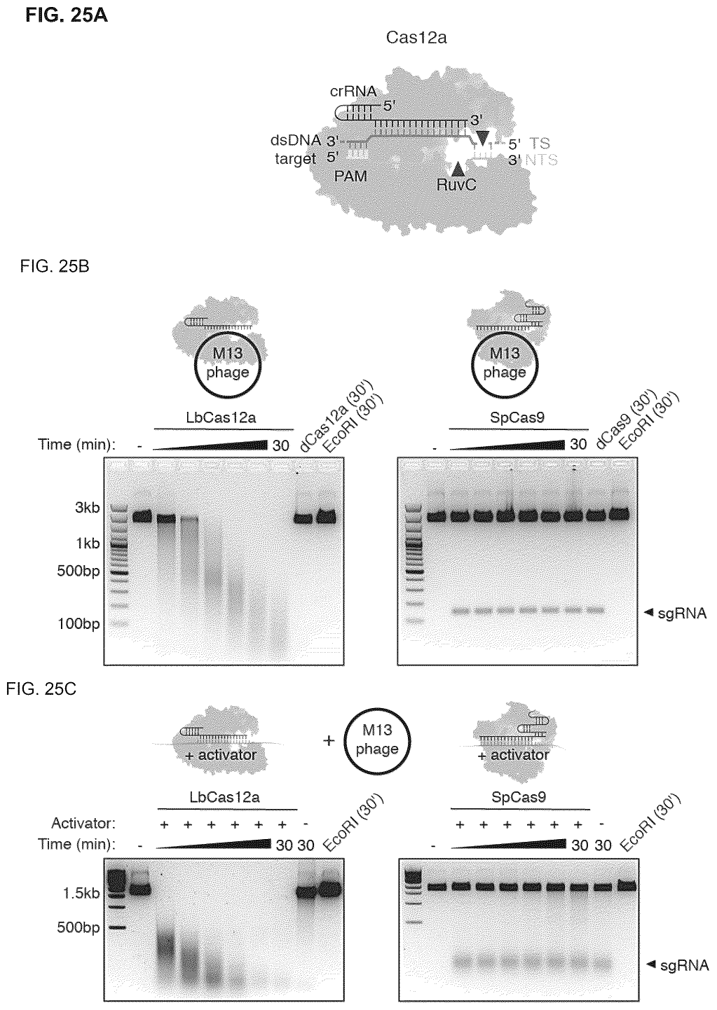

CRISPR-Cas adaptive immunity in bacteria and archaea uses RNA-guided nucleases to target and degrade foreign nucleic acids. The CRISPR-Cas9 family of proteins has been widely deployed for gene editing applications based on the precision of double-stranded DNA (dsDNA) cleavage induced by two catalytic domains, RuvC and HNH, at sequences complementary to a guide RNA sequence. A second family of enzymes, CRISPR-Cas12a (Cpf1), uses a single RuvC catalytic domain for guide RNA-directed dsDNA cleavage (FIG. 25A). Distinct from Cas9, Cas12a enzymes recognize a T-rich protospacer adjacent motif (PAM), catalyze their own guide RNA (crRNA) maturation and generate a PAM-distal dsDNA break with staggered 5′ and 3′ ends, features that have attracted interest for gene editing applications.

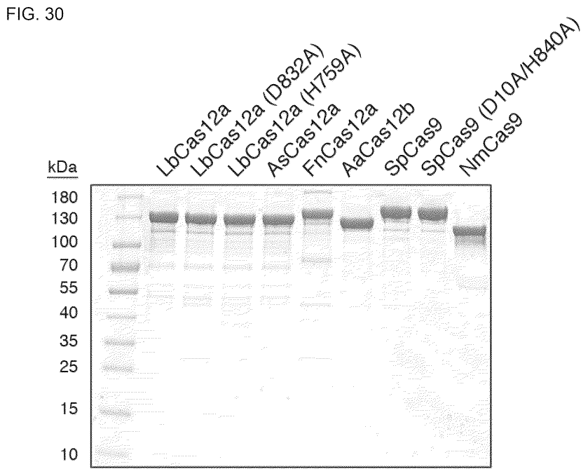

While investigating substrate requirements for Cas12a activation, Lachnospiraceae bacterium ND2006 Cas12a (LbCas12a) was tested for guide RNA-directed single-stranded DNA (ssDNA) cleavage, a capability of diverse CRISPR-Cas9 orthologs. Purified LbCas12a or Streptococcus pyogenes Cas9 (SpCas9) proteins (FIG. 30) were assembled with guide RNA sequences targeting a circular, single-stranded M13 DNA phage. In contrast to SpCas9, it was surprising to find that LbCas12a induced rapid and complete degradation of M13 by a cleavage mechanism that could not be explained by sequence-specific DNA cutting (FIG. 25B). This ssDNA shredding activity, not observed using an LbCas12a protein containing an inactivating mutation in the RuvC catalytic domain (D832A), raised the possibility that a target-bound LbCas12a could degrade any ssDNA, regardless of complementarity to the guide RNA. To test this idea, LbCas12a or SpCas9 was assembled with a different guide RNA and its complementary ssDNA that has no sequence homology to M13 phage genome sequence, and single-stranded M13 DNA was added to the reaction. Remarkably, LbCas12a catalyzed M13 degradation only in the presence of this complementary ssDNA “activator”, an activity not observed for SpCas9 (FIG. 25C). These findings revealed that binding of the LbCas12a-crRNA complex to a guide-complementary ssDNA unleashed robust, non-specific ssDNA trans-cleavage activity.

FIGS. 25A-25C. Cas12a target recognition activated non-specific single-stranded DNA cleavage. FIG. 25A. Cas12a-crRNA complex binds a dsDNA substrate and generates a 5′ overhang staggered cut using a single RuvC nuclease. FIGS. 25B-25C. Representative M13 ssDNA cleavage timecourses with purified LbCas12a (left) and SpCas9 (right) complexed with a FIG. 25B guide RNA complementary to M13 phage or FIG. 25C a guide RNA and complementary ssDNA activator with no sequence homology to M13 phage.

FIG. 30. Purification of Cas12 and Cas9 proteins. SDS-PAGE gel of all purified Cas12 and Cas9 proteins used in this study.

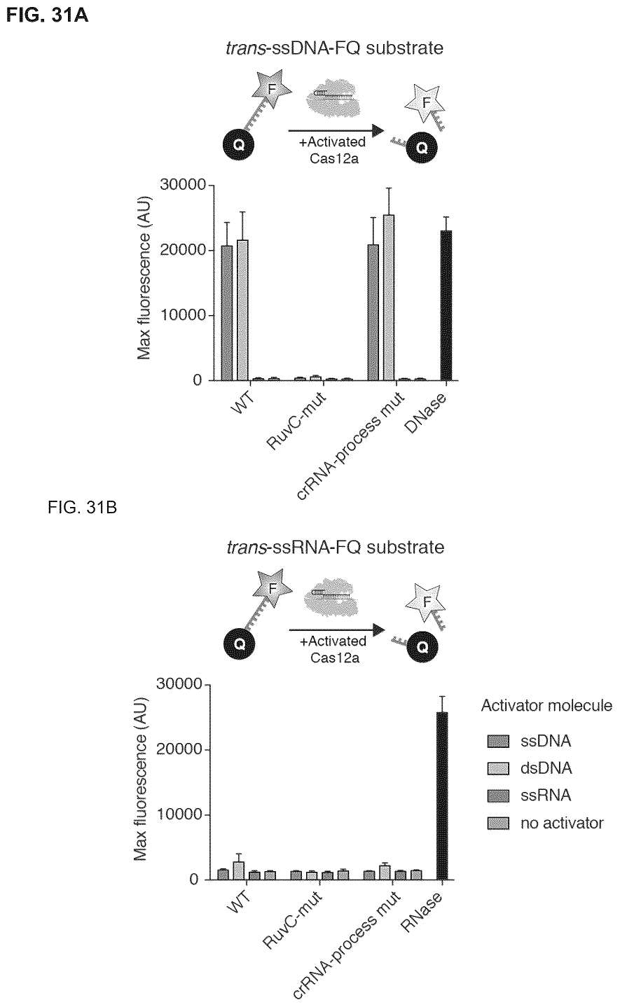

The requirements for LbCas12a-catalyzed trans-cleavage activity was next investigated. Using a fluorophore quencher (FQ)-labeled reporter assay, LbCas12a was assembled with its crRNA and either a complementary ssDNA, dsDNA or single-stranded RNA (ssRNA), and an unrelated ssDNA- or ssRNA-FQ reporter was introduced in trans (FIGS. 31A-31B). Both the crRNA-complementary ssDNA or dsDNA (the activator) triggered LbCas12a to cleave the ssDNA-FQ reporter substrate (FIG. 31A). However, ssRNA was neither capable of activating trans-cleavage nor susceptible to degradation by LbCas12a (FIG. 31B) confirming that LbCas12a harbors a DNA-activated general DNase activity.

FIGS. 31A-31B. LbCas12a is a DNA-activated general DNase. Quantification of maximum fluorescence signal generated after incubating LbCas12a-crRNA-activator with a custom FIG. 31A trans-ssDNA-FQ or FIG. 31B trans-ssRNA-FQ reporter for 1 h at 37° C., with DNase I or RNase A controls where indicated. Error bars represent the mean±s.d., where n=3 replicates.

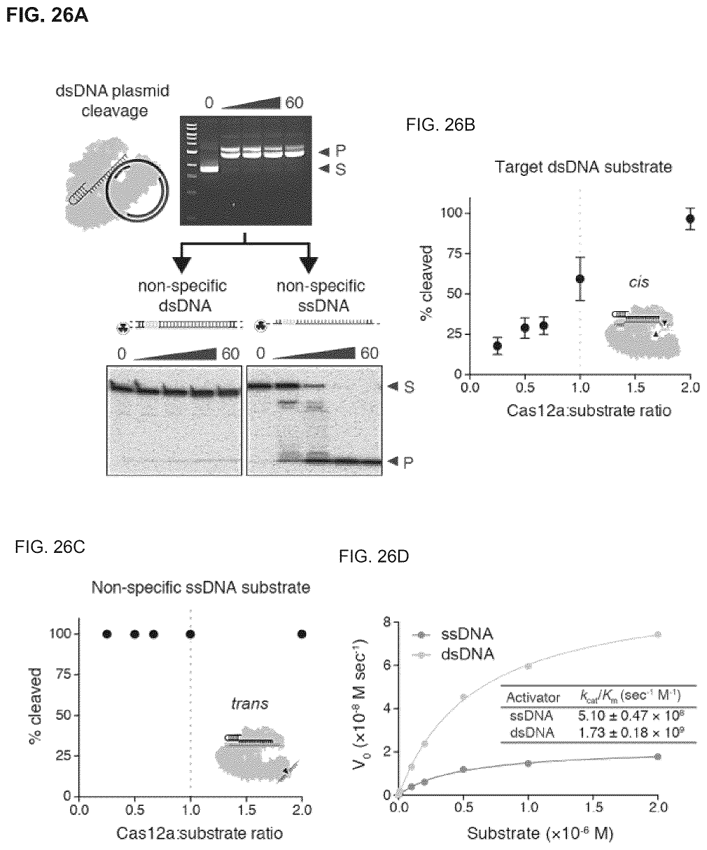

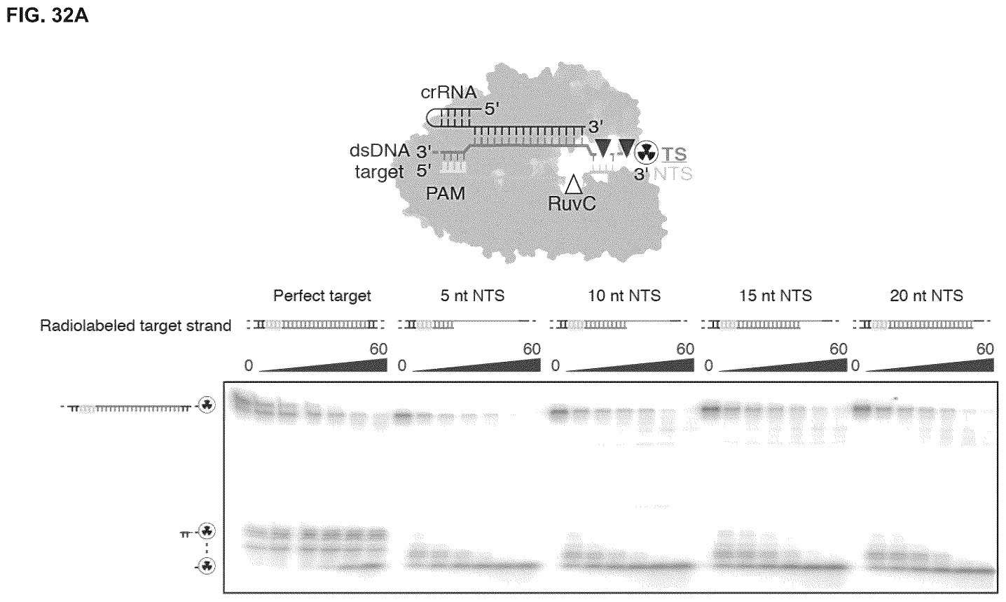

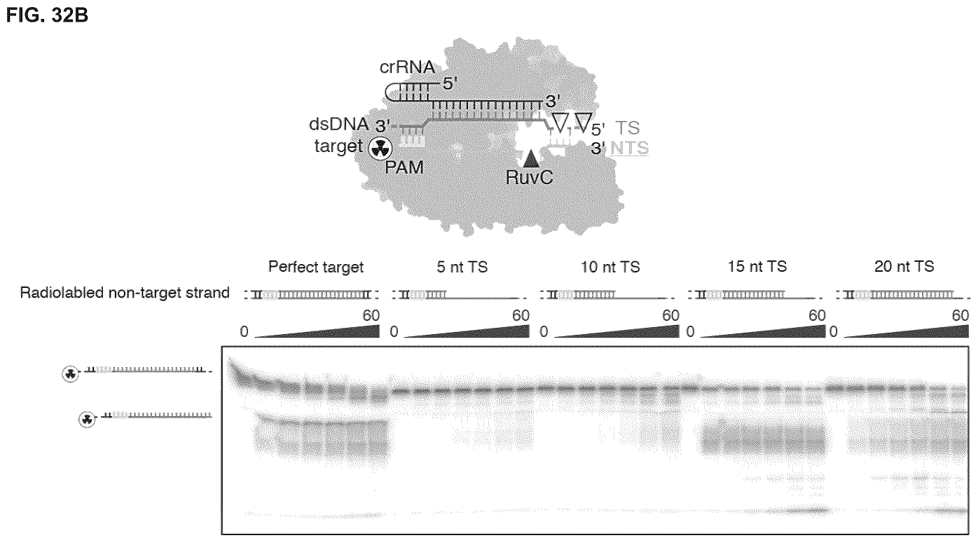

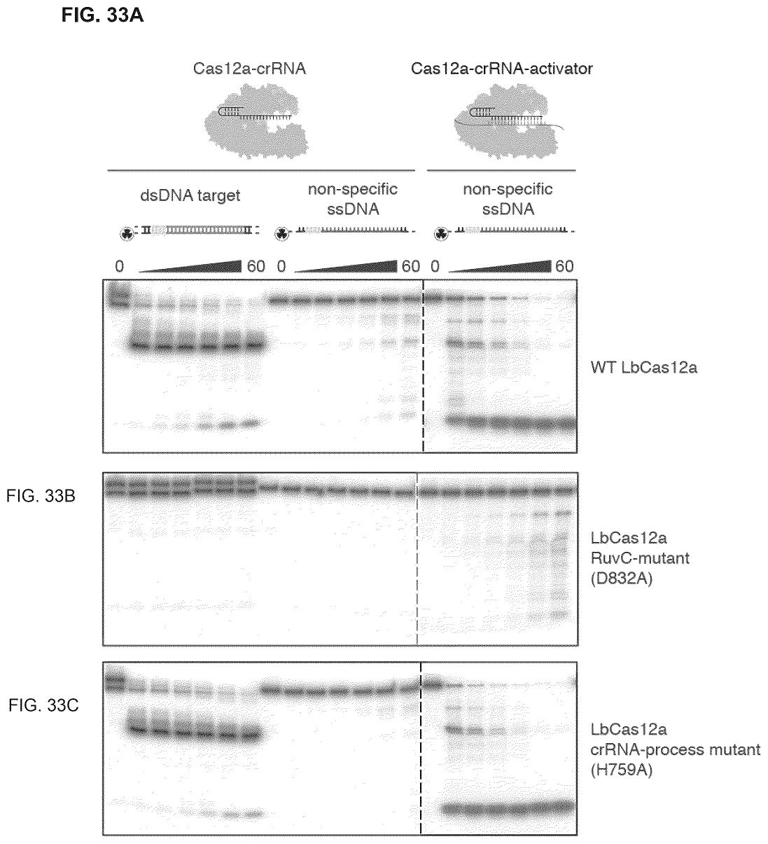

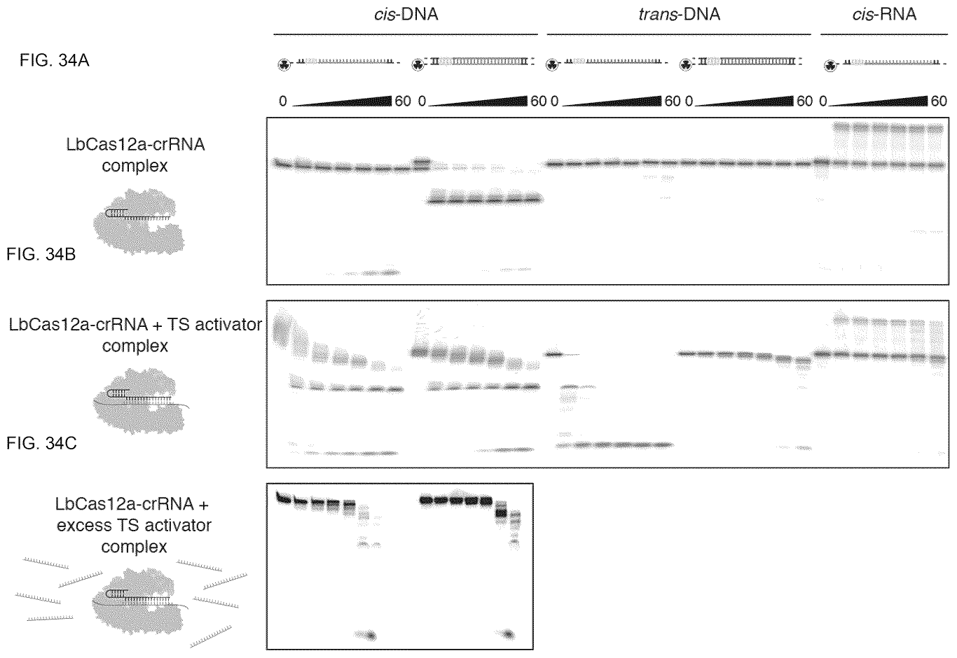

To determine how LbCas12a-catalyzed ssDNA cleavage activity relates to site-specific dsDNA cutting, the length requirements of both the target strand (TS) and non-target strand (NTS) for LbCas12a activation was tested using radiolabeled oligonucleotides. Although TS cutting occurred irrespective of the NTS length (FIG. 32A), NTS cleavage occurred only when the TS contained at least 15 nucleotides (nt) of complementarity with the crRNA (FIG. 32B). This showed that TS recognition is a prerequisite for NTS cutting. To test whether LbCas12a remains active for non-specific ssDNA cleavage after sequence-specific binding and cleavage of a dsDNA substrate, a dsDNA plasmid was first cut with an LbCas12a-crRNA complex, and then an unrelated dsDNA or ssDNA was added to the reaction (FIG. 26A). Whereas the non-specific dsDNA substrate remained intact, the ssDNA was rapidly degraded in a RuvC-domain dependent manner (FIG. 26A; FIGS. 33A-33C; FIGS. 34A-34C). Using truncated activators that are too short to be cleaved, it was next determined that only target DNA binding is required to activate trans-ssDNA cleavage (FIGS. 35A-35B). Together, these results show that RNA-guided DNA binding activates LbCas12a for both site-specific dsDNA cutting and non-specific ssDNA trans-cleavage.

FIGS. 26A-26D. Kinetics of Cas12a ssDNA trans-cleavage. FIG. 26A Sequence-specific plasmid DNA cleavage reactions by LbCas12a-crRNA (top) were introduced to a separate radiolabeled dsDNA or ssDNA substrate of unrelated sequence (bottom); timecourses represent minutes. FIG. 26B Target dsDNA or FIG. 26C non-specific ssDNA incubated with molar ratios of LbCas12a-crRNA as indicated. Each point represents the mean quantified percent cleavage after 30 minutes at 37° C., at which time the reaction was at completion. Error bars represent the mean±s.d., where n=3 replicates. FIG. 26D Representative Michaelis-Menten plot for LbCas12a-catalyzed ssDNA trans-cleavage using a dsDNA or ssDNA activator. Measured k_{cat}/Kₘ values report mean±s.d., where n=3 replicates.

FIGS. 32A-32B. Target strand recognition is a pre-requisite for single-stranded DNA cleavage. Cleavage timecourse assays using LbCas12a with FIG. 32A truncated non-target strand (NTS) annealed to a radiolabeled target strand (TS), FIG. 32B truncated TS annealed to a radiolabeled NTS. Timecourses represent minutes and cleavage products resolved by denaturing PAGE. Schematic on right depicts cleavage of the radiolabeled TS FIG. 32A or NTS FIG. 32B, which generates a Cas12a-mediated staggered cut.

FIGS. 33A-33C. The RuvC nuclease domain is responsible for activator-dependent, non-specific DNase activity. Cleavage timecourse gel with radiolabeled non-target strand of a complementary dsDNA and non-specific ssDNA substrate using FIG. 33A WT LbCas12a, FIG. 33B RuvC catalytic mutant (D832A) and FIG. 33C crRNA-processing mutant (H759A), with or without a ssDNA activator. Timecourses represent minutes and cleavage products were resolved by denaturing PAGE.

FIGS. 34A-34C. LbCas12a trans-cleavage degrades complementary and non-specific ssDNA, but not ssRNA. Cleavage timecourse gels of LbCas12a-crRNA complexes using FIG. 34A no activator, FIG. 34B ssDNA activator in 1.2-fold molar excess, or FIG. 34C ssDNA activator in 100-fold molar excess. Radiolabeled substrates are indicated, where cis indicates a complementary target and trans indicates a non-complementary sequence. For cis substrates, the non-target strand was radiolabeled. Timecourses represent minutes and cleavage products were resolved by denaturing PAGE.

FIGS. 35A-35B. Target strand cleavage by Cas12a is not required for triggering non-specific ssDNase activity. Cleavage timecourse assays using LbCas12a with FIG. 35A radiolabeled target strand with either a ssDNA (10-25 nt) or dsDNA (10-25 bp) substrate, or FIG. 35B radiolabeled non-specific ssDNA substrate in the presence of either a ssDNA (10-25 nt) or dsDNA (10-25 bp) activator. Timecourses represent minutes and cleavage products were resolved by denaturing PAGE.

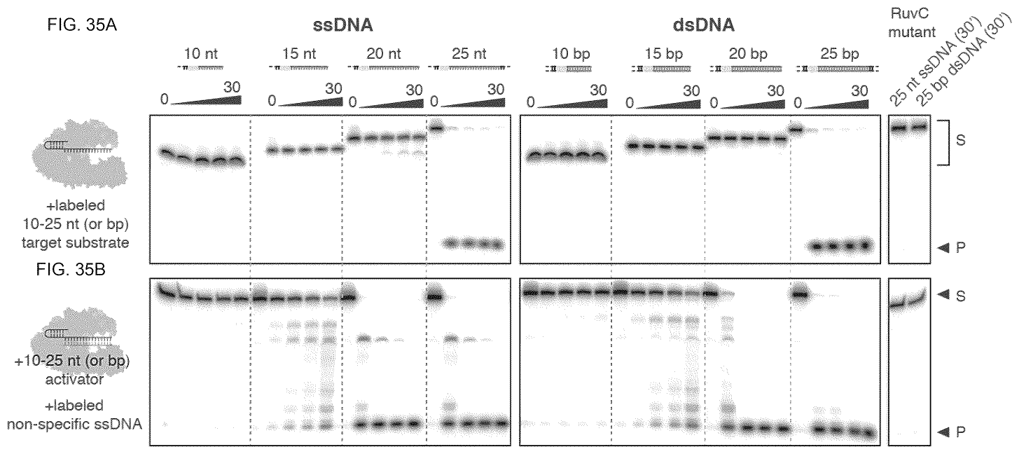

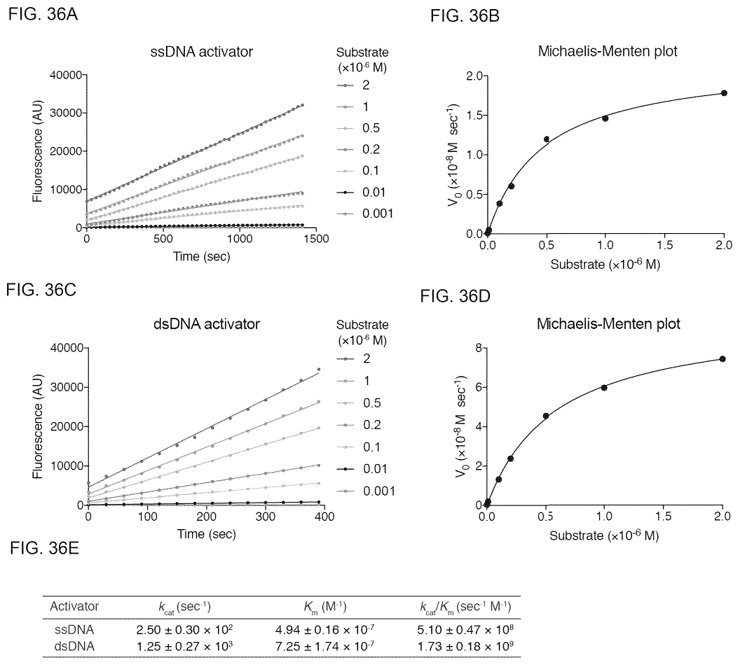

The rapid degradation of a trans substrate suggested that the kinetics of LbCas12a-catalyzed site-specific dsDNA (cis-) cleavage and non-specific ssDNA (trans-) cleavage are fundamentally different. Stoichiometric titration experiments showed that cis-cleavage is single-turnover (FIG. 26B), whereas trans-cleavage is multiple-turnover (FIG. 26C). Although the Cas12a-crRNA complex remains bound to the dsDNA target following cis-cleavage, the complex releases its PAM-distal cleavage products from the RuvC active site, enabling ssDNA substrate access and turnover. Using the FQ assay, it was found that LbCas12a-crRNA bound to a ssDNA activator molecule catalyzed trans-ssDNA cleavage at a rate of ˜250 per second and a catalytic efficiency (k_{cat}/Kₘ) of 5.1×10⁸ s⁻¹ M⁻¹. When bound to a dsDNA activator, LbCas12a-crRNA catalyzed ˜1250 turnovers per second with a catalytic efficiency approaching the rate of diffusion with a k_{cat}/Kₘ of 1.7×10⁹ s⁻¹ M⁻¹ (FIG. 26D; FIGS. 36A-36E). These differences suggested that the NTS of the dsDNA activator helps stabilize the Cas12a complex in an optimal conformation for trans-ssDNA cutting.

FIGS. 36A-36E. Michaelis-Menten analysis revealed robust trans-cleavage activity with a ssDNA and dsDNA activator. Representative plots of initial velocity versus time for a FIG. 36A ssDNA or FIG. 36C dsDNA activator, using 0.1 nM effective LbCas12a-crRNA-activator complex and increasing DNaseAlert substrate concentrations at 37° C. Michaelis-Menten fits for the corresponding FIG. 36B ssDNA or FIG. 36D dsDNA activator. FIG. 36E Calculated k_{cat}, Kₘ and k_{cat}/Kₘ values report the mean±s.d., where n=3 replicates.

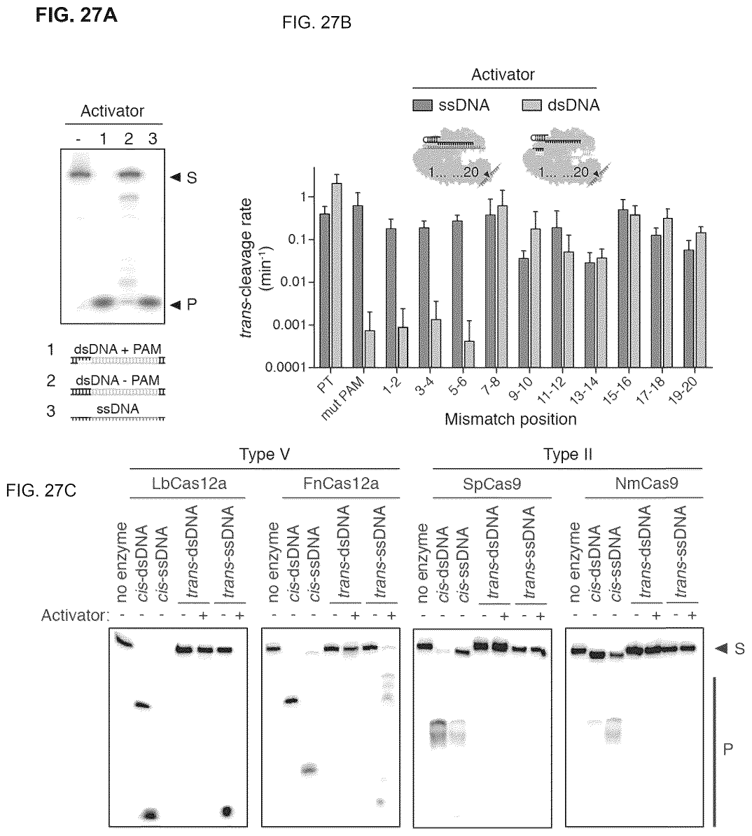

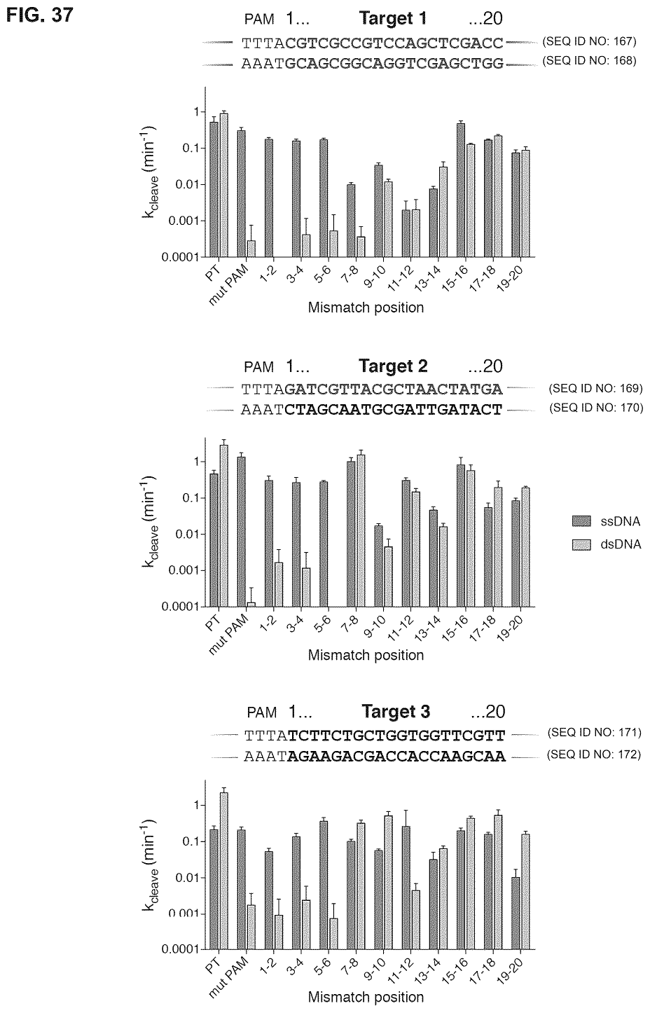

The specificity of trans-cleavage activation was next tested using either a ssDNA or dsDNA activator. The PAM sequence required for dsDNA binding by CRISPR-Cas12a was found to be critical for catalytic activation by a crRNA-complementary dsDNA, but not for a crRNA-complementary ssDNA (FIG. 27A). Two base-pair (bp) mismatches introduced along the crRNA-complementary sequence of either a ssDNA or dsDNA activator molecule slowed the trans-cleavage rate of a ssDNA-FQ reporter by up to ˜100 fold, depending on the mismatch position. For only the dsDNA activator, alterations to the PAM sequence or mismatches between the crRNA and PAM-adjacent “seed region” also had large inhibitory effects on trans-ssDNA cleavage activity (FIG. 27B; FIG. 37A), similar to the mismatch tolerance pattern observed in Cas12a off-target studies. Together, these data are consistent with PAM-mediated dsDNA target binding and the role of base pairing between the crRNA and the target strand to activate trans-ssDNA cutting.

FIGS. 27A-27C. Specificity and conservation of trans-cleavage activation. FIG. 27A LbCas12a-crRNA in the absence or presence of indicated activator, incubated with a radiolabeled non-specific ssDNA substrate (S) for 30 min at 37° C.; products (P) resolved by denaturing PAGE. FIG. 27B Observed trans-cleavage rates for LbCas12a using a ssDNA or dsDNA activator with indicated mismatches; rates represent the average of three different targets measured in triplicate, and error bars represent mean±s.d., where n=9 (three replicates for three independent targets). FIG. 27C Radiolabeled cis (complementary) or trans (non-complementary) substrates were incubated with Cas12a-crRNA or Cas9-sgRNA in the presence or absence of a ssDNA activator for 30 min at 37° C.; a cis-dsDNA substrate was used in the “no enzyme” lanes. Substrate (S) and nucleotide products (P) were resolved by denaturing PAGE.

FIG. 37. The PAM sequence and PAM-proximal mismatches in a dsDNA activator provided specificity for trans-activation. Quantification of trans-cleavage kinetics using mismatched substrates for three distinct target sequences; error bars represent the mean±s.d., where n=3 replicates.

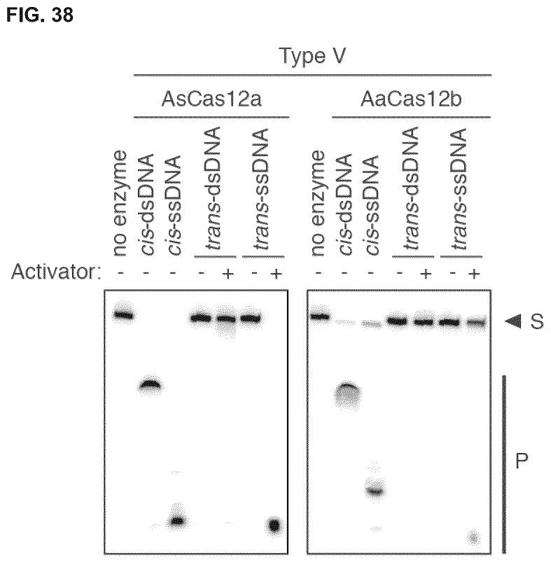

The data suggested that this trans-ssDNA cutting activity might be a property shared by other Cas12a enzymes, and perhaps more evolutionarily distinct type V CRISPR effector proteins, considering that all type V effectors contain a single RuvC nuclease domain. Consistent with this possibility, purified Cas12a orthologs from Acidaminococcus sp. (AsCas12a) and Francisella novicida (FnCas12a), as well as a Cas12b protein from Alicyclobacillus acidoterrestris (AaCas12b), all catalyzed non-specific ssDNase cleavage when assembled with crRNA and a complementary ssDNA activator (FIG. 27C; FIG. 38). In contrast, none of the type II CRISPR-Cas9 proteins tested showed evidence for trans-ssDNA cleavage (FIG. 27C;

FIG. 38), suggesting that target-dependent activation of non-specific ssDNA cleavage is a fundamental feature of all type V CRISPR-Cas12 proteins. These results reveal the unexpected functional convergence of Cas12 enzymes with the type III CRISPR-Csm/Cmr and type VI CRISPR-Cas13 effectors, which also exhibit target-activated, non-specific ssDNase or ssRNase activity, respectively.

FIG. 38. Activator-dependent, non-specific ssDNA cleavage activity was found to be conserved across type V CRISPR interference proteins. Radiolabeled cis (complementary) or trans (non-complementary) substrates were incubated with Cas12-crRNA in the presence or absence of a ssDNA activator for 30 min at 37° C. (or 47.5° C. for AaCas12b). For cis-dsDNA, non-target strand is 5′ end labeled, while the target strand (complementary to guide RNA) is 5′ end labeled for cis-ssDNA; trans-ssDNA and dsDNA are non-specific DNAs. In “no enzyme” lanes, 5′ end labeled trans-ssDNA is loaded. Substrate (S) and nucleotide products (P) are resolved by denaturing PAGE.

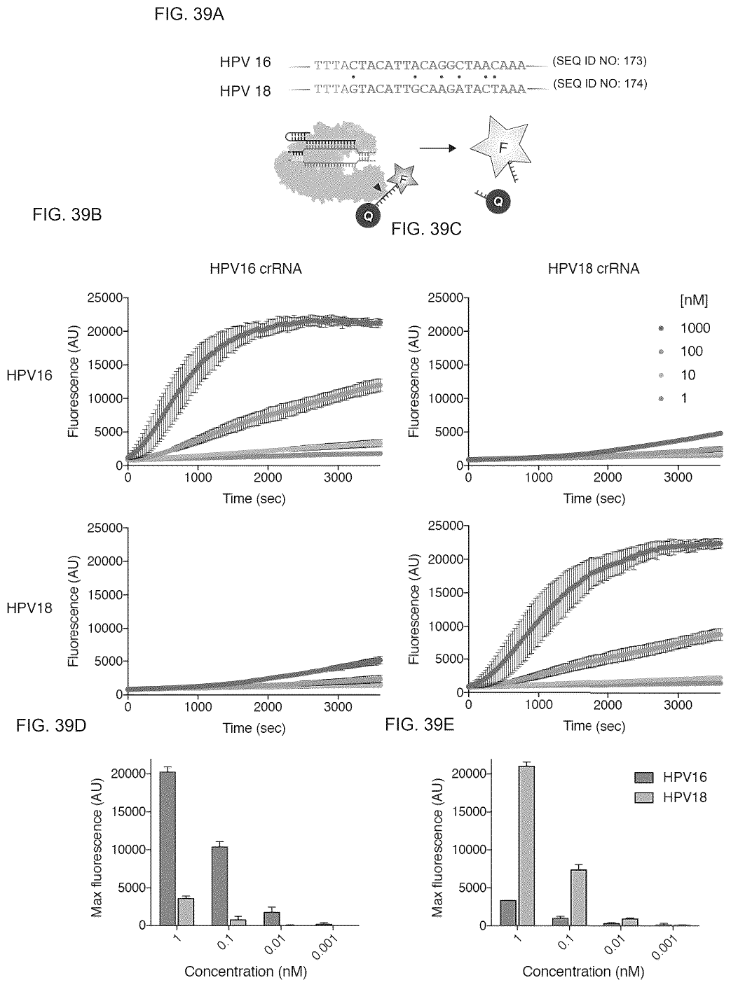

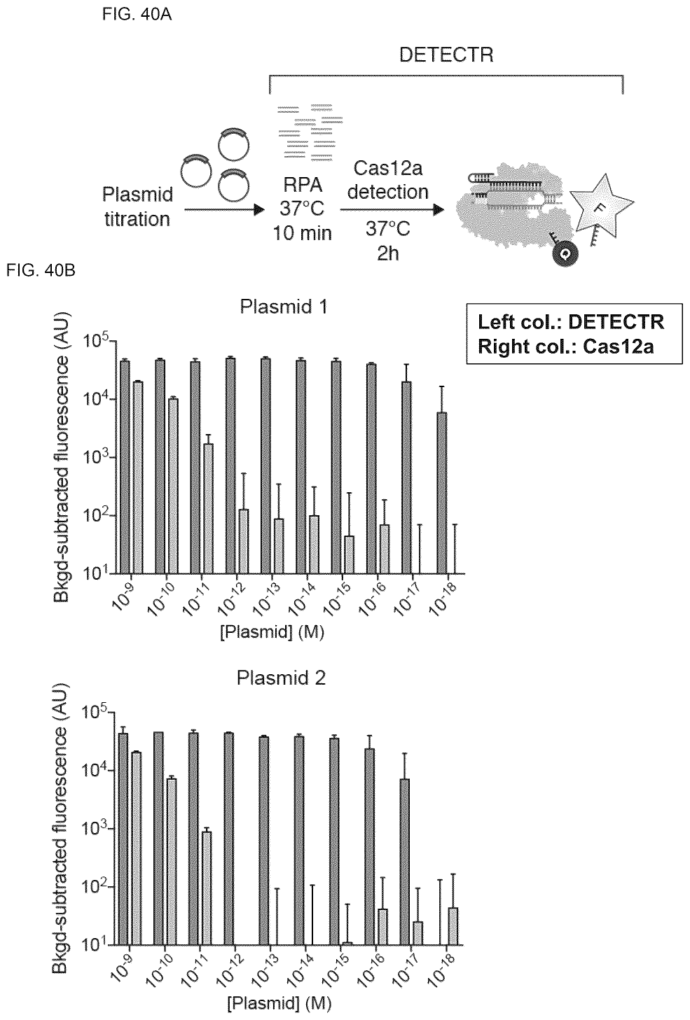

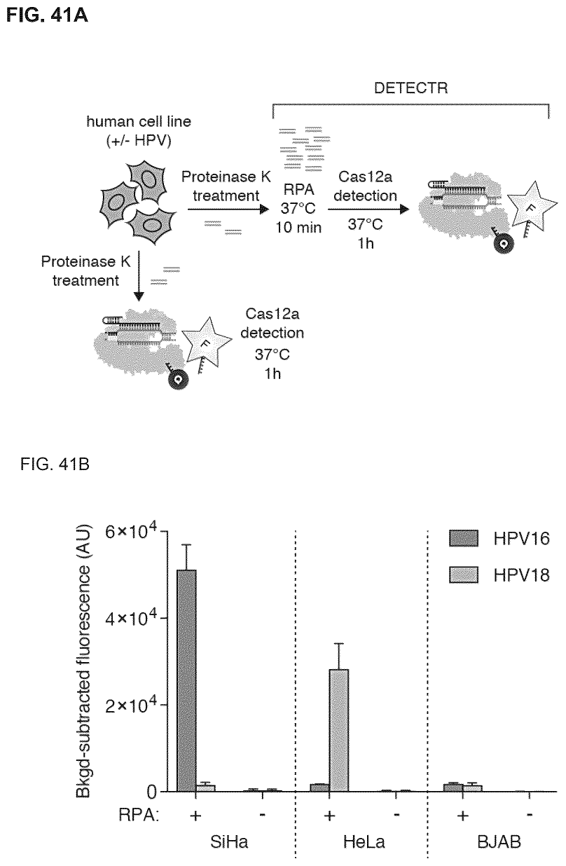

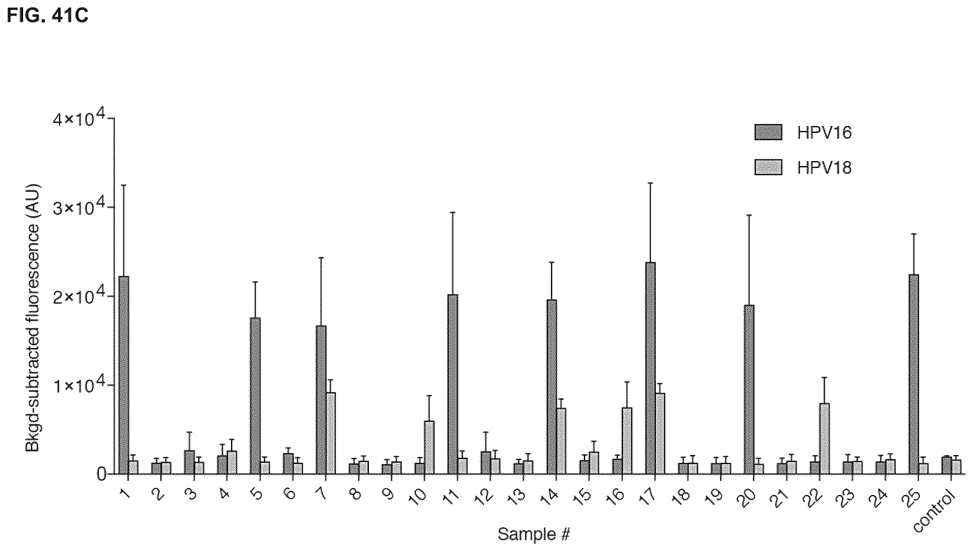

It was next tested whether LbCas12a could be repurposed as a DNA detection platform for use in clinical specimens, based on its ability to induce a fluorescent readout in response to a specific dsDNA sequence. In particular, accurate and rapid identification of human papillomavirus (HPV) is critical for identification of those at risk of HPV-related pre-cancer and cancer, with types 16 (HPV16) and 18 (HPV18) accounting for the majority of precancerous lesions. To test if LbCas12a-catalyzed trans-ssDNA cleavage can distinguish between these two dsDNA viruses, a 20 nt target sequence located next to a TTTA PAM that varied by only six base pairs between the two HPV genotypes was selected (FIGS. 39A-39E). Plasmids containing a ˜500 bp fragment of the HPV16 or HPV18 genome, including the target sequence, were incubated with the LbCas12a-crRNA complex targeting either the HPV16 or HPV18 fragment and a quenched-fluorescent ssDNA reporter. After one hour, LbCas12a produced a robust fluorescent signal only in the presence of the cognate HPV target, whose identity could be distinguished down to ˜10 pM of plasmid (FIGS. 39A-39E). To enhance assay sensitivity, isothermal amplification by Recombinase Polymerase Amplification (RPA) was coupled with LbCas12a to develop a rapid one-pot detection method termed DNA Endonuclease Targeted CRISPR Trans Reporter (DETECTR) (FIG. 40A). When programmed to recognize its cognate plasmid, DETECTR was able to identify targets with attomolar sensitivity (FIG. 40B).

FIGS. 39A-39E. Cas12a distinguishes two closely related HPV sequences. FIG. 39A. Alignment of 20 nt targeting sequences within HPV16 and HPV18 genomes that differ by 6 nucleotides, with a schematic of Cas12a detection using a ssDNA-FQ reporter. Fluorescence timecourses with LbCas12a preassembled with a crRNA targeting FIG. 39B HPV16 or FIG. 39C HPV16 in the presence of a dsDNA plasmid containing an HPV16 (top row) or HPV18 (middle row) genomic fragment and DNaseAlert substrate, with fluorescence measurements taken every 30 seconds for 1 h at 37° C. FIG. 39D. Maximum fluorescence signal obtained from timecourses in FIG. 39B and FIG. 39C. Error bars represent mean±s.d., where n=3 replicates.

FIGS. 40A-40B. Isothermal amplification coupled with Cas12a detection yielded DETECTR, which achieved attomolar sensitivity. FIG. 40A Schematic of DETECTR, consisting of isothermal amplification by RPA and Cas12a detection using a ssDNA-FQ reporter. FIG. 40B Titration of two independent plasmids detected by DETECTR or Cas12a alone. Note that DETECTR achieve attomolar sensitivity. Error bars represent mean±s.d., where n=3 replicates.

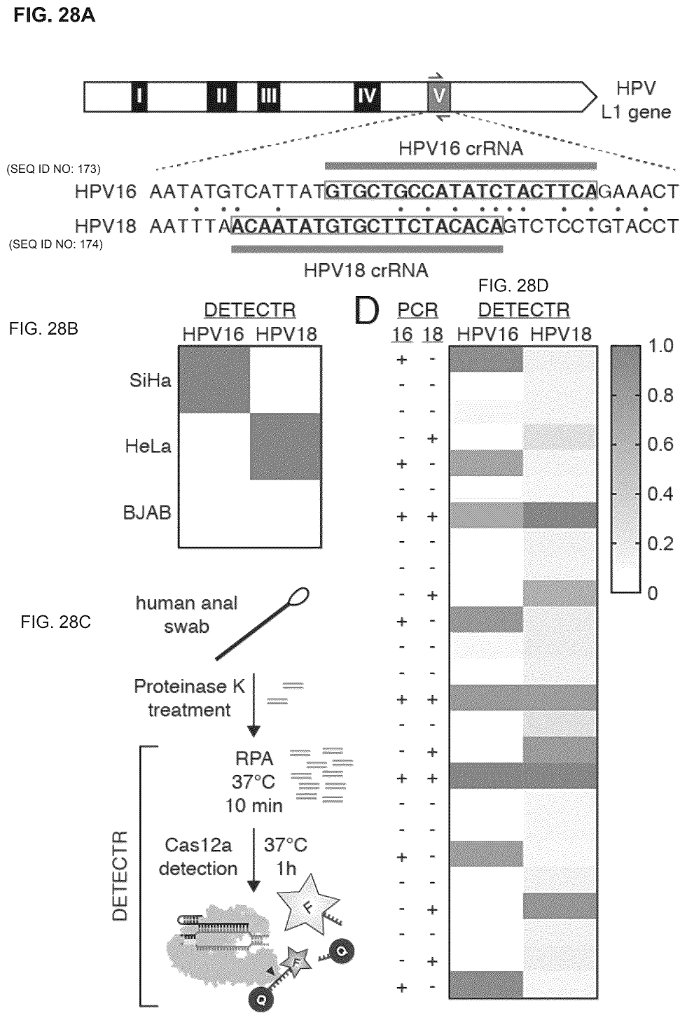

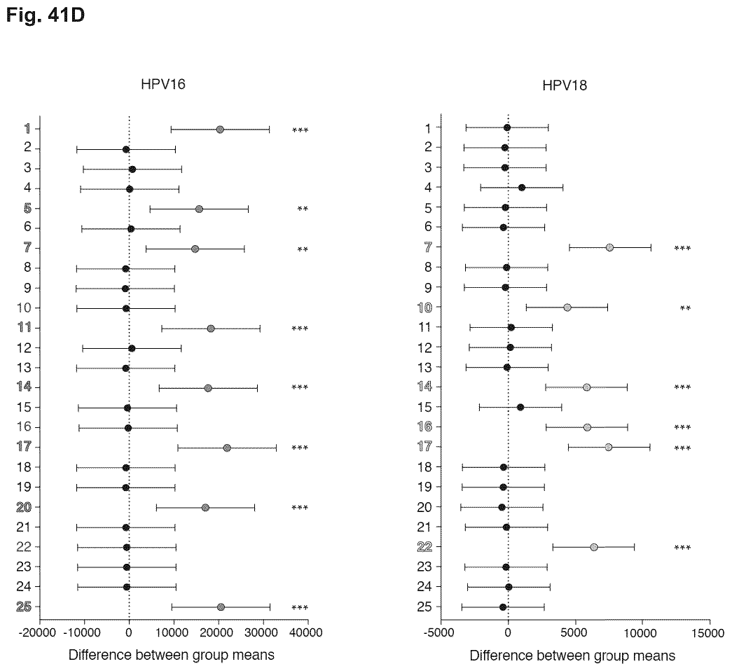

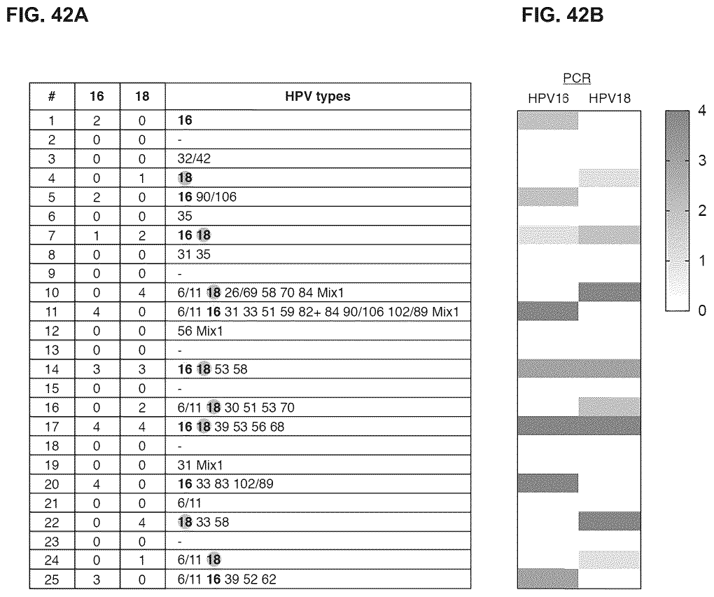

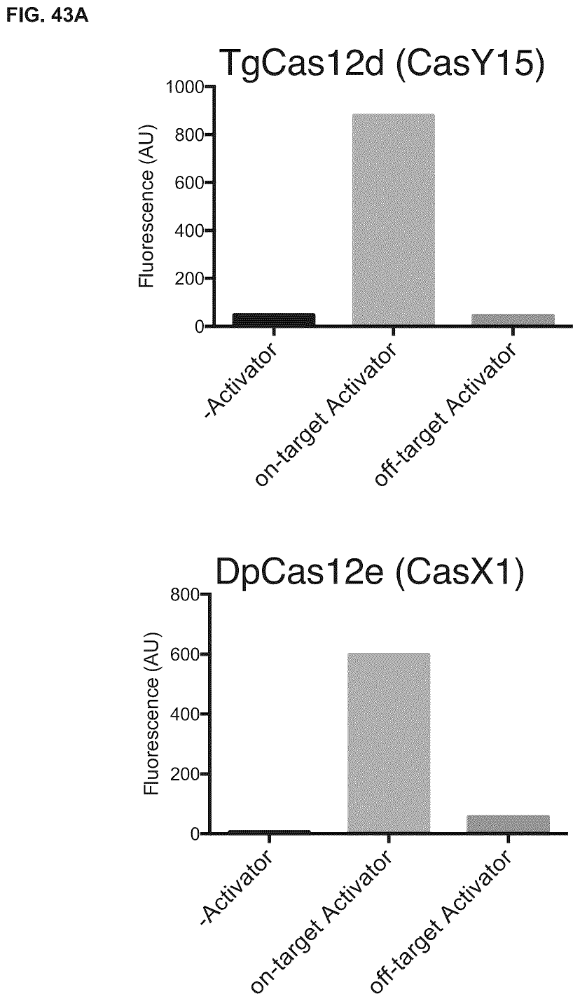

To assess whether HPV could be detected in more complex mixtures, DNA extracted from cultured human cells infected with HPV types 16 (SiHa), 18 (HeLa), or without HPV (BJAB) was added to LbCas12a complexed with a crRNA targeting the hypervariable loop V of the L1 gene within HPV16 or HPV18 (FIG. 28A).

Whereas LbCas12a-crRNA alone was not sensitive enough to detect HPV, DETECTR unambiguously identified HPV types 16 and 18 only in SiHa and HeLa cells, respectively (FIG. 28B; FIGS. 41A-41B). To investigate the utility of DETECTR on patient samples, crude DNA extractions from 25 human anal swabs previously analyzed by a PCR-based method for HPV infection were tested (FIGS. 42A-42B). Within one hour, DETECTR accurately identified the presence or absence of HPV16 (25/25 agreement) and HPV18 (23/25 agreement) in 25 patient samples containing a heterogeneous mixture of HPV types, with good correlation between the PCR-based intensity and DETECTR signal (FIGS. 28C-28D; FIGS. 41C-41D; FIGS. 42A-42B). Furthermore, the absence of fluorescence signal in specimens that were not infected with HPV types 16 or 18, but did contain other HPV types, was an indicator of good specificity by DETECTR. These results demonstrate a new platform for CRISPR-based diagnostics, and suggest that DETECTR could in principle be extended to rapidly detect any DNA sequence of interest with high sensitivity and specificity.

FIGS. 28A-28D. Rapid identification of HPV types 16 and 18 in human samples by DETECTR. FIG. 28A. Diagram of HPV16 and HPV18 sequences within the hypervariable loop V of the L1 gene targeted by Cas12a; highlighted bases indicate 5′ PAM sequence. FIG. 28B. Heatmap represents normalized mean fluorescence values of HPV types 16 and 18 detected in human cell lines by DETECTR; normalized scale represented in FIG. 28D. FIG. 28C. Schematic outlining DNA extraction from human anal samples to HPV identification by DETECTR. FIG. 28D. Identification of HPV types 16 and 18 in 25 patient samples by PCR (left) and DETECTR (right); DETECTR heatmap represents normalized mean fluorescence values.

FIGS. 41A-41D. Identification of HPV types 16 and 18 in human cell lines and patient samples by DETECTR. FIG. 41A Schematic of HPV detection by DETECTR or Cas12a alone. FIG. 41B Detection of HPV types 16 or 18 in SiHa (integrated HPV16), HeLa (integrated HPV18) and BJAB (no HPV) human cell lines, with or without RPA amplification. FIG. 41C. Detection of HPV types 16 or 18 by DETECTR in 25 human anal clinical samples; BJAB cell line (no HPV) used as a control. Error bars represent mean±s.d., where n=3 replicates. FIG. 41D Plot of 95% confidence intervals of difference between control and sample groups, based on a one-way ANOVA with Dunnett's post test, where n=3 replicates. Highlighted sample numbers indicate positive detection of HPV16 (left) or HPV18 (right) in patient samples, where **p≤0.01 and ***p≤0.001.

FIGS. 42A-42B. PCR and hybrid capture validation and genotyping of HPV in human clinical samples. FIG. 42A Summary of PCR-based detection of HPV types 16 (column 2 and yellow circles) and 18 (column 3 and orange circles) and identification of other HPV types by PCR in 25 in patient samples (column 4) (2); subjective intensive values (0-4 scale) were assigned for each PCR-based validation (columns 2 and 3). FIG. 42B Heatmap depiction of PCR results.

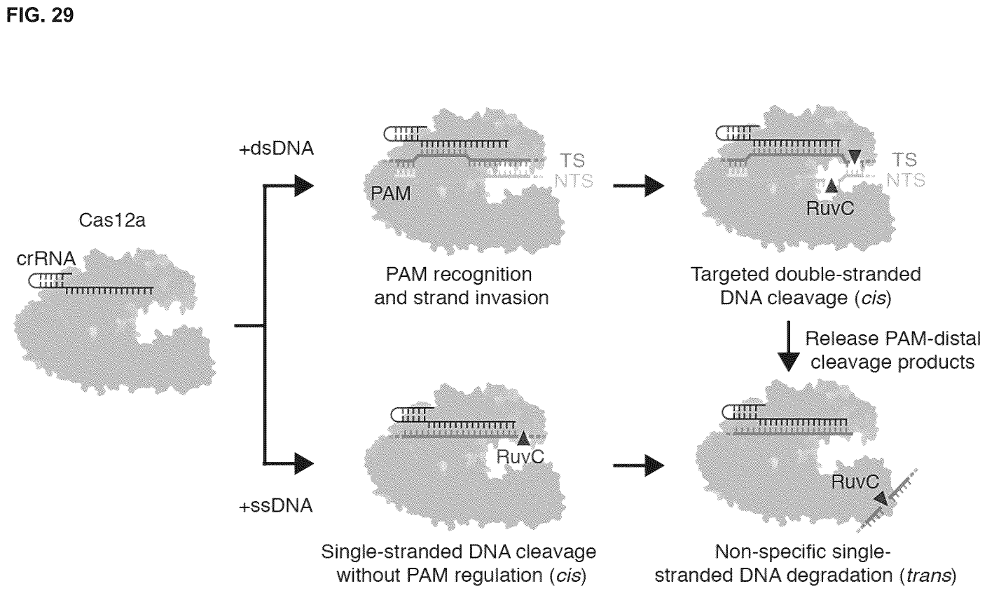

Together, these findings support a unifying mechanism of target interference that begins with the Cas12-guide RNA complex binding to a complementary DNA sequence in a PAM-dependent (dsDNA) or PAM-independent (ssDNA) manner (FIG. 29). Within a host bacterium, such enzyme activation could provide simultaneous protection from both dsDNA and ssDNA phages, and could also target ssDNA sequences that arise temporarily during phage replication or transcription. In a genome-editing context, target-activated ssDNA cutting by Cas12 may be a rare event, but it has the potential to cleave transiently exposed ssDNA at replication forks, R-loops and transcription bubbles, or ssDNA templates used for homology-directed repair. Finally, unleashing the ssDNase activity of Cas12 proteins offers a new strategy to improve the speed, sensitivity and specificity of nucleic acid detection for point-of-care diagnostic applications.

FIG. 29. Model for PAM-dependent and PAM-independent activation of cis and trans-cleavage by Cas12a. The Cas12a-crRNA complex binds to a complementary dsDNA in a PAM-dependent manner (top) or ssDNA in a PAM-independent manner (bottom), which is sufficient to unleash indiscriminate ssDNase activity by the RuvC nuclease. Cas12 proteins (e.g., Cas12a) can also release their PAM-distal cleavage products, which exposes the RuvC active site for multiple rounds of non-specific ssDNA degradation.

TABLE 2
Nucleic acids used in this study.

| | | |
|---|---|---|
| | | SEQ |
| Name | Sequence | ID NO. |

RNA

| | | |
|---|---|---|
| LbCas12a crRNA - Target 1 | UAAUUUCUACUAAGUGUAGAUCGUC | 20 |
| | GCCGUCCAGCUCGACC | |
| LbCas12a crRNA - pUC19 | UAAUUUCUACUAAGUGUAGAUCAAC | 21 |
| | GUCGUGACUGGGAAAACCCU | |
| LbCas12a crRNA - M13 | UAAUUUCUACUAAGUGUAGAUAACG | 22 |
| | AACCACCAGCAGAAGA | |
| LbCas12a crRNA - Target 2 | UAAUUUCUACUAAGUGUAGAUGAUC | 23 |
| | GUUACGCUAACUAUGA | |
| LbCas12a crRNA - Target 3 | UAAUUUCUACUAAGUGUAGAUCCUG | 24 |
| | GGUGUUCCACAGCUGA | |
| LbCas12a crRNA - Plasmid 1 | UAAUUUCUACUAAGUGUAGAUCUAC | 25 |
| | AUUACAGGCUAACAAA | |
| LbCas12a crRNA - Plasmid 2 | UAAUUUCUACUAAGUGUAGAUGUAC | 26 |
| | AUUGCAAGAUACUAAA | |
| LbCas12a crRNA - HPV16-L1 | UAAUUUCUACUAAGUGUAGAUUGAA | 27 |
| | GUAGAUAUGGCAGCAC | |
| LbCas12a crRNA - HPV18-L1 | UAAUUUCUACUAAGUGUAGAUACAA | 28 |
| | UAUGUGCUUCUACACA | |
| AsCas12a crRNA - Target 2 | UAAUUUCUACUCUUGUAGAUGAUCG | 29 |
| | UUACGCUAACUAUGA | |
| FnCas12a crRNA - Target 2 | UAAUUUCUACUGUUGUAGAUGAUCG | 30 |
| | UUACGCUAACUAUGA | |
| AaCas12b crRNA - Target 2 | GUCUAGAGGACAGAAUUUUUCAACGG | 31 |
| | GUGUGCCAAUGGCCACUUUCCAGGUG | |
| | GCAAAGCCCGUUGAGCUUCUCAAAUC | |
| | UGAGAAGUGGCACGAUCGUUACGCU | |
| | AACUAUGA | |
| SpCas9 sgRNA - Target 1 | CGUCGCCGUCCAGCUCGACCGUUUU | 32 |
| | AGAGCUAUGCUGUUUUGGAAACAAAA | |
| | CAGCAUAGCAAGUUAAAAUAAGGCUA | |
| | GUCCGUUAUCAACUUGAAAAAGUGGC | |
| | ACCGAGUCGGUGC | |
| SpCas9 sgRNA - M13 | AACGAACCACCAGCAGAAGAGUUUU | 33 |
| | AGAGCUAUGCUGUUUUGGAAACAAAA | |
| | CAGCAUAGCAAGUUAAAAUAAGGCUA | |
| | GUCCGUUAUCAACUUGAAAAAGUGGC | |
| | ACCGAGUCGGUGC | |
| SpCas9 sgRNA - Target 2 | GAUCGUUACGCUAACUAUGAGUUUU | 34 |
| | AGAGCUAUGCUGUUUUGGAAACAAAA | |
| | CAGCAUAGCAAGUUAAAAUAAGGCUA | |
| | GUCCGUUAUCAACUUGAAAAAGUGGC | |
| | ACCGAGUCGGUGC | |
| NmCas9 sgRNA - Target 2 | GAUCGUUACGCUAACUAUGAGUUGU | 35 |
| | AGCUCCCUUUCUCAUUUCGCAGUGCG | |
| | AAAGCACUGCGAAAUGAGAACCGUUG | |
| | CUACAAUAAGGCCGUCUGAAAAGAUG | |
| | UGCCGCAACGCUCUGCCCCUUAAAGC | |
| | UUCUGC | |
| CjCas9 sgRNA - Target 2 | GAUCGUUACGCUAACUAUGAGUUUU | 36 |
| | AGUCCCUUUUUAAAUUUCUUUAUGGU | |
| | AAAAUUAUAAUCUCAUAAGAAAUUU | |
| | AAAAAGGGACUAAAAUAAAGAGUUU | |
| | GCGGGACUCUGCGGGGUUACAAUCCC | |
| | CUAAAACCGCUU | |
| Target 1 ssRNA | GCCGGGGUGGUGCCCAUCCUGGUCGA | 37 |
| | GCUGGACGGCGACGUAAACGGCCACA | |
| | AGC | |
| Target 2 ssRNA | UAGCAUUCCACAGACAGCCCUCAUAG | 38 |
| | UUAGCGUAACGAUCUAAAGUUUUGUC | |
| | GUC | |

DNA

| | | |
|---|---|---|
| non-specific NTS | AGCTTGTCTGCCATGGACATGCAGACT | 39 |
| | ATACTGTTATTGTTGTACAGACCGAAT | |
| | TCCC | |
| non-specific TS | GGGAATTCGGTCTGTACAACAATAACA | 40 |
| | GTATAGTCTGCATGTCCATGGCAGACA | |
| | AGCT | |
| Target 1_NTS | GCTTGTGGCCGTTTACGTCGCCGTCC | 41 |
| | AGCTCGACCAGGATGGGCACCACCCC | |
| | GGC | |
| Target 1_TS | GCCGGGGTGGTGCCCATCCTGGTCGA | 42 |
| | GCTGGACGGCGACGTAAACGGCCAC | |
| | AAGC | |
| Target 1_20-19_NTS | GCTTGTGGCCGTTTACGTCGCCGTCC | 43 |
| | AGCTCGAGGAGGATGGGCACCACCCC | |
| | GGC | |
| Target 1_20-19_TS | GCCGGGGTGGTGCCCATCCTCCTCGA | 44 |
| | GCTGGACGGCGACGTAAACGGCCAC | |
| | AAGC | |
| Target 1_18-17_NTS | GCTTGTGGCCGTTTACGTCGCCGTCC | 45 |
| | AGCTCCTCCAGGATGGGCACCACCCC | |
| | GGC | |
| Target 1_18-17_TS | GCCGGGGTGGTGCCCATCCTGGAGGA | 46 |
| | GCTGGACGGCGACGTAAACGGCCAC | |
| | AAGC | |
| Target 1_16-15_NTS | GCTTGTGGCCGTTTACGTCGCCGTCC | 47 |
| | AGCAGGACCAGGATGGGCACCACCCC | |
| | GGC | |
| Target 1_16-15_TS | GCCGGGGTGGTGCCCATCCTGGTCCT | 48 |
| | GCTGGACGGCGACGTAAACGGCCAC | |
| | AAGC | |
| Target 1_14-13_NTS | GCTTGTGGCCGTTTACGTCGCCGTCC | 49 |
| | ACGTCGACCAGGATGGGCACCACCCC | |
| | GGC | |
| Target 1_14-13_TS | GCCGGGGTGGTGCCCATCCTGGTCGA | 50 |
| | CGTGGACGGCGACGTAAACGGCCAC | |
| | AAGC | |
| Target 1_12-11_NTS | GCTTGTGGCCGTTTACGTCGCCGTCG | 51 |
| | TGCTCGACCAGGATGGGCACCACCCC | |
| | GGC | |
| Target 1_12-11_TS | GCCGGGGTGGTGCCCATCCTGGTCGA | 52 |
| | GCACGACGGCGACGTAAACGGCCAC | |
| | AAGC | |
| Target 1_10-9_NTS | GCTTGTGGCCGTTTACGTCGCCGAGC | 53 |
| | AGCTCGACCAGGATGGGCACCACCCC | |
| | GGC | |
| Target 1_10-9_TS | GCCGGGGTGGTGCCCATCCTGGTCGA | 54 |
| | GCTGCTCGGCGACGTAAACGGCCAC | |
| | AAGC | |
| Target 1_8-7_NTS | GCTTGTGGCCGTTTACGTCGCGCTCC | 55 |
| | AGCTCGACCAGGATGGGCACCACCCC | |
| | GGC | |
| Target 1_8-7_TS | GCCGGGGTGGTGCCCATCCTGGTCGA | 56 |
| | GCTGGAGCGCGACGTAAACGGCCAC | |
| | AAGC | |
| Target 1_6-5_NTS | GCTTGTGGCCGTTTACGTCCGCGTCC | 57 |
| | AGCTCGACCAGGATGGGCACCACCCC | |
| | GGC | |
| Target 1_6-5_TS | GCCGGGGTGGTGCCCATCCTGGTCGA | 58 |
| | GCTGGACGCGGACGTAAACGGCCAC | |
| | AAGC | |
| Target 1_4-3_NTS | GCTTGTGGCCGTTTACGAGGCCGTCC | 59 |
| | AGCTCGACCAGGATGGGCACCACCCC | |
| | GGC | |
| Target 1_4-3_TS | GCCGGGGTGGTGCCCATCCTGGTCGA | 60 |
| | GCTGGACGGCCTCGTAAACGGCCAC | |
| | AAGC | |
| Target 1_2-1_NTS | GCTTGTGGCCGTTTAGCTCGCCGTCC | 61 |
| | AGCTCGACCAGGATGGGCACCACCCC | |
| | GGC | |
| Target 1_2-1_TS | GCCGGGGTGGTGCCCATCCTGGTCGA | 62 |
| | GCTGGACGGCGAGCTAAACGGCCAC | |
| | AAGC | |
| Target 1_mut-PAM_NTS | GCTTGTGGCCGAGCACGTCGCCGTCC | 63 |
| | AGCTCGACCAGGATGGGCACCACCCC | |
| | GGC | |
| Target 1_mut-PAM_TS | GCCGGGGTGGTGCCCATCCTGGTCGA | 64 |
| | GCTGGACGGCGACGTGCTCGGCCACA | |
| | AGC | |
| Target 1_5nt_TS | CGACGTAAACGGCCACAAGC | 65 |
| Target 1_10nt_TS | GACGGCGACGTAAACGGCCACAAGC | 66 |
| Target 1_15nt_TS | AGCTGGACGGCGACGTAAACGGCCA | 67 |
| | CAAGC | |
| Target 1_20nt_TS | GGTCGAGCTGGACGGCGACGTAAAC | 68 |
| | GGCCACAAGC | |
| Target 1_25nt_TS | ATCCTGGTCGAGCTGGACGGCGACG | 69 |
| | TAAACGGCCACAAGC | |
| Target 1_5nt_NTS | GCTTGTGGCCGTTTACGTCG | 70 |
| Target 1_10nt_NTS | GCTTGTGGCCGTTTACGTCGCCGTC | 71 |
| Target 1_15nt_NTS | GCTTGTGGCCGTTTACGTCGCCGTCC | 72 |
| | AGCT | |
| Target 1_20nt_NTS | GCTTGTGGCCGTTTACGTCGCCGTCC | 73 |
| | AGCTCGACC | |
| Target 1_25nt_NTS | GCTTGTGGCCGTTTACGTCGCCGTCC | 74 |
| | AGCTCGACCAGGAT | |
| Target 2_NTS | GACGACAAAACTTTAGATCGTTACGC | 75 |
| | TAACTATGAGGGCTGTCTGTGGAATG | |
| | CTA | |
| Target 2_TS | TAGCATTCCACAGACAGCCCTCATAGT | 76 |
| | TAGCGTAACGATCTAAAGTTTTGTCG | |
| | TC | |
| Target 2_20-19_NTS | GACGACAAAACTTTAGATCGTTACGC | 77 |
| | TAACTATCTGGGCTGTCTGTGGAATGC | |
| | TA | |
| Target 2_20-19_TS | TAGCATTCCACAGACAGCCCAGATAG | 78 |
| | TTAGCGTAACGATCTAAAGTTTTGTC | |
| | GTC | |
| Target 2_18-17_NTS | GACGACAAAACTTTAGATCGTTACGC | 79 |
| | TAACTTAGAGGGCTGTCTGTGGAATG | |
| | CTA | |
| Target 2_18-17_TS | TAGCATTCCACAGACAGCCCTCTAAGT | 80 |
| | TAGCGTAACGATCTAAAGTTTTGTCG | |
| | TC | |
| Target 2_16-15_NTS | GACGACAAAACTTTAGATCGTTACGC | 81 |
| | TAAGAATGAGGGCTGTCTGTGGAATG | |
| | CTA | |
| Target 2_16-15_TS | TAGCATTCCACAGACAGCCCTCATTCT | 82 |
| | TAGCGTAACGATCTAAAGTTTTGTCG | |
| | TC | |
| Target 2_14-13_NTS | GACGACAAAACTTTAGATCGTTACGC | 83 |
| | TTTCTATGAGGGCTGTCTGTGGAATGC | |
| | TA | |
| Target 2_14-13_TS | TAGCATTCCACAGACAGCCCTCATAG | 84 |
| | AAAGCGTAACGATCTAAAGTTTTGTC | |
| | GTC | |
| Target 2_12-11_NTS | GACGACAAAACTTTAGATCGTTACGG | 85 |
| | AAACTATGAGGGCTGTCTGTGGAATG | |
| | CTA | |
| Target 2_12-11_TS | TAGCATTCCACAGACAGCCCTCATAGT | 86 |
| | TTCCGTAACGATCTAAAGTTTTGTCGT | |
| | C | |
| Target 2_10-9_NTS | GACGACAAAACTTTAGATCGTTAGCC | 87 |
| | TAACTATGAGGGCTGTCTGTGGAATG | |
| | CTA | |
| Target 2_10-9_TS | TAGCATTCCACAGACAGCCCTCATAGT | 88 |
| | TAGGCTAACGATCTAAAGTTTTGTCG | |
| | TC | |
| Target 2_8-7_NTS | GACGACAAAACTTTAGATCGTATCGC | 89 |
| | TAACTATGAGGGCTGTCTGTGGAATG | |
| | CTA | |
| Target 2_8-7_TS | TAGCATTCCACAGACAGCCCTCATAGT | 90 |
| | TAGCGATACGATCTAAAGTTTTGTCG | |
| | TC | |
| Target 2_6-5_NTS | GACGACAAAACTTTAGATCCATACGC | 91 |
| | TAACTATGAGGGCTGTCTGTGGAATG | |
| | CTA | |
| Target 2_6-5_TS | TAGCATTCCACAGACAGCCCTCATAGT | 92 |
| | TAGCGTATGGATCTAAAGTTTTGTCG | |
| | TC | |
| Target 2_4-3_NTS | GACGACAAAACTTTAGAAGGTTACGC | 93 |
| | TAACTATGAGGGCTGTCTGTGGAATG | |
| | CTA | |
| Target 2_4-3_TS | TAGCATTCCACAGACAGCCCTCATAGT | 94 |
| | TAGCGTAACCTTCTAAAGTTTTGTCGT | |
| | C | |
| Target 2_2-1_NTS | GACGACAAAACTTTACTTCGTTACGC | 95 |
| | TAACTATGAGGGCTGTCTGTGGAATG | |
| | CTA | |
| Target 2_2-1_TS | TAGCATTCCACAGACAGCCCTCATAGT | 96 |
| | TAGCGTAACGAAGTAAAGTTTTGTCG | |
| | TC | |
| Target 2_mut-PAM_NTS | GACGACAAAACAGCAGATCGTTACGC | 97 |
| | TAACTATGAGGGCTGTCTGTGGAATG | |
| | CTA | |
| Target 2_mut-PAM_TS | TAGCATTCCACAGACAGCCCTCATAGT | 98 |
| | TAGCGTAACGATCTGCTGTTTTGTCGT | |
| | C | |
| Target 2_NmCas9_NTS | GACGACAAAACTTTAGATCGTTACGC | 99 |
| | TAACTATGAGGGCGAGTTGTGGAATG | |
| | CTA | |
| Target 2_NmCas9_TS | TAGCATTCCACAACTCGCCCTCATAGT | 100 |
| | TAGCGTAACGATCTAAAGTTTTGTCG | |
| | TC | |
| Target 2_CjCas9_TS | GACGACAAAACTTTAGATCGTTACGC | 101 |
| | TAACTATGAGGGCCAAATGTGGAATG | |
| | CTA | |
| Target 2_CjCas9_TS | TAGCATTCCACATTTGGCCCTCATAGT | 102 |
| | TAGCGTAACGATCTAAAGTTTTGTCG | |
| | TC | |
| Target 3_NTS | AGTTGTGTTAGTTTACCTGGGTGTTCC | 103 |
| | ACAGCTGATAGTGATTGCCTTGAATA | |
| | AA | |
| Target 3_TS | TTTATTCAAGGCAATCACTATCAGCTG | 104 |
| | TGGAACACCCAGGTAAACTAACACAA | |
| | CT | |
| Target 3_20-19_NTS | AGTTGTGTTAGTTTACCTGGGTGTTCC | 105 |
| | ACAGCTCTTAGTGATTGCCTTGAATAA | |
| | A | |
| Target 3_20-19_TS | TTTATTCAAGGCAATCACTAAGAGCTG | 106 |
| | TGGAACACCCAGGTAAACTAACACAA | |
| | CT | |
| Target 3_18-17_NTS | AGTTGTGTTAGTTTACCTGGGTGTTCC | 107 |
| | ACAGGAGATAGTGATTGCCTTGAATA | |
| | AA | |
| Target 3_18-17_TS | TTTATTCAAGGCAATCACTATCTCCTG | 108 |
| | TGGAACACCCAGGTAAACTAACACAA | |
| | CT | |
| Target 3_16-15_NTS | AGTTGTGTTAGTTTACCTGGGTGTTCC | 109 |
| | ACTCCTGATAGTGATTGCCTTGAATAA | |
| | A | |
| Target 3_16-15_TS | TTTATTCAAGGCAATCACTATCAGGAG | 110 |
| | TGGAACACCCAGGTAAACTAACACAA | |
| | CT | |
| Target 3_14-13_NTS | AGTTGTGTTAGTTTACCTGGGTGTTCC | 111 |
| | TGAGCTGATAGTGATTGCCTTGAATA | |
| | AA | |
| Target 3_14-13_TS | TTTATTCAAGGCAATCACTATCAGCTC | 112 |
| | AGGAACACCCAGGTAAACTAACACAA | |
| | CT | |
| Target 3_12-11_NTS | AGTTGTGTTAGTTTACCTGGGTGTTG | 113 |
| | GACAGCTGATAGTGATTGCCTTGAAT | |
| | AAA | |
| Target 3_12-11_TS | TTTATTCAAGGCAATCACTATCAGCTG | 114 |
| | TCCAACACCCAGGTAAACTAACACAA | |
| | CT | |
| Target 3_10-9_NTS | AGTTGTGTTAGTTTACCTGGGTGAAC | 115 |
| | CACAGCTGATAGTGATTGCCTTGAAT | |
| | AAA | |
| Target 3_10-9_TS | TTTATTCAAGGCAATCACTATCAGCTG | 116 |
| | TGGTTCACCCAGGTAAACTAACACAA | |
| | CT | |
| Target 3_8-7_NTS | AGTTGTGTTAGTTTACCTGGGACTTCC | 117 |
| | ACAGCTGATAGTGATTGCCTTGAATA | |
| | AA | |
| Target 3_8-7_TS | TTTATTCAAGGCAATCACTATCAGCTG | 118 |
| | TGGAAGTCCCAGGTAAACTAACACAA | |
| | CT | |
| Target 3_6-5_NTS | AGTTGTGTTAGTTTACCTGCCTGTTCC | 119 |
| | ACAGCTGATAGTGATTGCCTTGAATA | |
| | AA | |
| Target 3_6-5_TS | TTTATTCAAGGCAATCACTATCAGCTG | 120 |
| | TGGAACAGGCAGGTAAACTAACACA | |
| | ACT | |
| Target 3_4-3_NTS | AGTTGTGTTAGTTTACCACGGTGTTCC | 121 |
| | ACAGCTGATAGTGATTGCCTTGAATA | |
| | AA | |
| Target 3_4-3_TS | TTTATTCAAGGCAATCACTATCAGCTG | 122 |
| | TGGAACACCGTGGTAAACTAACACAA | |
| | CT | |
| Target 3_2-1_NTS | AGTTGTGTTAGTTTAGGTGGGTGTTC | 123 |
| | CACAGCTGATAGTGATTGCCTTGAAT | |
| | AAA | |
| Target 3_2-1_TS | TTTATTCAAGGCAATCACTATCAGCTG | 124 |
| | TGGAACACCCACCTAAACTAACACAA | |
| | CT | |
| Target 3_mut-PAM_NTS | AGTTGTGTTAGAGCACCTGGGTGTTC | 125 |
| | CACAGCTGATAGTGATTGCCTTGAAT | |
| | AAA | |
| Target 3_mut-PAM_TS | TTTATTCAAGGCAATCACTATCAGCTG | 126 |
| | TGGAACACCCAGGTGCTCTAACACAA | |
| | CT | |

FQ substrates

| | |
|---|---|
| ssDNA-FQ reporter | /56-FAM/TTATT/3IABkFQ/ |
| ssRNA-FQ reporter | /56-FAM/rUrUrArUrU/3IABkFQ/ |
| Dnase-Alert substrate (IDT) | proprietary |

RPA primers

| | | |
|---|---|---|
| Plasmid 1_F | GCAAACCACCTATAGGGGAACAC | 127 |
| Plasmid 1_R | CAGCCAACTCAGCTTCCTTTC | 128 |
| Plasmid 2_F | CATGCCGCCACGTCTAATGTTTC | 129 |
| Plasmid 2_R | GGTGAAGCACGCATACCTGTG | 130 |
| HPV16-L1_F | TTGTTGGGGTAACCAACTATTTGTTAC | 131 |
| | TGTT | |
| HPV16-L1_R | CCTCCCCATGTCTGAGGTACTCCTTAA | 132 |
| | AG | |
| HPV18-L1_F | GCATAATCAATTATTTGTTACTGTGGT | 133 |
| | AGATACCACT | |
| HPV18-L1-R | GCTATACTGCTTAAATTTGGTAGCATC | 134 |
| | ATATTGC | |

### Materials and Methods

Protein expression and purification. DNA sequences encoding SpCas9 and Cas12 proteins and mutants were cloned into a custom pET-based expression vector containing an N-terminal 10×His-tag, maltose-binding protein (MBP) and TEV protease cleavage site. Point mutations were introduced by around-the-horn PCR and verified by DNA sequencing. Proteins were purified as described, with the following modifications: E. coli BL21(DE3) containing SpCas9 or Cas12 expression plasmids were grown in Terrific Broth at 16° C. for 14 hr. Cells were harvested and resuspended in Lysis Buffer (50 mM Tris-HCl, pH 7.5, 500 mM NaCl, 5% (v/v) glycerol, 1 mM TCEP, 0.5 mM PMSF and 0.25 mg/ml lysozyme), disrupted by sonication, and purified using Ni-NTA resin. After overnight TEV cleavage at 4° C., proteins were purified over an MBPTrap HP column connected to a HiTrap Heparin HP column for cation exchange chromatography. The final gel filtration step (Superdex 200) was carried out in elution buffer containing 20 mM Tris-HCl, pH 7.5, 200 mM NaCl (or 250 mM NaCl for AaCas12b), 5% (v/v) glycerol and 1 mM TCEP. All proteins tested in this study are shown in FIG. 30.

Nucleic acid preparation. DNA substrates were synthesized commercially (IDT). For FQ-reporter assays, activator DNA duplexes were prepared by annealing 5-fold molar excess of the NTS to TS in 1× hybridization buffer (20 nM Tris-Cl, pH 7.5, 100 mM KCl, 5 mM MgCl₂), heating at 95° C. and slow-cooling on the benchtop. HPV16 and HPV18 fragments were synthesized as gBlocks (IDT) and cloned into a custom pET-based vector via Gibson assembly. Plasmid DNA for titration experiments was quantified using a Qubit fluorometer (Invitrogen). For radiolabeled cleavage assays, PAGE-purified DNA oligos were prepared as described.

sgRNA templates were PCR amplified from a pUC19 vector or overlapping primers containing a T7 promoter, 20 nucleotide target sequence and an sgRNA scaffold. The amplified PCR product served as the DNA template for in vitro transcription reactions, which were performed as described. crRNAs were transcribed in vitro using a single-stranded DNA template containing a T7 promoter, repeat and spacer in the reverse complement orientation, which was annealed to T7 forward primer in 1× hybridization buffer.

DNA cleavage assays. Generally, Cas12a-mediated cleavage assays were carried out in cleavage buffer consisting of 20 mM HEPES (pH 7.5), 150 mM KCl, 10 mM MgCl₂, 10 glycerol and 0.5 mM DTT. For M13-targeting assays, 30 nM Cas12a was pre-assembled with either 36 nM of M13-targeting crRNA (cis) or with 36 nM of crRNA and 40 nM complementary ssDNA (activator) with no sequence homology to M13 (trans) at 37° C. for 10 min. The reaction was initiated by adding 10 nM M13mp18 ssDNA (New England Biolabs) and incubated at 37° C. for indicated timepoints. Reactions were quenched with DNA loading buffer (30% (v/v) glycerol, 0.25% (w/v) bromophenol blue, 0.25% (w/v) xylene cyanol) containing 15 mM EDTA and separated by 1.5% agarose gel pre-stained with SYBER Gold (Invitrogen).

For radiolabeled cleavage assays, the substrates used were 5′-end-labeled with T4 PNK (NEB) in the presence of gamma ³²P-ATP. For dsDNA substrates, the non-target strand was first 5′-end-labeled and then annealed with excess corresponding target strand. The concentrations of Cas12a (or SpCas9), guide RNA and ³²P-labeled substrates used in the reaction were 30 nM, 36 nM and 1-3 nM (unless otherwise stated), respectively. Reactions were incubated for 30 min (unless otherwise stated) at 37° C. (or 47.5° C. for the thermophilic AacCas12b) and quenched with formamide loading buffer (final concentration 45% formamide and 15 mM EDTA, with trace amount of xylene cyanol and bromophenol blue) for 2-3 min at 90° C. The substrates and products were resolved by 12% urea-denaturing PAGE gel and quantified with Amersham Typhoon (GE Healthcare).

For substrate turnover studies, the pre-assembled Cas12a-crRNA or Cas12a-crRNA-activator (target ssDNA or dsDNA) were incubated at 37° C. for 10 min, and 30 nM of the pre-assembled RNP were used for each reaction with various substrate concentrations at 15, 30, 45, and 60 nM, respectively.

Fluorophore quencher (FQ)-labeled reporter assays. LbCas12a-crRNA complexes were pre-assembled by incubating 200 nM LbCpf1 with 250 nM crRNA and 4 nM activator (ssDNA, dsDNA or ssRNA) at 37° C. for 30 min. The reaction was initiated by diluting LbCas12a complexes to a final concentration of 50 nM LbCas12a:62.5 nM crRNA:1 nM activator in a solution containing 1× Binding Buffer (20 mM Tris-HCl, pH 7.5, 100 mM KCl, 5 mM MgCl₂, 1 mM DTT, 5% glycerol, 50 μg ml⁻¹ heparin) and 50 nM DNaseAlert Substrate™ (IDT) or custom ssDNA/ssRNA FQ reporter substrates in a 20 μl reaction. HPV detection assays were performed as above, with the following modifications: LbCas12a was pre-assembled with an HPV16 or HPV18-targeting crRNA and diluted in a solution containing 1× Binding Buffer, custom ssDNA-FQ reporter and 1, 10, 100, or 1000 nM of HPV16- or HPV18-containing plasmids. Reactions (20 μl, 384-well microplate format) were incubated in a fluorescence plate reader (Tecan Infinite Pro F200) for up to 120 minutes at 37° C. with fluorescence measurements taken every 30 seconds (DNaseAlert substrate=λₑₓ: 535 nm; λₑₘ: 595 nm, custom ssDNA/ssRNA FQ substrates=λₑₓ: 485 nm; λₑₘ: 535 nm).

For trans-cleavage rate determination, background-corrected fluorescence values were calculated by subtracting fluorescence values obtained from reactions carried out in the absence of target plasmid. The resulting data were fit to a single exponential decay curve (GraphPad Software), according to the following equation: Fraction cleaved=A×(1−exp(−k×t)), where A is the amplitude of the curve, k is the first-order rate constant, and t is time.

For Michaelis-Menten analysis, LbCas12a-crRNA-activator (target ssDNA or dsDNA) complexes were prepared as described above, and reaction was initiated by diluting LbCas12a complexes to a final concentration of 5 nM LbCas12a:6.25 nM crRNA:0.1 nM activator (effective complex=0.1 nM) in a solution containing 1× Binding Buffer and 0.001, 0.01, 0.1, 0.2, 0.5, 1 or 2 uM of DNaseAlert™ substrate (IDT). Reactions were incubated in a fluorescence plate reader for up to 30 minutes at 37° C. with fluorescence measurements taken every 30 seconds (λₑₓ: 535 nm; λₑₘ: 595 nm). The initial velocity (V₀) was calculated by fitting to a linear regression and plotted against the substrate concentration to determine the Michaelis-Menten constants (GraphPad Software), according to the following equation: Y=(Vₘₐₓ×X)/(Kₘ+X), where X is the substrate concentration and Y is the enzyme velocity. The turnover number (k_{cat}) was determined by the following equation: k_{cat}=Vₘₐₓ/Eₜ, where Eₜ=0.1 nM.

Human clinical sample collection and DNA preparation. Anal sample donors were recruited from the UCSF Anal Neoplasia Clinic, Research and Education Center (ANCRE). The study was approved by the UCSF Committee on Human Research. After informed consent was obtained, participants had an anal swab inserted into a Thinprep™ vial for anal cytology and HPV testing. Cell suspension left over from the first swab after monolayer cytology slides were made was used for HPV DNA PCR.

A crude DNA preparation was made by pelleting 1.5 ml of the cell suspension. After the pellet was allowed to dry, it was suspended in 100 μl Tris-EDTA with proteinase K (Life Technologies) at a concentration of 200 μg/ml and incubated at 56° C. for 1 hour, then the proteinase K was heat inactivated. Five μl of this was used in the HPV consensus PCR. DNA preparation from human cell lines (SiHa, HeLa, BJAB) was performed as above, with the following modifications: 10⁶-10⁷ cells were harvested, resuspended in 100 μl Tris-EDTA with proteinase K, incubated at 56° C. for 1 hour, then the proteinase K was heat inactivated. One μl of this sample was used for DETECTR experiments.

DETECTR assays. DETECTR combined Recombinase Polymerase Amplification (RPA) using TwistAmp Basic (Twist Biosciences) followed by Cas12a detection in the same reaction. Briefly, 50 μl reactions containing 1 μl sample, 0.48 μM forward and reverse primer, 1× rehydration buffer, 14 mM magnesium acetate and RPA mix were incubated at 37° C. for 10 minutes. The RPA reaction (18 μl) was transferred to a 384-well microplate and 50 nM LbCas12a:62.5 nM crRNA:50 nM custom ssDNA-FQ reporter was added directly to the reaction (20 μl final volume). Reactions were incubated in a fluorescence plate reader (Tecan Infinite Pro F200) for 1-2 h at 37° C. with fluorescence measurements taken every minute (λₑₓ: 485 nm; λₑₘ: 535 nm).

For HPV identification by DETECTR, detection values of HPV types 16 or 18 in human samples were normalized to the maximum mean fluorescence signal obtained using the HPV16- or HPV18-targeting crRNA, respectively. A one-way ANOVA with Dunnett's post test was used to determine the positive cutoff (set at p≤0.05) for identification of HPV16 or HPV18 in patient samples. Based on this cutoff, 100% of samples were accurately identified for HPV16 infection (25/25 agreement with PCR-based results), while 92% of samples were accurately identified for HPV18 infection (23/25 agreement with PCR-based results).

HPV genotyping and validation. PCR was performed as described previously using a modified pool of MY09/MY11 consensus HPV L1 primers as well as primers for amplification of the human beta-globin as an indicator of specimen adequacy as described previously. After 40 amplification cycles, specimens were probed with a biotin-labeled HPV L1 consensus probe mixture. A separate membrane was probed with a biotin-labeled probe to the human beta-globin gene. Specimens were typed by hybridizing to 38 different HPV types, 6/11, 16, 18, 26/69, 30, 31, 32/42, 33, 34, 35, 39, 45, 51, 52, 53, 54, 56, 57/2/27, 58, 59, 61, 62, 66, 67, 68, 70, 71, 72, 73, 81, 82, 83, 84, 85, 86/87, 90/106, 97, 102/89, as well as two separate mixtures. Mix 1 contains 7, 13, 40, 43, 44, 55, 74, and 91, and Mix 2 contains 3, 10, 28, 29, 77, 78, and 94. Specimens negative for beta-globin gene amplification were excluded from analysis. The results of PCR were recorded on a scale from 0 to 5 based on the intensity of the signal on the dot-blots, as described previously. Samples with results recorded as 1 or more were considered to be positive.

### Example 4: Trans-Cleavage Activity of Cas12d and Cas12e

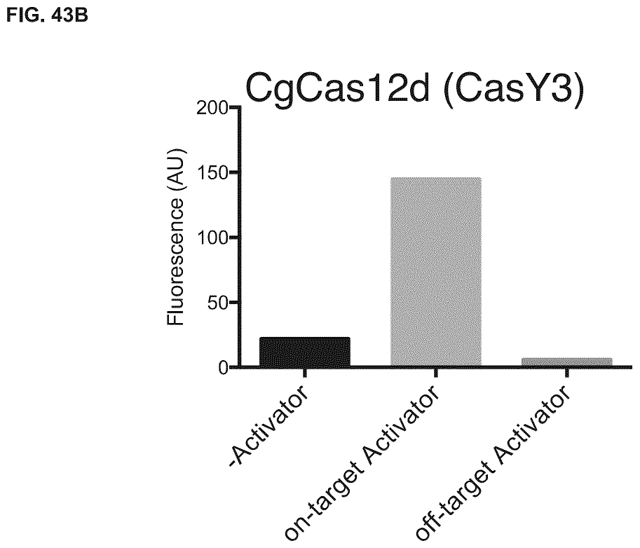

trans-cleavage activity was demonstrated for two additional type V CRISPR/Cas effector proteins, CasX (Cas12e) and CasY (Cas12d) using a DETECTR assay (FIGS. 43A-43B).

### Example 5: Identification of a Single Nucleotide Polymorphism (SNP) within the HERC2 Gene Responsible for Brown or Blue Eyes

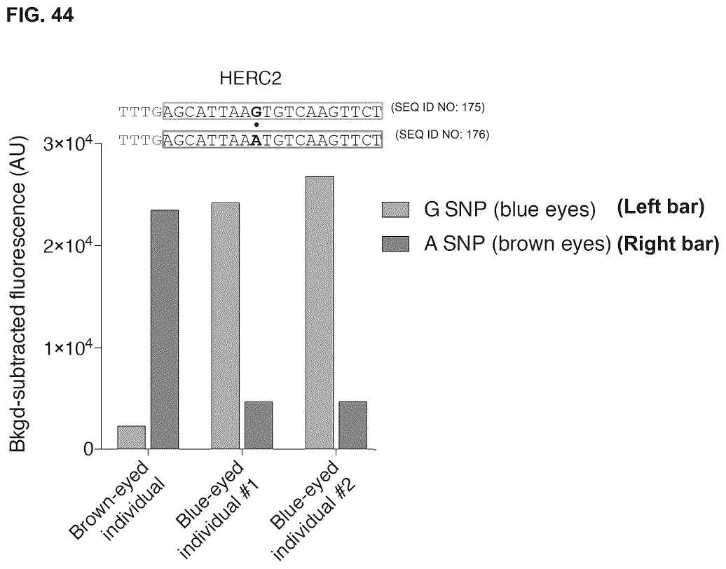

DETECTR was used to detect eye color SNPs from saliva samples using Cas12a (FIG. 44). Sample preparation: 500 μL of phosphate buffered saline was added to ˜500 μL of volunteer saliva and centrifuged for 5 min at 1800 g. The supernatant was decanted and the pellet was resuspended in 100 μL phosphate buffered saline with 0.2% Triton X-100 before incubation at 95° C. for 5 min. 1 μL of sample was used as direct input into RPA reactions. The following nucleic acids were used for these experiments:

RPA primers:
F primer:
(SEQ ID NO: 153)
CAAAGAGAAGCCTCGGCC
R primer:
(SEQ ID NO: 154)
GTGTTAATACAAAGGTACAGGAACAAAGAATTTG
HERC2 G-SNP crRNA:
(SEQ ID NO: 155)
GTAATTTCTACTAAGTGTAGATAGCATTAAGTGTCAAGTTCT
HERC2 A-SNP crRNA:
(SEQ ID NO: 156)
GTAATTTCTACTAAGTGTAGATAGCATTAAATGTCAAGTTCT

### Example 6: Identification of the X or Y Chromosomes Through Detection of the XIST (within X Chromosome) or SRY (within Y Chromosome) Genes from Human Saliva

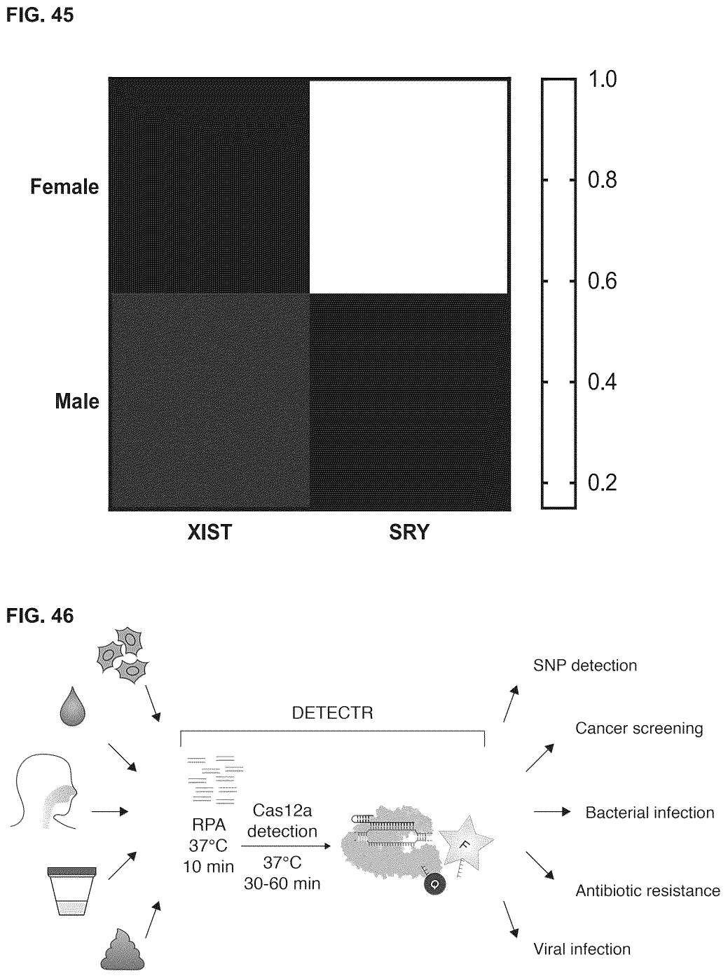

FIG. 45 provides data demonstrating the identification of the X or Y chromosomes through detection of the XIST (within X chromosome) or SRY (within Y chromosome) genes from human saliva. The following nucleic acids were used for these experiments:

XIST crRNA:
(SEQ ID NO: 157)
GTAATTTCTACTAAGTGTAGATACTAGTCCCTTGTACTGATA
SRY crRNA:
(SEQ ID NO: 158)
GTAATTTCTACTAAGTGTAGATGCATTCTGGGATTCTCTAGA
XIST RPA primers:
F primer:
(SEQ ID NO: 159)
CTATCTGAATGAATTGATTTGGGGCTTG
R primer:
(SEQ ID NO: 160)
GCAATGTCAAAATCGCCATTTTAAGC
SRY RPA primers:
F primer:
(SEQ ID NO: 161)
AGGCAACGTCCAGGATAGAGTG
R primer:
(SEQ ID NO: 162)
CAGTAAGCATTTTCCACTGGTATCCCAG

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-45. (canceled)

46. A kit for detecting a target nucleic acid in a sample, comprising:
(i) a type V CRISPR/Cas effector protein, and/or a nucleic acid encoding the type V CRISPR/Cas effector protein;
(ii) one or more of:
(a) a guide RNA, and/or a nucleic acid encoding the guide RNA, wherein the guide RNA comprises a region that binds to the type V CRISPR/Cas effector protein, and a guide sequence that hybridizes with a segment of the target nucleic acid; and
(b) a precursor guide RNA array comprising two or more guide RNAs, each guide RNA comprising a different guide sequence that hybridizes with a segment of the target nucleic acid, and/or a nucleic acid encoding the precursor guide RNA array;
and
(iii) a labeled detector DNA that is single stranded.

47. The kit of claim 46, further comprising a positive control target nucleic acid.

48. The kit of claim 47, further comprising a positive control guide RNA, and/or a nucleic acid encoding the positive control guide RNA, wherein the positive control guide RNA comprises a guide sequence that hybridizes with a segment of the positive control target nucleic acid.

49. The kit of claim 46, further comprising nucleic acid amplification components.

50. The kit of claim 49, wherein the reagents for amplification comprise a primer, a polymerase, or a combination thereof.

51. The kit of claim 46, further comprising reagents for reverse transcription.

52. The kit of claim 51, wherein the reagents for reverse transcription comprise a reverse transcriptase and Rnase H.

53. The kit of claim 46, wherein the target nucleic acid is single stranded or double stranded.

54. The kit of claim 46, wherein the target nucleic acid is DNA or RNA.

55. The kit of claim 46, wherein the target nucleic acid is a viral nucleic acid or a bacterial nucleic acid.

56. The kit of claim 46, wherein the target nucleic acid is a papovavirus, a human papillomavirus (HPV), a hepadnavirus, a Hepatitis B Virus (HBV), a herpesvirus, a varicella zoster virus (VZV), an Epstein Barr virus (EBV), a Kaposi's sarcoma-associated herpesvirus, an adenovirus, a poxvirus, a parvovirus, an influenza virus, or a respiratory syncytial virus nucleic acid.

57. The kit of claim 46, wherein the target nucleic acid is from a human cell.

58. The kit of claim 46, wherein the target nucleic acid is a human fetal nucleic acid or a cancer cell nucleic acid.

59. The kit of claim 46, wherein the type V CRISPR/Cas effector protein is a Cas12 protein.

60. The kit of claim 46, wherein the type V CRISPR/Cas effector protein is a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, or a C2c9 polypeptide.

61. The kit of claim 46, wherein the sample comprises a cell or a cell lysate.

62. The kit of claim 46, wherein the sample is blood, saliva, biopsy, plasma, serum, bronchoalveolar lavage, sputum, a fecal sample, cerebrospinal fluid, a fine needle aspirate, a buccal swab, a cervical swab, a nasal swab, interstitial fluid, synovial fluid, nasal discharge, tears, buffy coat, or a mucous membrane sample.

63. The kit of claim 46, wherein the labeled detector DNA comprises a fluorescence-emitting dye pair.

64. The kit of claim 63, wherein the fluorescence-emitting dye pair is a FRET pair.

65. The kit of claim 63, wherein the fluorescence-emitting dye pair is a quencher/fluorophore pair.

66. The kit of claim 46, wherein the labeled detector DNA comprises two or more fluorescence-emitting dye pairs.

67. The kit of claim 66, wherein the two or more fluorescence-emitting dye pairs include a first fluorescence-emitting dye pair that produces a first detectable signal and a second fluorescence-emitting dye pair that produces a second detectable signal.

68. The kit of claim 46, wherein the labeled detector DNA comprises a modified nucleobase, a modified sugar moiety, and/or a modified nucleic acid linkage.

69. The kit of claim 46, wherein the labeled detector DNA does not hybridize with the guide sequence of the guide RNA.

70. The kit of claim 46, wherein the guide RNA comprises a constant region comprising the nucleotide sequence set forth in SEQ ID NO:11.

71. A kit for detecting two or more target nucleic acids in a sample, comprising:
(i) a type V CRISPR/Cas effector protein, and/or a nucleic acid encoding the type V CRISPR/Cas effector protein;
(ii) a first guide RNA, and/or a nucleic acid encoding the first guide RNA, wherein the first guide RNA comprises a region that binds to the type V CRISPR/Cas effector protein, and a guide sequence that hybridizes with a segment of a first target nucleic acid;
(iii) a second guide RNA, and/or a nucleic acid encoding the second guide RNA, wherein the second guide RNA comprises a region that binds to the type V CRISPR/Cas effector protein, and a guide sequence that hybridizes with a segment of a second target nucleic acid; and
(iv) a labeled detector DNA that is single stranded.
